(19) United States
(12) Patent Application Publication
(10) Pub. No.: US 2024/0199663 A1
(43) Pub. Date: Jun. 20, 2024

(54) Cyclopolyphosphazenes, Related Methods of Preparation and Methods of Use

(71) Applicants: University of Saskatchewan, Saskatoon (CA); Battelle Energy Alliance, LLC, Idaho Falls, ID (US)

(72) Inventors: Samuel Attah-Poku, Saskatoon (CA); George Mutwiri, Saskatoon (CA); Sylvia Van Den Hurk, Saskatoon (CA); Jan Van Den Hurk, Saskatoon (CA); John Klaehn, Idaho Falls, ID (US); Volker Gerdts, Saskatoon (CA)

(21) Appl. No.: 18/556,667

(22) PCT Filed: Apr. 22, 2022
(86) PCT No.: PCT/CA2022/050618

§ 371 Date: Oct. 21, 2023

Related U.S. Application Data
(60) Provisional application No. 63/178,214, filed on Apr. 22, 2021.

(51) Int. Cl. C07F 9/6593; A61K 39/00; A61K 39/39

(52) U.S. Cl. CPC C07F 9/65815; A61K 39/39; A61K 2039/55511

(57) ABSTRACT

Disclosed herein are cyclopolyphosphazenes of formula I:

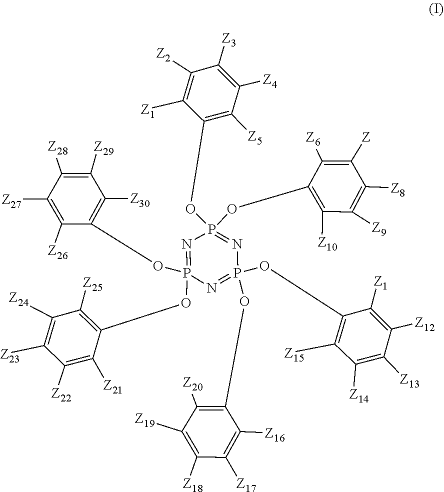

methods for the preparation thereof and uses thereof in adjuvant compositions.

### FIELD OF INVENTION

The present invention relates to cyclopolyphosphazenes, and related methods of preparation and use. More specifically, the present invention relates to cyclopolyphosphazenes for adjuvant compositions.

### BACKGROUND OF THE INVENTION

Certain types of vaccines, including killed or subunit vaccines, are often poorly immunogenic, and can result in weak and transient immune responses, thus requiring adjuvants to boost the immune response. Such immune responses include both T and B cell responses. Adjuvants are therefore valuable components of many vaccines. They may be used to improve the immunogenicity of vaccines with the aim of providing longer lasting immunity and long-term protection, enhancing the magnitude of the immune response and directing the immune response to a certain type (T helper response; In). Other advantages include the ability to utilize mucosal surfaces for vaccine delivery or to reduce the amount of antigen needed (antigen sparing). Altogether, such actions may increase the efficacy of both human and animal vaccines.

However, many currently available vaccines include adjuvants that are suboptimal with respect to the quality and magnitude of immune responses they induce. For example, alum, one of the few approved adjuvants for use in humans in the United States, induces good Th2 type immune responses but is not a potent adjuvant for Th1-type immune responses (HogenEsch et al., Vaccine (2002) 20 Suppl 3:S34-39).

Recently, a combination adjuvant platform has been developed that includes three components: (1) an immunostimulatory molecule, such as a CpG or poly(I:C) (polyinosinic-polycytidylic acid); (2) a polyphosphazene such as poly[di(sodium carboxylatophenoxy)phosphazene] (PCPP) or a poly(di-4-oxyphenylproprionate)phosphazene (PCEP) (as a sodium salt or in the acidic form); and (3) antimicrobial molecules capable of killing a broad spectrum of microbes known as “host defense peptides.” See, e.g., U.S. Pat. Nos. 9,408,908 and 9,061,001, incorporated herein by reference in their entireties. This triple adjuvant forms a stable complex and has been demonstrated to be highly effective in a wide range of human and animal vaccines following intramuscular or subcutaneous administration. See. e.g., Garg et al., J. Gen. Virol. (2014) 95:301-306. This triple adjuvant composition, when used with various vaccine antigens, induces effective long-term humoral and cellular immunity. Moreover, the adjuvant platform is suitable for maternal immunization and is highly effective in neonates even in the presence of maternal antibodies.

Polyphosphazenes such as PCEP and PCPP, have been shown to have adjuvant properties. However, some polyphosphazene polymers may have significant drawbacks. For example, there is batch-to-batch variability as a consequence of the difficulty in controlling the degree of polymerization. Batch variability may affect solubility of the polymer, molecular weight and stability. In addition, the synthesis cost of these large polymers may be high. Lastly, linear polymers may display different interactions with antigens.

There is a need in the art for additional effective and safe adjuvants that are easier and more cost effective to synthesize.

### SUMMARY OF THE INVENTION

The present invention relates to cyclopolyphosphazenes and related compositions and methods of preparation and use thereof. An adjuvant composition comprising a host defense peptide, an immunostimulatory sequence and cyclopolyphosphazenes are disclosed herein. Methods of enhancing an immune response to a selected antigen are disclosed herein.

This summary of the invention does not necessarily describe all features of the invention and is not intended to be limiting.

In certain embodiments of the invention, there is provided compounds of formula I:

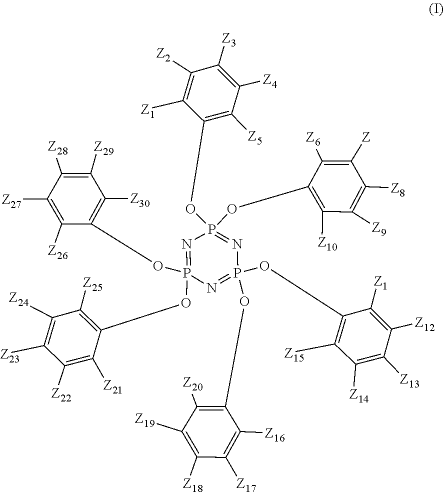

a tautomer, stereoisomer, polymorph, hydrate, solvate, or pharmaceutically acceptable salt thereof, wherein:
   each of Z₁, Z₂, Z₃, Z₄, Z₅, Z₆, Z₇, Z₈, Z₉, Z₁₀, Z₁₁, Z₁₂, Z₁₃, Z₁₄, Z₁₅, Z₁₆, Z₁₇, Z₁₈, Z₁₉, Z₂₀, Z₂₁, Z₂₂, Z₂₃, Z₂₄, Z₂₅, Z₂₆, Z₂₇, Z₂₈, Z₂₉, and Z₃₀ are independently selected from H or formula (II):

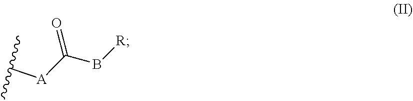

wherein at least one of Z₁₋₃₀ is represented by formula II, and each of Z₁₋₃₀ is identical or non-identical; and wherein:
   A, if present, is selected from C₁-C₇ alkyl, C₂-C₇ alkenyl, C₂-C₇ alkynyl, O, S, and N,
      wherein C₁-C₇ alkyl, C₂-C₇ alkenyl, and/or C₂-C₇ alkynyl are straight or branched and optionally substituted by one or more substituents selected from:
      1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc),
   B is selected from C₁-C₇ alkyl, C₂-C₇ alkenyl, C₂-C₇ alkynyl, H, O, S, and N,
      wherein C₁-C₇ alkyl, C₂-C₇ alkenyl, and/or C₂-C₇ alkynyl are straight or branched and optionally substituted by one or more substituents selected from:
      1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc),
   R, if present, is selected from H, C₁-C₄₅ alkyl, C₂-C₄₅ alkenyl, and C₂-C₄₅ alkynyl,
      wherein C₁-C₄₅ alkyl, C₂-C₄₅ alkenyl, and/or C₂-C₄₅ alkynyl are straight or branched and optionally substituted by one or more substituents selected from:
      1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc).

In certain embodiments, A may be C₂ alkyl; and B may be selected from O and N. In some embodiments, R may be selected from:
C₁-C₄₅ alkyl, C₁-C₄₅ alkenyl, and C₁-C₄₅ alkynyl,
   wherein C₁-C₄₅ alkyl, C₂-C₄₅ alkenyl, and/or C₂-C₄₅ alkynyl are straight or branched and optionally substituted by one or more substituents selected from hydroxyl, 1° amino, 20 amino, 30 amino, 40 amino, carboxylic acid, amino fluorenylmethyloxycarbonyl (NH-Fmoc), tert-butyloxycarbonyl (Boc), and amino tert-butyloxycarbonyl (—NH—Boc).

In certain embodiments wherein R may be selected from:

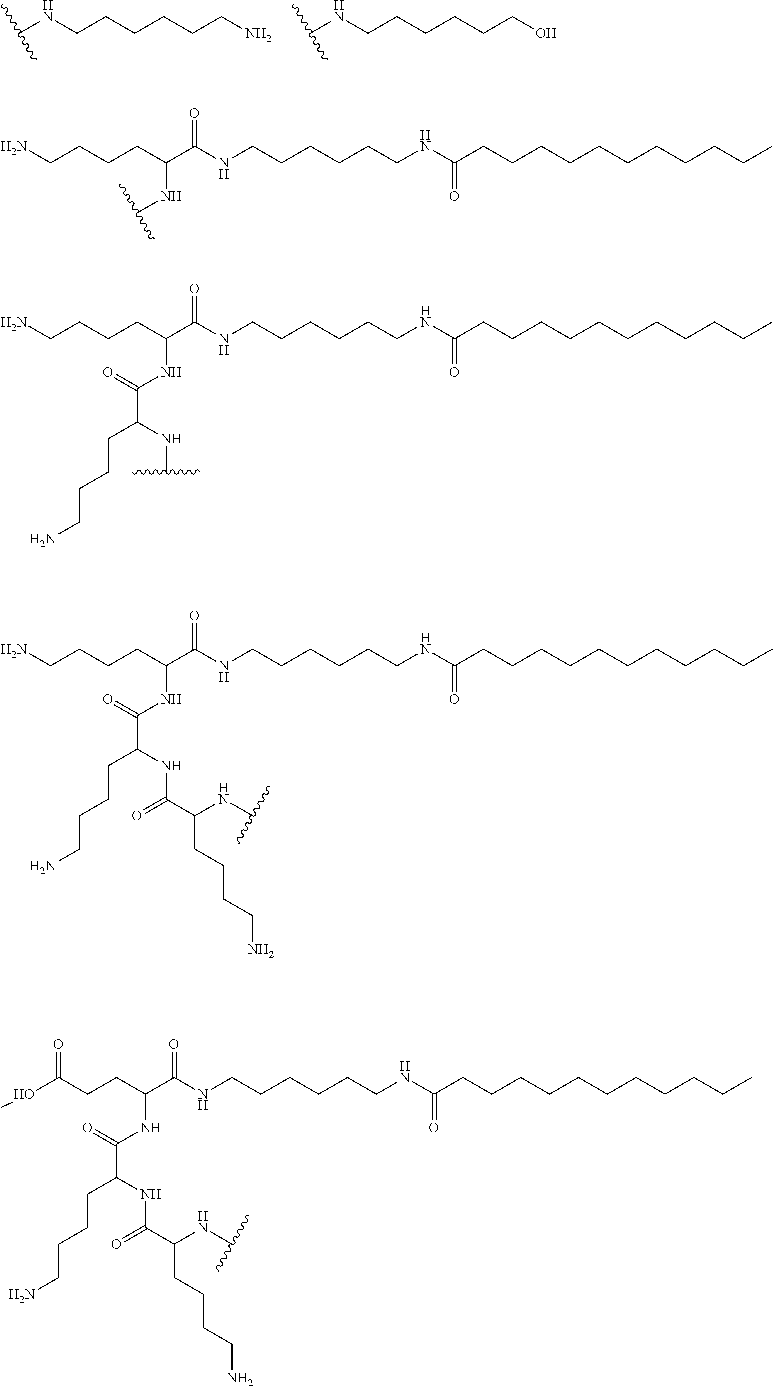

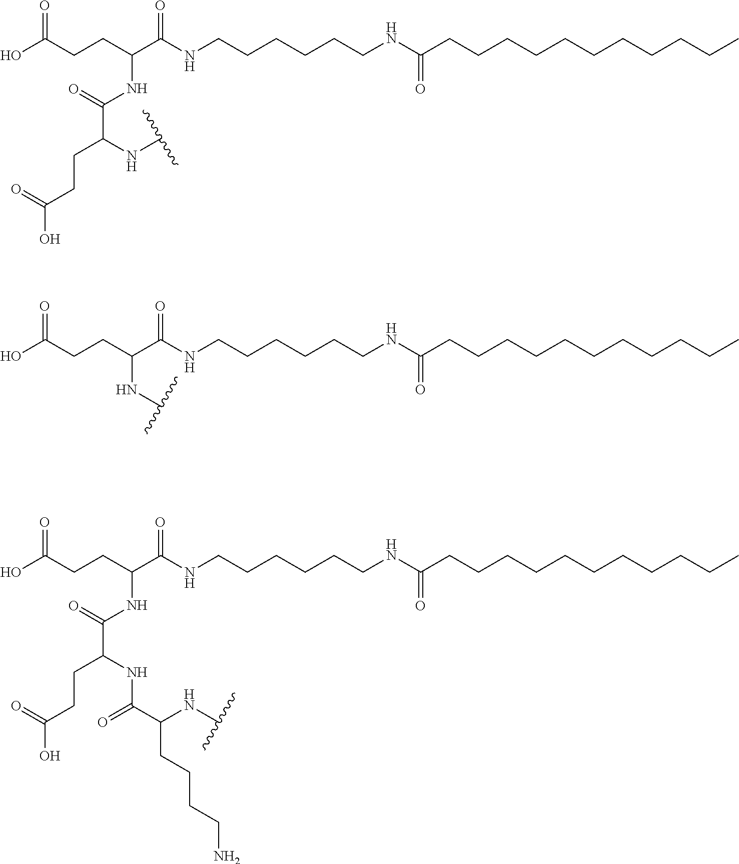 [SMILES: *NC(CCC(=O)O)C(=O)NC(CCC(=O)O)C(=O)NCCCCCCNC(=O)CCCCCCCCCCC ; *NC(CCC(=O)O)C(=O)NCCCCCCNC(=O)CCCCCCCCCCC ; *NC(CCCCN)C(=O)NC(CCC(=O)O)C(=O)NC(CCC(=O)O)C(=O)NCCCCCCNC(=O)CCCCCCCCCCC]

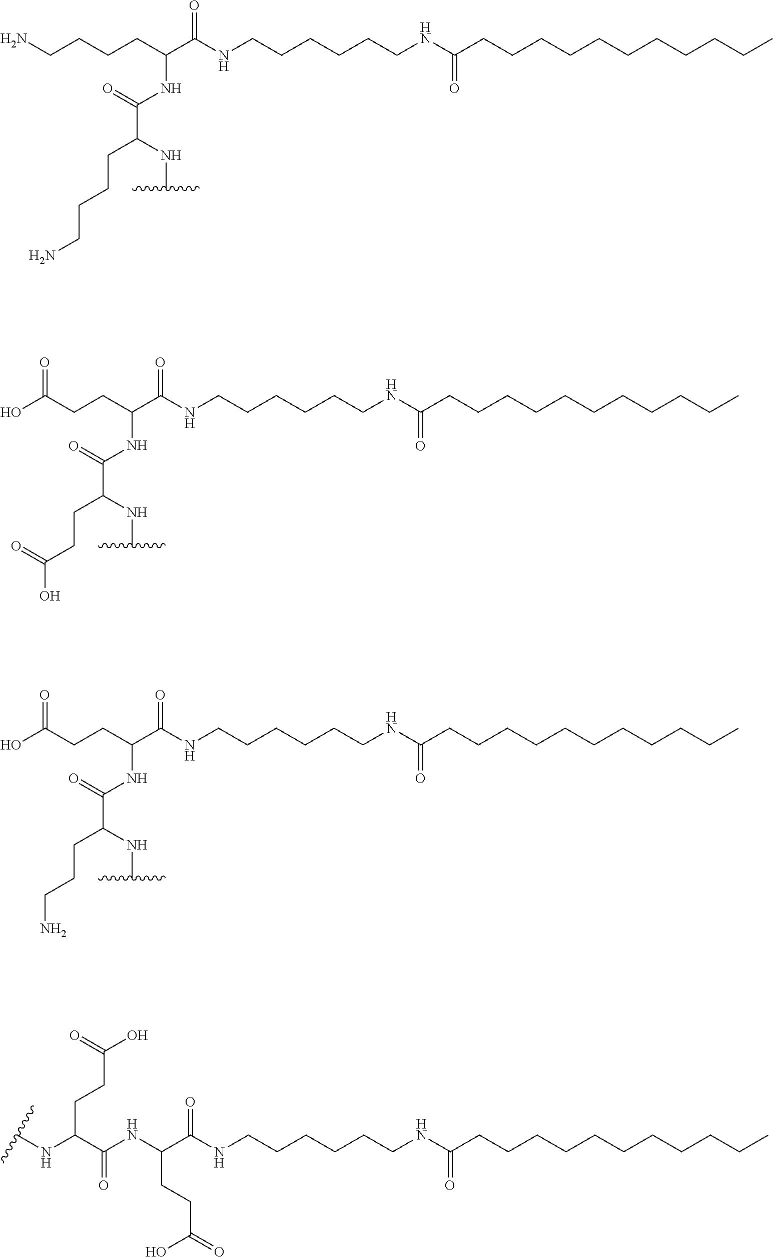 [SMILES: *NC(CCCCN)C(=O)NC(CCCCN)C(=O)NCCCCCCNC(=O)CCCCCCCCCCC ; *NC(CCC(=O)O)C(=O)NC(CCC(=O)O)C(=O)NCCCCCCNC(=O)CCCCCCCCCCC ; *NC(CCCN)C(=O)NC(CCC(=O)O)C(=O)NCCCCCCNC(=O)CCCCCCCCCCC ; *NC(CCC(=O)O)C(=O)NC(CCC(=O)O)C(=O)NCCCCCCNC(=O)CCCCCCCCCCC]

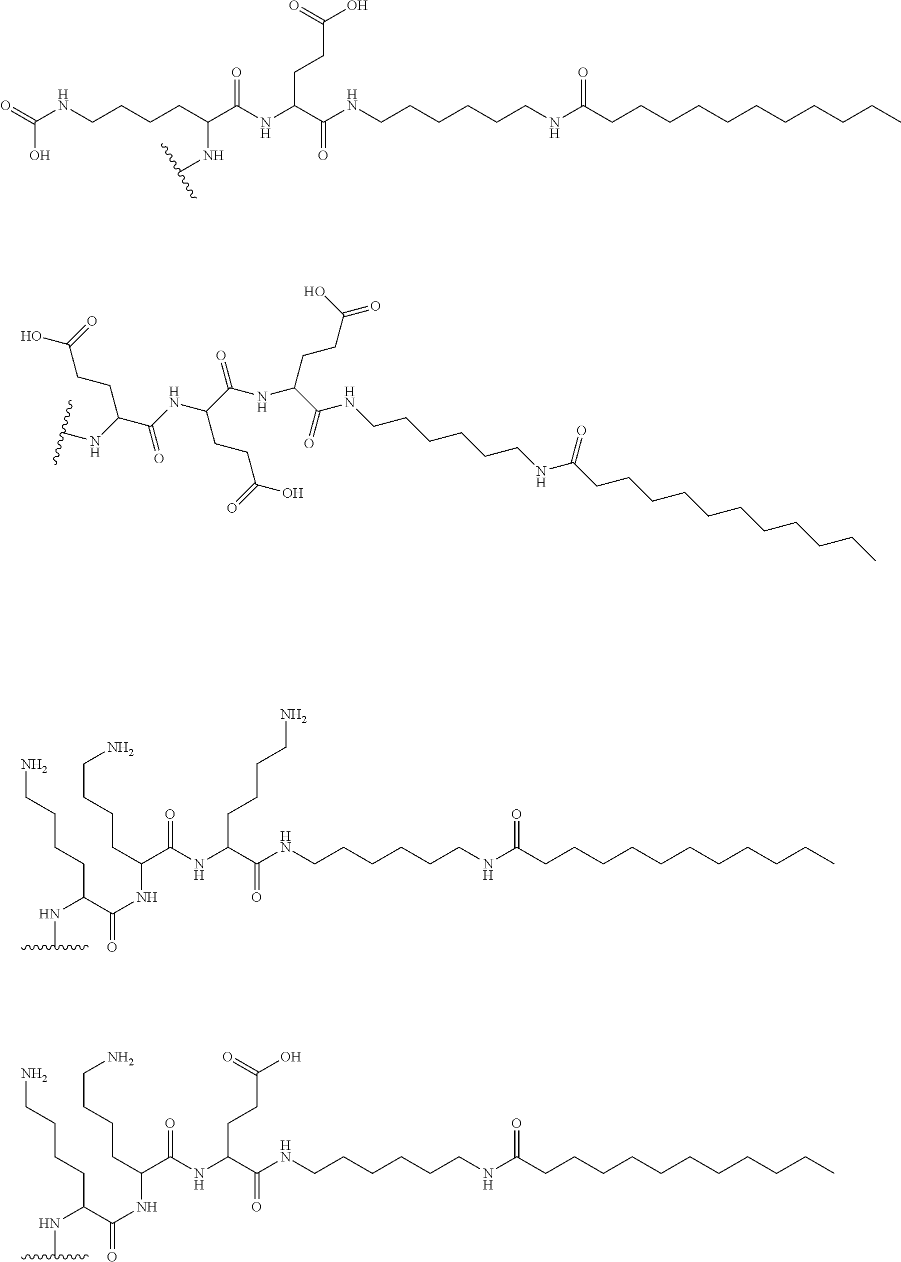 [SMILES: *NC(CCCCNC(=O)O)C(=O)NC(CCC(=O)O)C(=O)NCCCCCCNC(=O)CCCCCCCCCCC ; *NC(CCC(=O)O)C(=O)NC(CCC(=O)O)C(=O)NC(CCC(=O)O)C(=O)NCCCCCCNC(=O)CCCCCCCCCCC ; *NC(CCCCN)C(=O)NC(CCCCN)C(=O)NC(CCCCN)C(=O)NCCCCCCNC(=O)CCCCCCCCCCC ; *NC(CCCCN)C(=O)NC(CCCCN)C(=O)NC(CCC(=O)O)C(=O)NCCCCCCNC(=O)CCCCCCCCCCC]

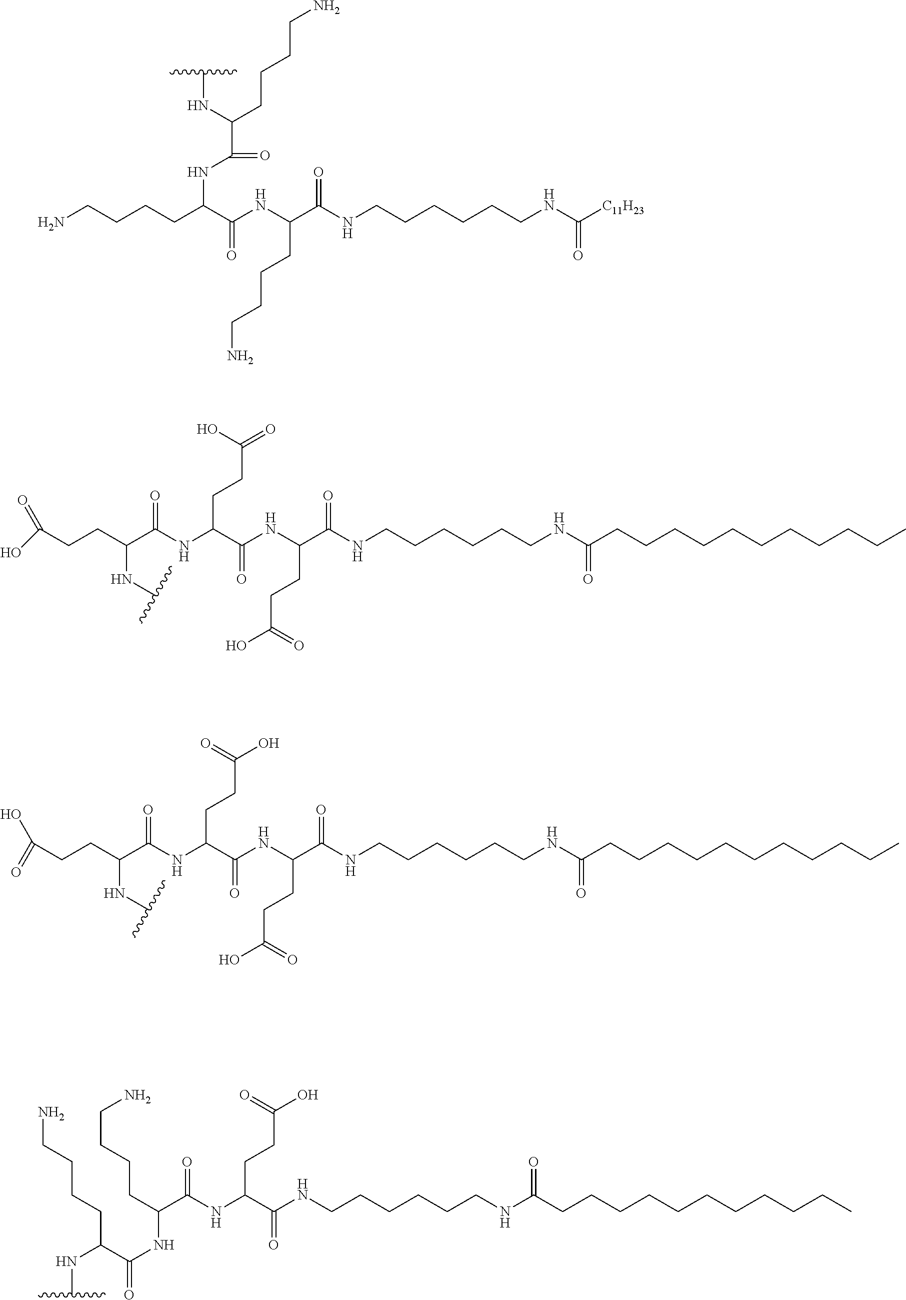

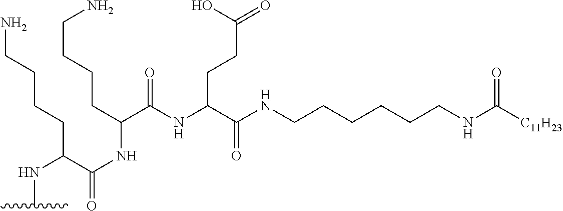

In some embodiments, A may be C₂ alkyl; B may be N; and R may be:

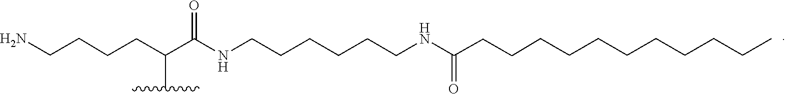 [SMILES: *C(CCCCN)C(=O)NCCCCCCNC(=O)CCCCCCCCCCC]

Some embodiments of the present invention disclosed herein comprise a compound having formula 37:

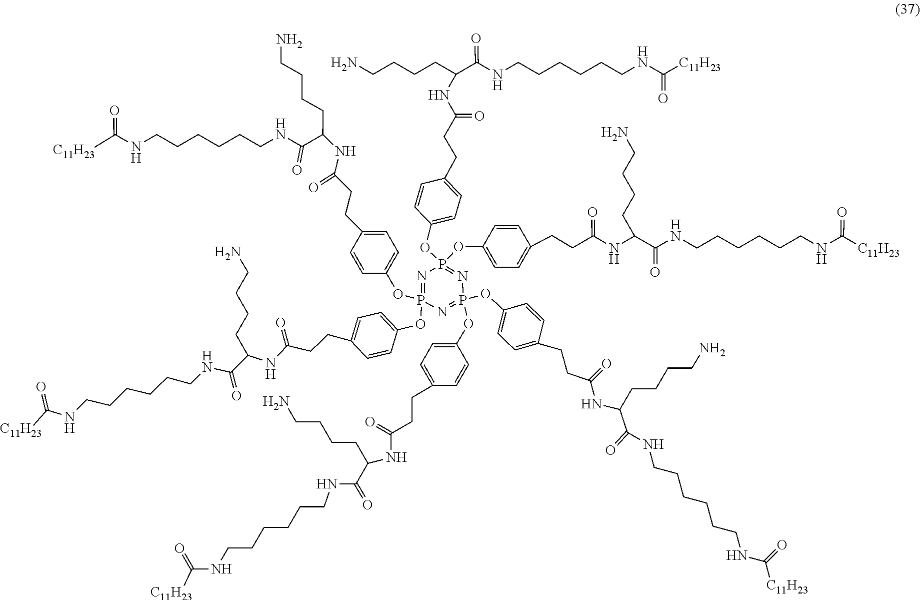

Further embodiments may comprise a tautomer, stereoisomer, polymorph, hydrate, solvate, or pharmaceutically acceptable salt of formula 37.

Some embodiments of the present invention disclosed herein comprise a compound having formula 6:

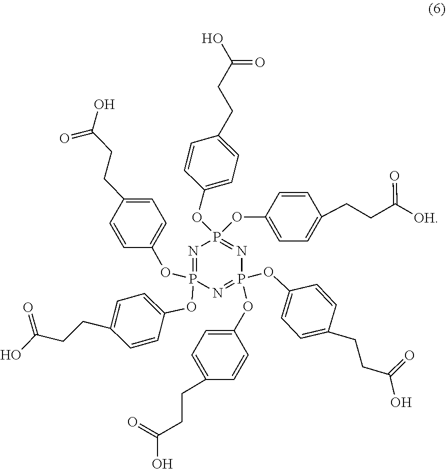 [SMILES: O=C(O)CCc1ccc(OP2(Oc3ccc(CCC(=O)O)cc3)=NP(Oc3ccc(CCC(=O)O)cc3)(Oc3ccc(CCC(=O)O)cc3)=NP(Oc3ccc(CCC(=O)O)cc3)(Oc3ccc(CCC(=O)O)cc3)=N2)cc1]

Some embodiments of the present invention comprise a tautomer, stereoisomer, polymorph, hydrate, solvate, or pharmaceutically acceptable salt of formula 6.

Some embodiments of the present invention disclosed herein comprise a compound of formula 11:

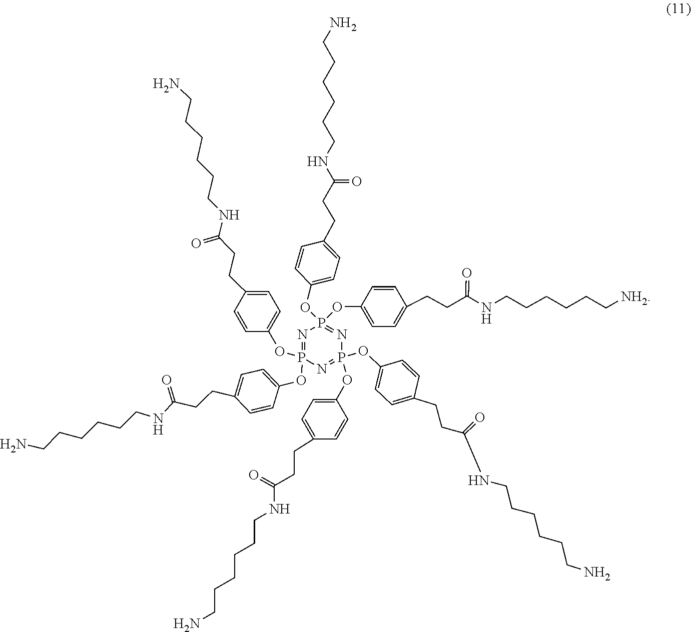 [SMILES: NCCCCCCNC(=O)CCc1ccc(OP2(Oc3ccc(CCC(=O)NCCCCCCN)cc3)=NP(Oc3ccc(CCC(=O)NCCCCCCN)cc3)(Oc3ccc(CCC(=O)NCCCCCCN)cc3)=NP(Oc3ccc(CCC(=O)NCCCCCCN)cc3)(Oc3ccc(CCC(=O)NCCCCCCN)cc3)=N2)cc1]

Some embodiments of the present invention comprise a tautomer, stereoisomer, polymorph, hydrate, solvate, or pharmaceutically acceptable salt of compound 11.

Some embodiments of the present invention comprise a compound of formula 9:

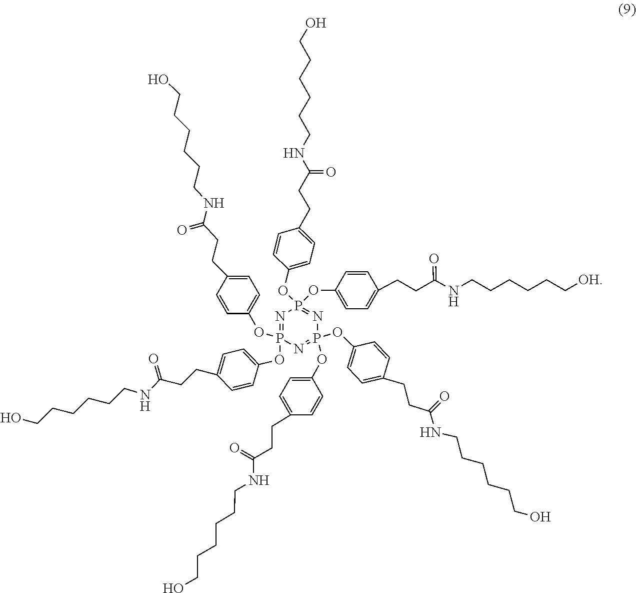 [SMILES: O=C(CCc1ccc(OP2(Oc3ccc(CCC(=O)NCCCCCCO)cc3)=NP(Oc3ccc(CCC(=O)NCCCCCCO)cc3)(Oc3ccc(CCC(=O)NCCCCCCO)cc3)=NP(Oc3ccc(CCC(=O)NCCCCCCO)cc3)(Oc3ccc(CCC(=O)NCCCCCCO)cc3)=N2)cc1)NCCCCCCO]

Some embodiments of the present invention comprise a tautomer, stereoisomer, polymorph, hydrate, solvate, or pharmaceutically acceptable salt of compound 9.

Some embodiments of the present invention comprise a compound of formula 39:

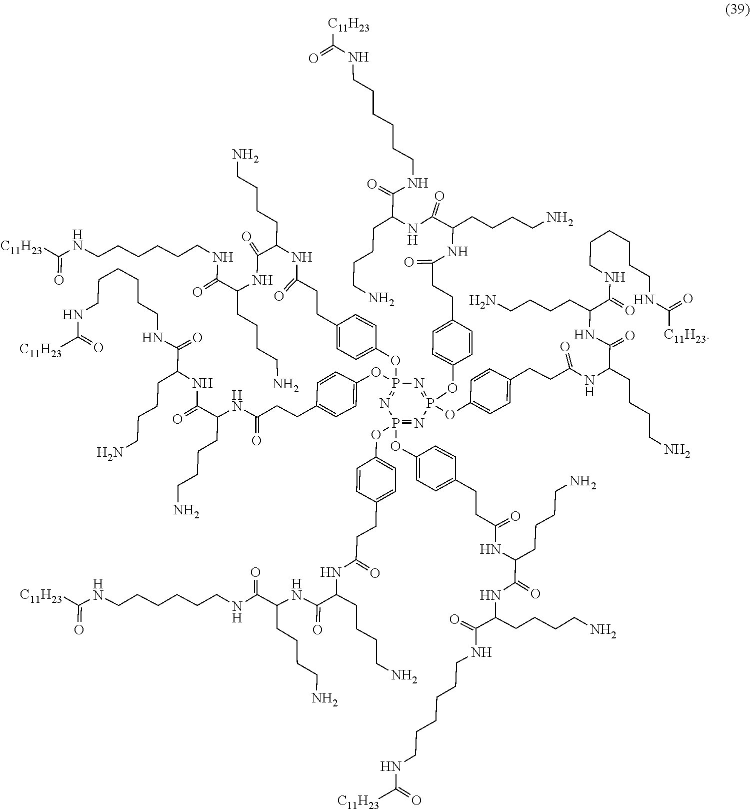

Some embodiments of the present invention comprise a tautomer, stereoisomer, polymorph, hydrate, solvate, or pharmaceutically acceptable salt of compound 39.

In further embodiments described herein are oligomeric structures comprising two or more compounds as defined hereinabove. In some embodiments, the two or more compounds may be linked via an amide or ester bond.

Described herein are methods for preparing a compound as defined hereinabove, said method comprising:
providing a reactant of formula VI:

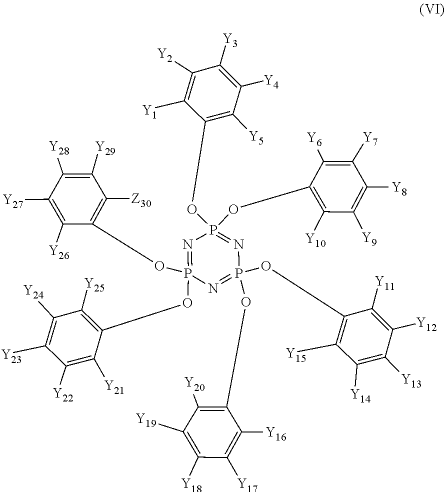

wherein each of Y₁₋₃₀ is independently selected from H and formula (III):

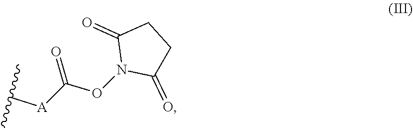

wherein at least one of Y₁₋₃₀ is represented by formula III and each of Y₁₋₃₀ is identical or non-identical;
performing a substitution reaction with a C₂-C₄₅ nucleophile; wherein
   A, if present, is selected from C₁-C₇ alkyl, C₂-C₇ alkenyl, C₂-C₇ alkynyl, O, S, and N,
      wherein C₁-C₇ alkyl, C₂-C₇ alkenyl, and/or C₂-C₇ alkynyl are straight or branched and optionally substituted by one or more substituents selected from:
         1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc),
   the C₂-C₄₅ nucleophile is linear or branched C₂-C₄₅ alkyl, C₂-C₄₅ alkenyl, and/or C₂-C₄₅ alkynyl and optionally substituted by one or more substituents selected from:
      1° amino, 2° amino, 3° amino, 4° amino, acetal, acyl halide, acyl, aldehyde, alkoxyl, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc).

In some embodiments of the methods, the reactant may comprise formula 7:

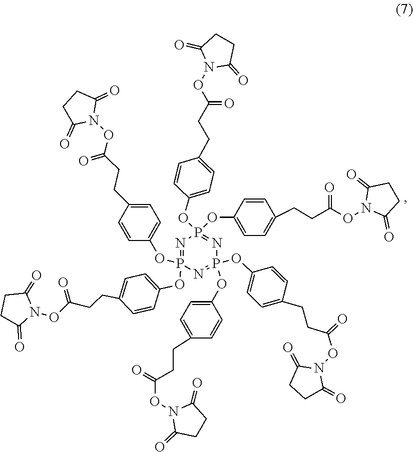 [SMILES: O=C(CCc1ccc(OP2(Oc3ccc(CCC(=O)ON4C(=O)CCC4=O)cc3)=NP(Oc3ccc(CCC(=O)ON4C(=O)CCC4=O)cc3)(Oc3ccc(CCC(=O)ON4C(=O)CCC4=O)cc3)=NP(Oc3ccc(CCC(=O)ON4C(=O)CCC4=O)cc3)(Oc3ccc(CCC(=O)ON4C(=O)CCC4=O)cc3)=N2)cc1)ON1C(=O)CCC1=O]

and the C₂-C₄₅ nucleophile may be a C₂-C₄₃ amine,
wherein the C₂-C₄₅ amine is linear or branched C₂-C₄₅ alkyl, C₂-C₄₅ alkenyl, and/or C₂-C₄₅ alkynyl and optionally substituted by one or more substituents selected from:
   1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc).

In some embodiments, the C₂-C₄₅ nucleophile may be one or more of:

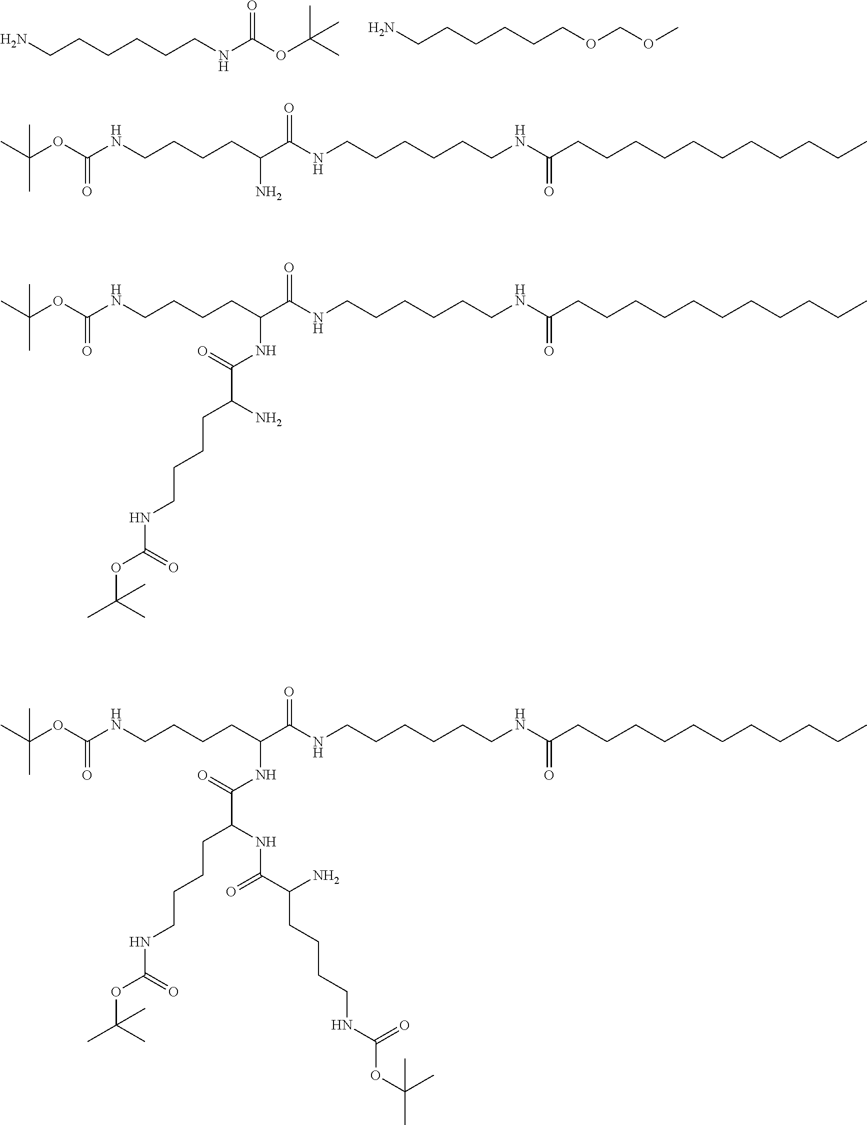

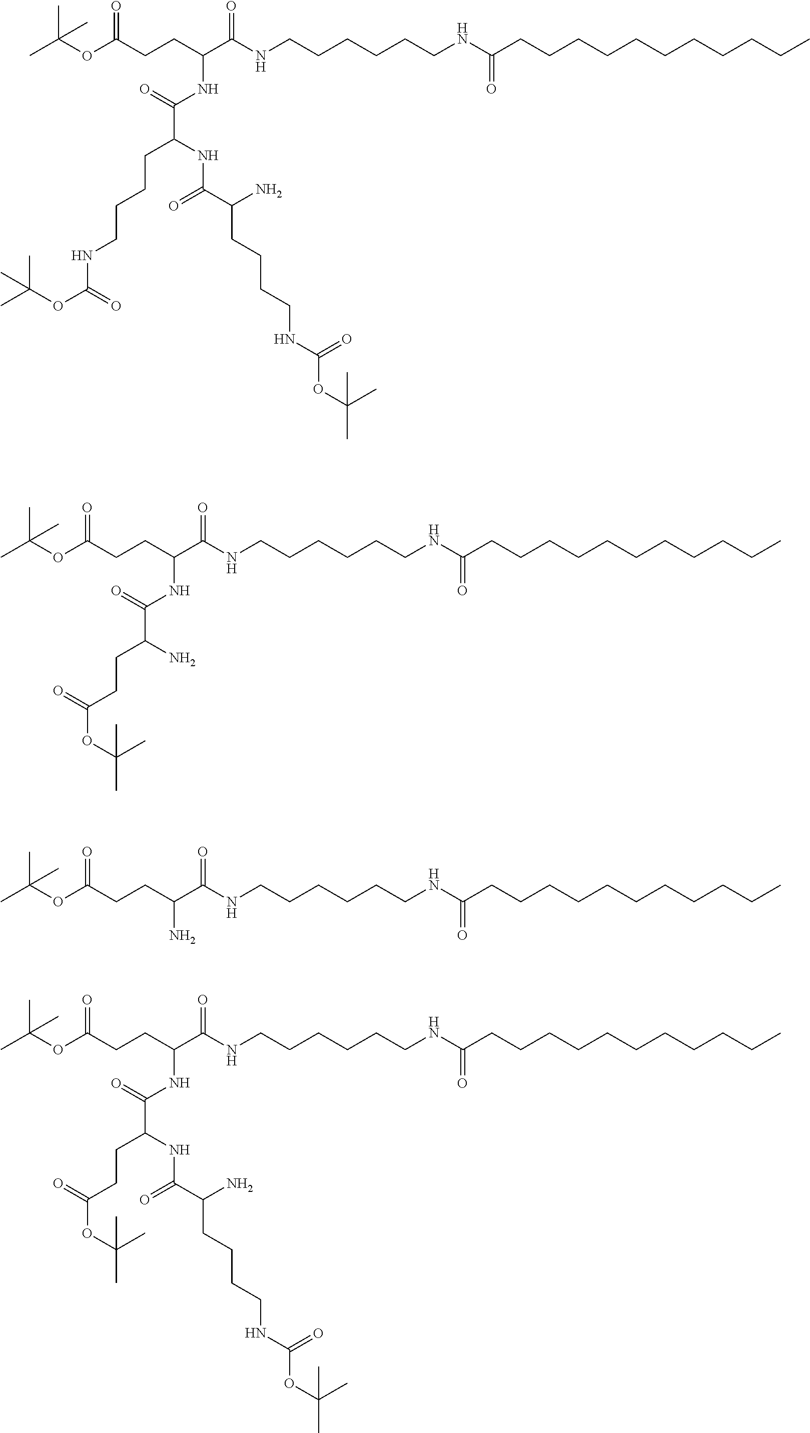

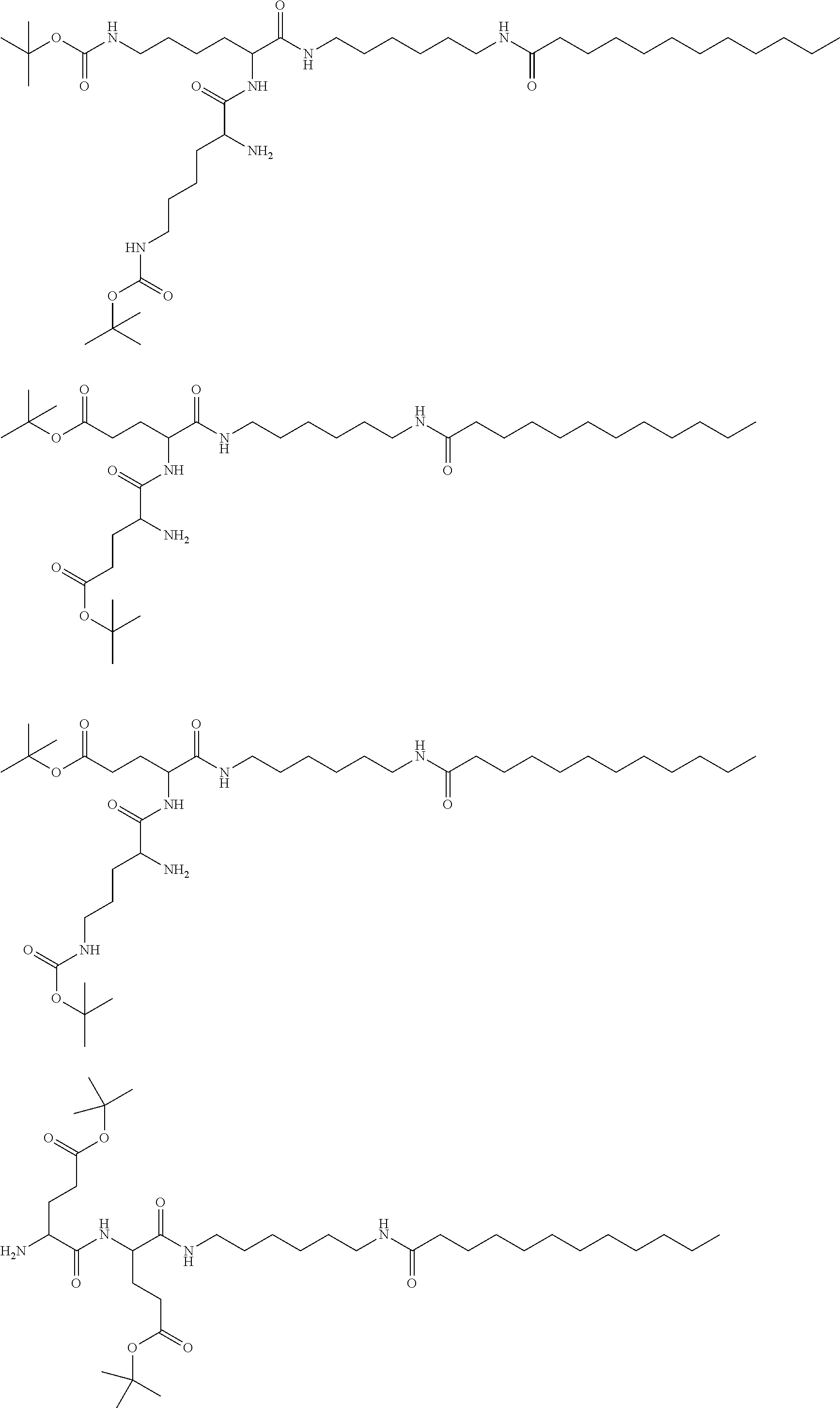

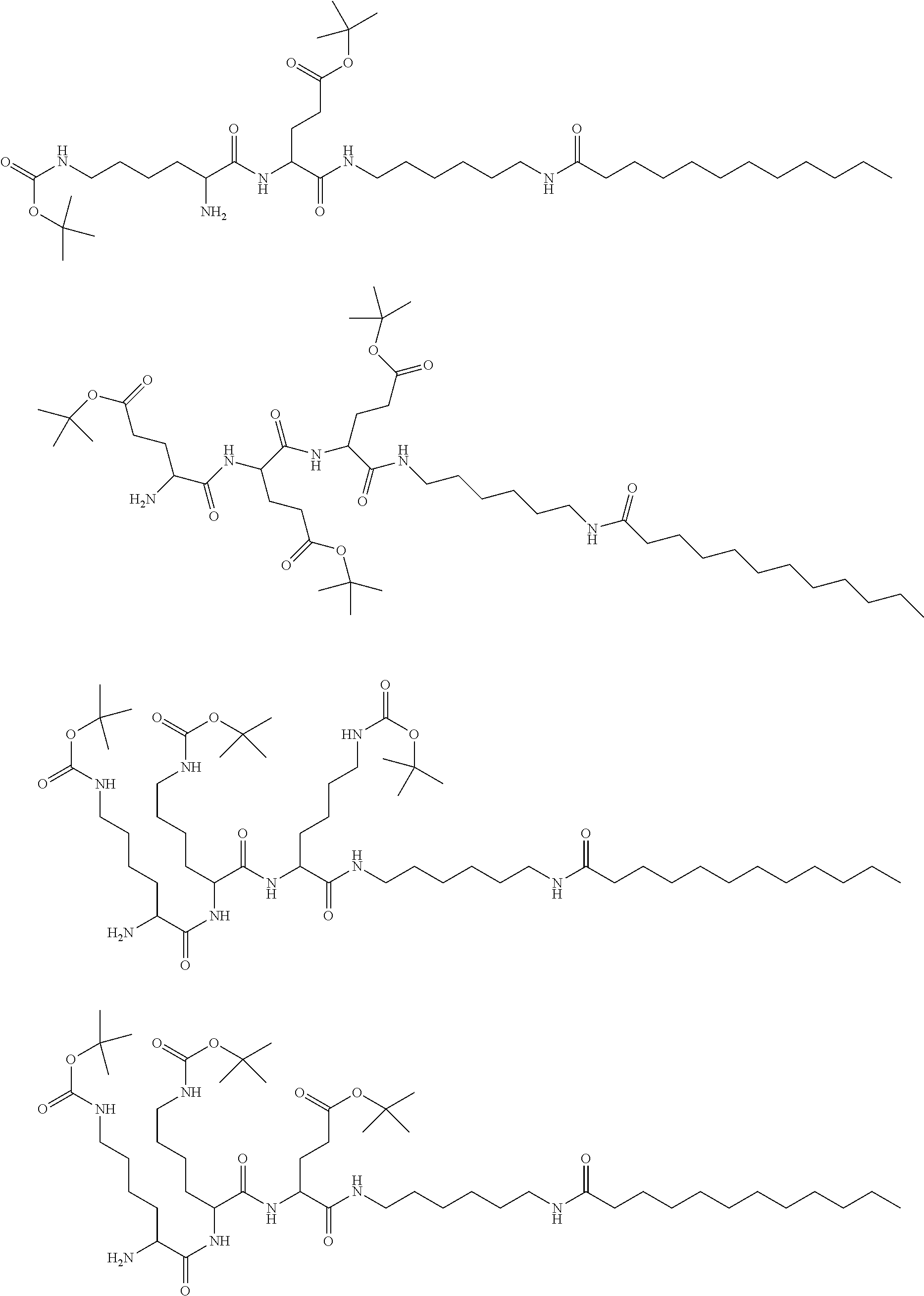

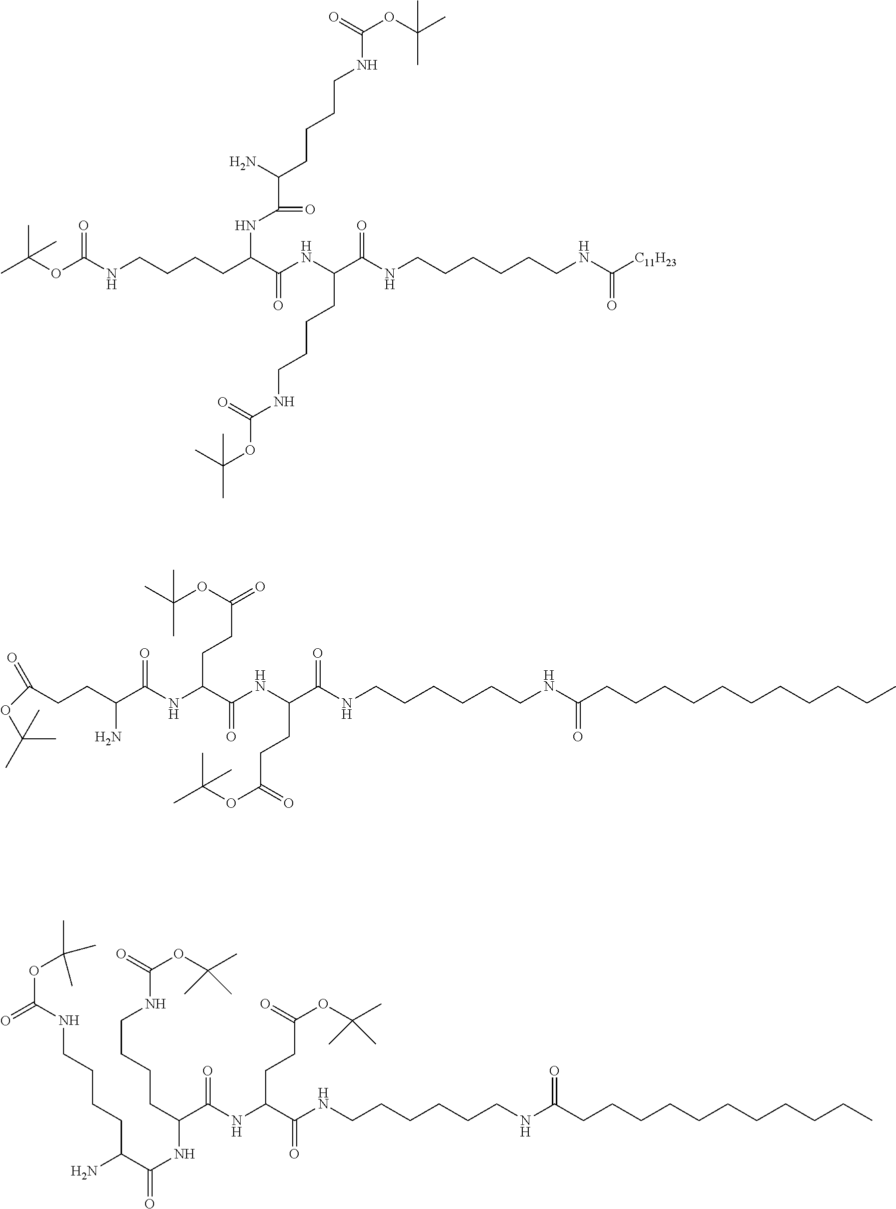

In some embodiments, the method may further comprise a deprotection step comprising an aqueous acid at a pH of 1 or less.

In further embodiments described herein, are methods of producing a compound as described above, the method comprising:
providing a first reactant of formula 3:

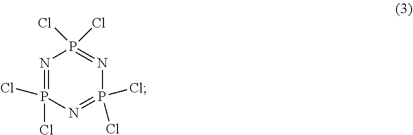 [SMILES: ClP1(Cl)=NP(Cl)(Cl)=NP(Cl)(Cl)=N1]

performing a first substitution reaction with formula IX:

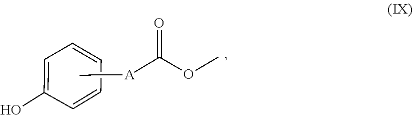

the first reactant, and a base to yield a first intermediate;
performing a hydrolysis reaction with a hydroxide salt and the first intermediate to yield a second intermediate;
performing a second substitution reaction with the second intermediate, N-hydroxysuccinimide and N,N′-Diisopropylcarbodiimide (DIPCDI) to yield a third intermediate; and
performing a third substitution reaction with a C₂-C₄₅ nucleophile and the third intermediate, wherein:
   A is selected from C₁-C₇ alkyl, C₂-C₇ alkenyl, C₂-C₇ alkynyl,
      wherein C₁-C₇ alkyl, C₂-C₇ alkenyl, and/or C₂-C₇ alkynyl are straight or branched and optionally substituted by one or more substituents selected from:
         1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc),
   the C₂-C₄₅ nucleophile is a linear or branched C₂-C₄₅ alkyl, C₂-C₄₅ alkenyl, and/or C₂-C₄₅ alkynyl and optionally substituted by one or more substituents selected from:
      1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc).

In some embodiments, the first substitution reaction may further comprise nBu₄, N⁺Br⁻ (TBAB) and the base is K₂CO₃. In further embodiments, the hydroxide salt is potassium hydroxide or sodium hydroxide. In some embodiments, the first intermediate may comprise formula X:

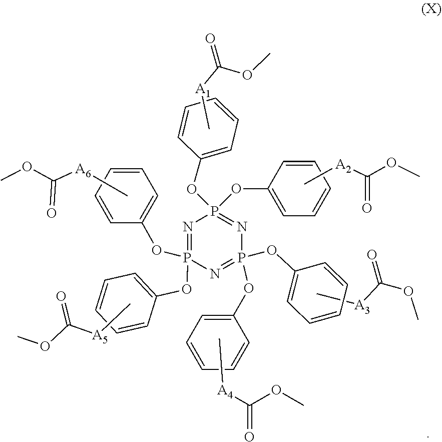

In some embodiments, the second intermediate may comprise formula XI:

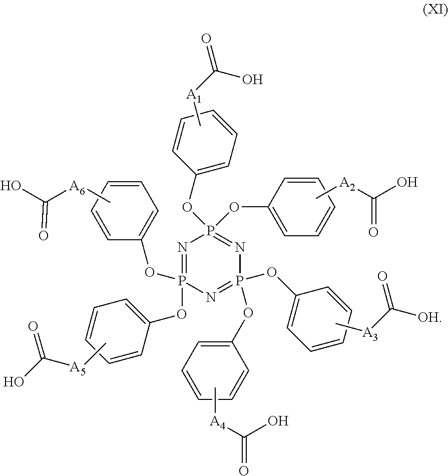

In some embodiments, the third intermediate may comprise formula VI:

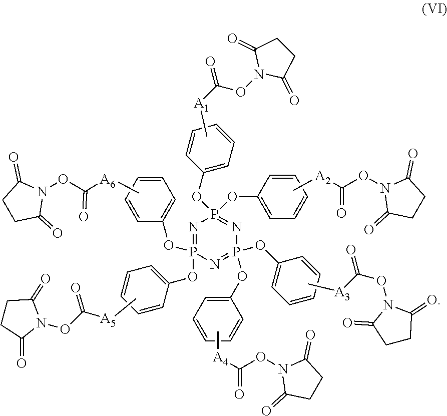

In further embodiments of the present invention, the C₂-C₄₅ nucleophile may be one or more of:

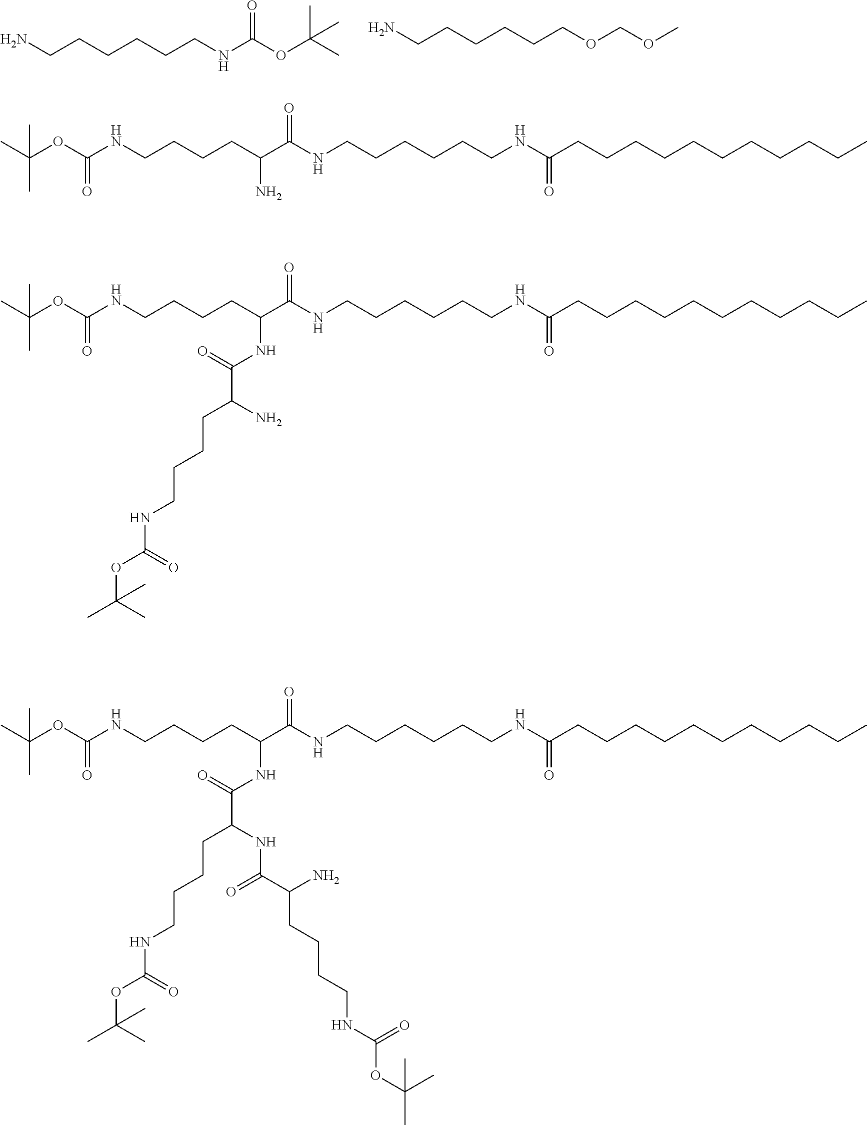

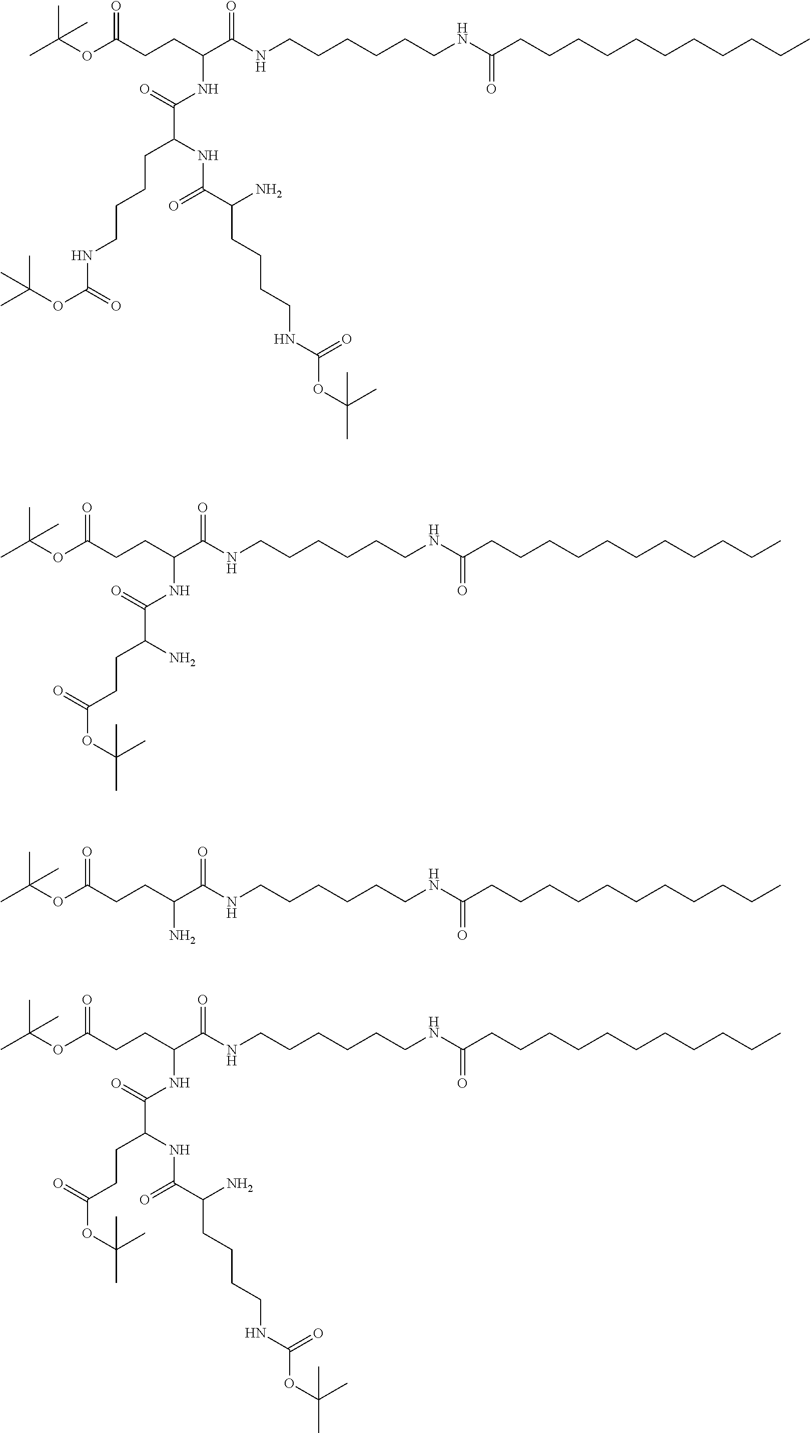

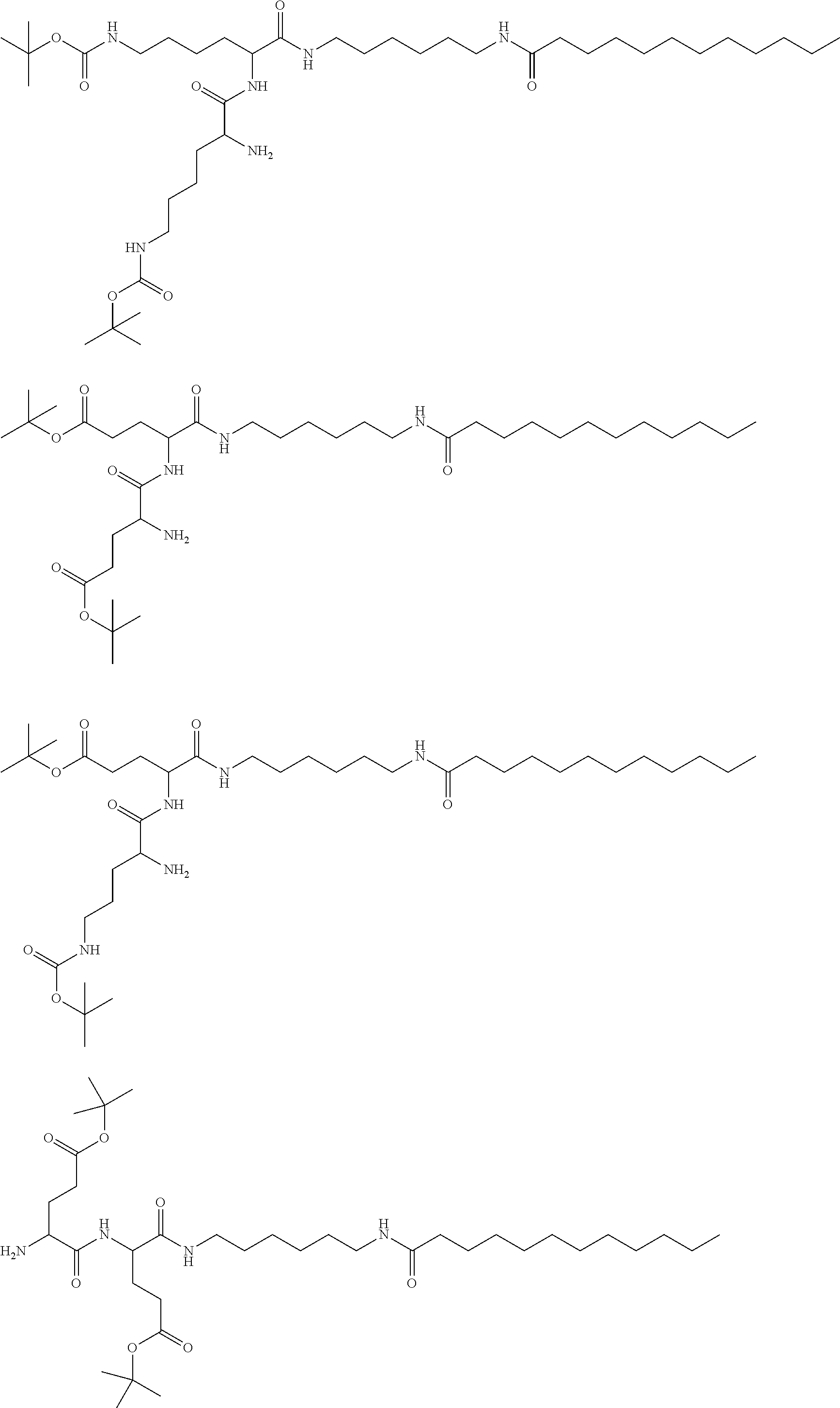

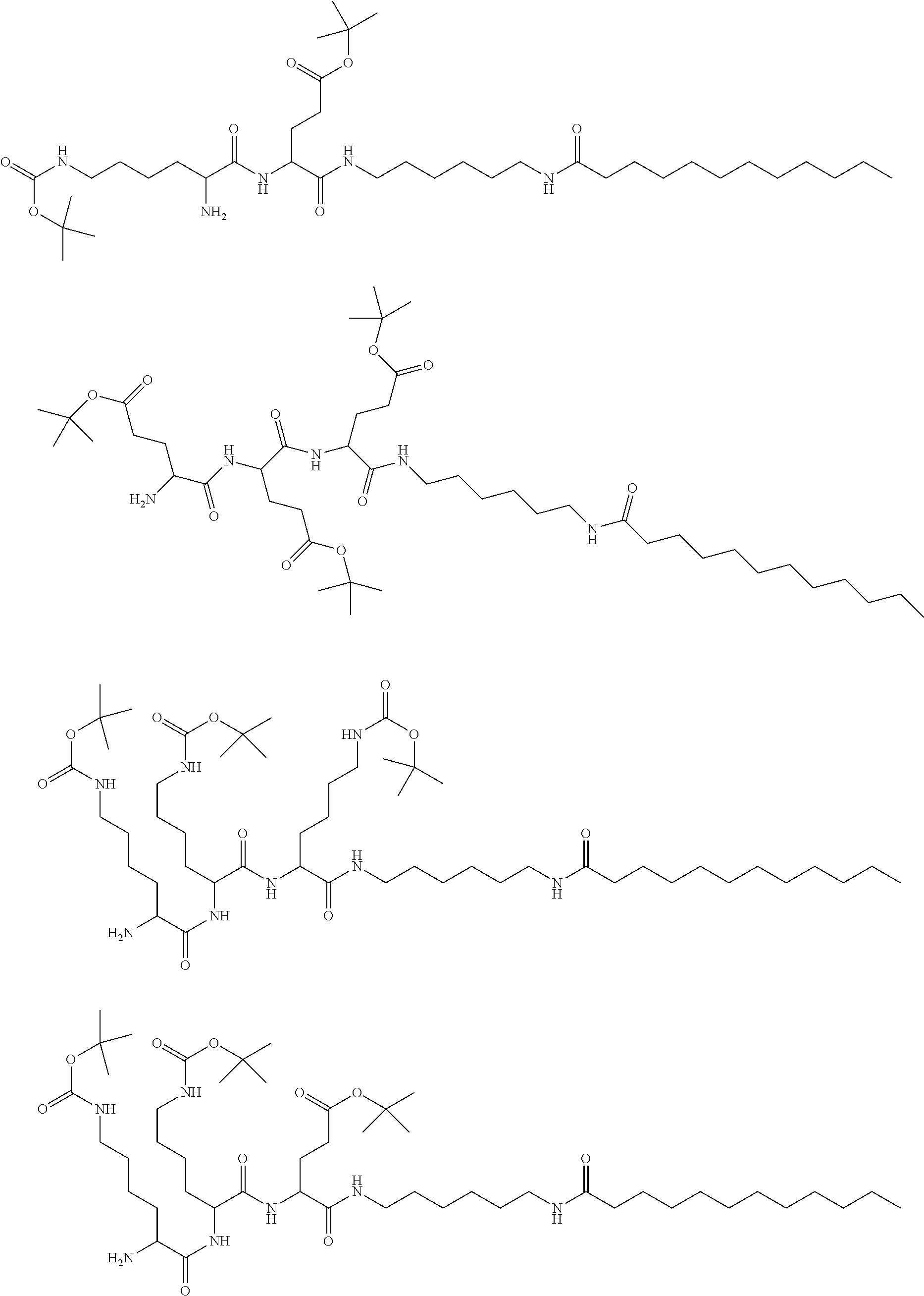

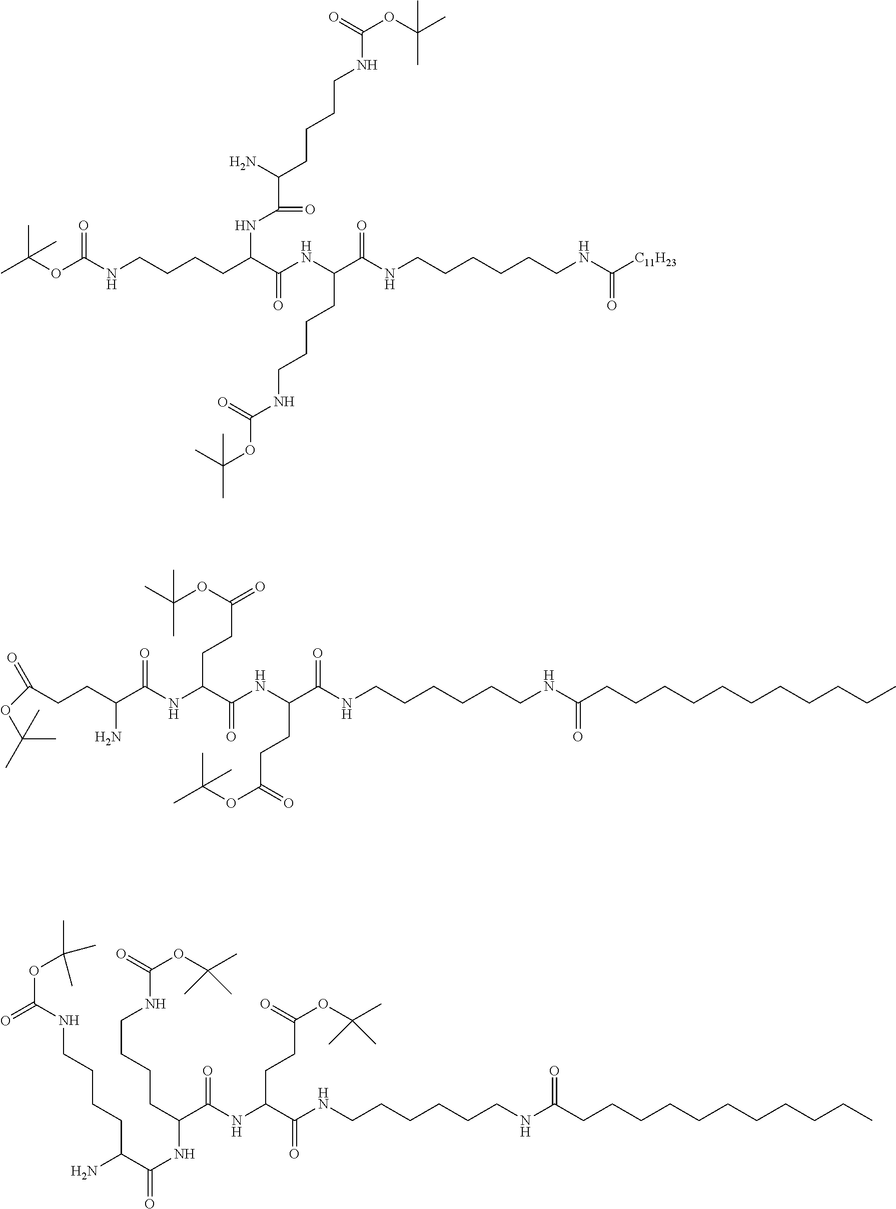

In further embodiments, the method may comprise acidic work-up at a pH of 1 or less.

Described herein are methods of producing a compound of formula X:

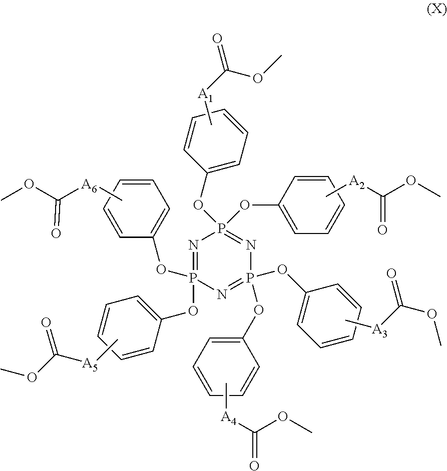

the method comprising:
   providing a reactant of formula VIII:

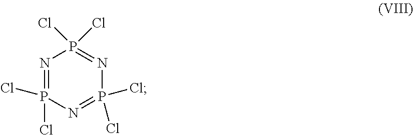 [SMILES: ClP1(Cl)=NP(Cl)(Cl)=NP(Cl)(Cl)=N1]

performing a substitution reaction with formula IX:

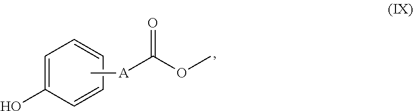

the reactant, and a base.

In some embodiments, the substitution reaction may further comprise nBu₄, N⁺Br⁻ (TBAB). In further embodiments, the base may be K₂CO₃.

Described herein are methods of producing a compound of formula XI:

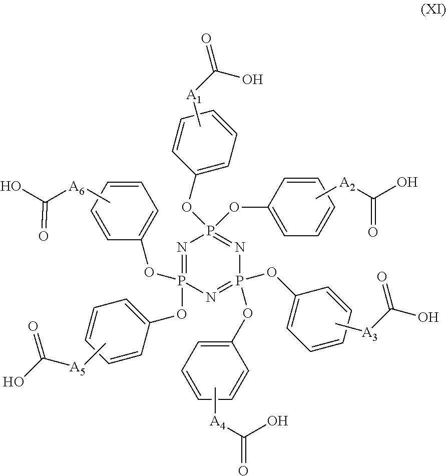

the method comprising:
   providing a reactant of formula X:

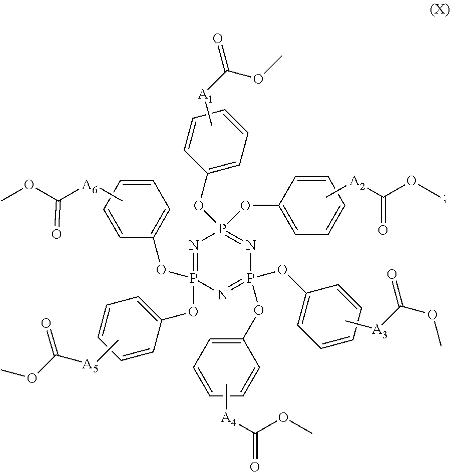

performing a hydrolysis reaction with a hydroxide salt and the reactant.

In some embodiments, the hydroxide salt is sodium hydroxide.

Described herein are methods of producing a compound of formula VI:

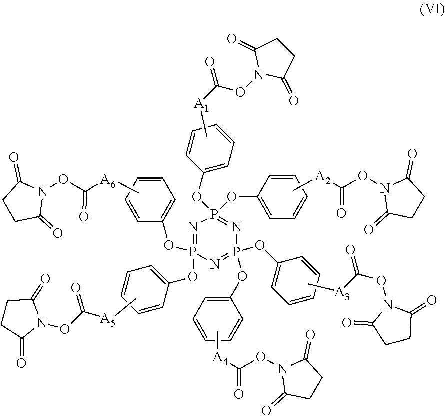

the method comprising:
providing a reactant of formula XI:

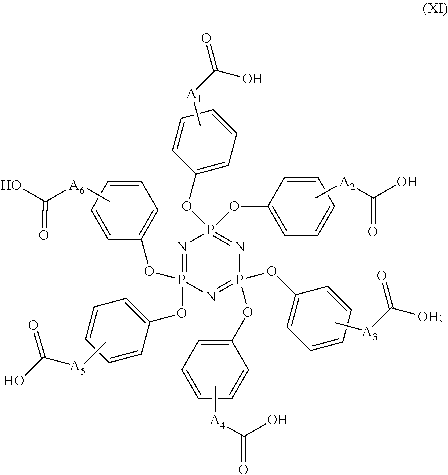

performing a substitution reaction with N-hydroxysuccinimide, N,N′-Diisopropylcarbodiimide (DIPCDI) and the reactant.

Described herein are methods of producing a compound of formula 5:

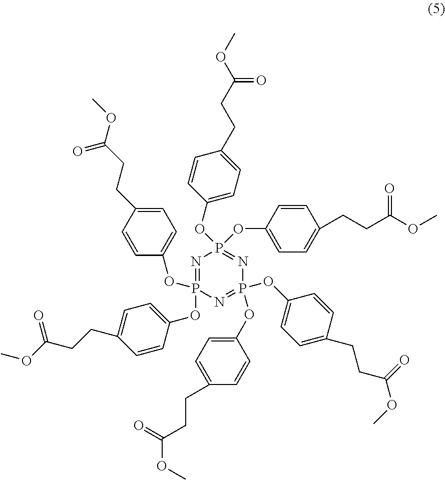

the method comprising:
   providing a reactant of formula 3:

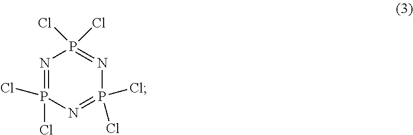 [SMILES: ClP1(Cl)=NP(Cl)(Cl)=NP(Cl)(Cl)=N1]

performing a substitution reaction with formula 4:

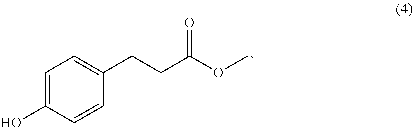

the reactant and a base.

In some embodiments, the substitution reaction may further comprise nBu₄, N⁺Br⁻ (TBAB). In further embodiments, the base may be K₂CO₃.

Described herein are methods of producing a compound of formula 6:

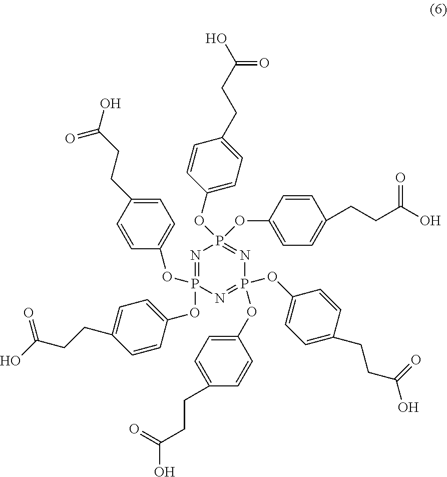 [SMILES: O=C(O)CCc1ccc(OP2(Oc3ccc(CCC(=O)O)cc3)=NP(Oc3ccc(CCC(=O)O)cc3)(Oc3ccc(CCC(=O)O)cc3)=NP(Oc3ccc(CCC(=O)O)cc3)(Oc3ccc(CCC(=O)O)cc3)=N2)cc1]

the method comprising:
   providing a reactant of formula 5:

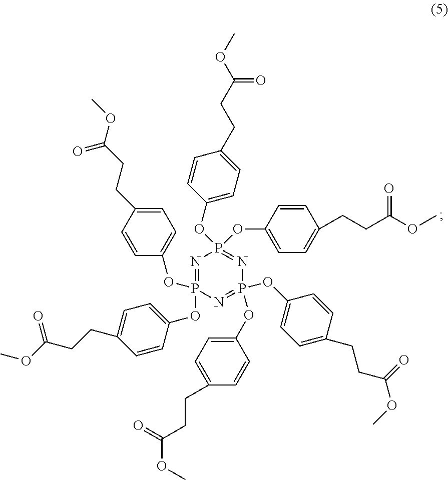

and
performing a hydrolysis reaction with an hydroxide salt and the reactant. In some embodiments, the hydroxide salt is sodium hydroxide.

Described herein are methods of producing a compound of formula 7:

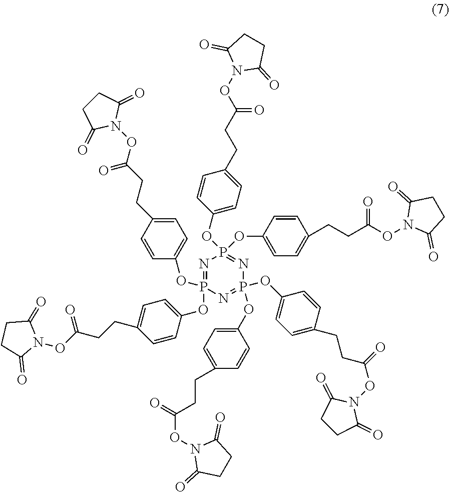 [SMILES: O=C(CCc1ccc(OP2(Oc3ccc(CCC(=O)ON4C(=O)CCC4=O)cc3)=NP(Oc3ccc(CCC(=O)ON4C(=O)CCC4=O)cc3)(Oc3ccc(CCC(=O)ON4C(=O)CCC4=O)cc3)=NP(Oc3ccc(CCC(=O)ON4C(=O)CCC4=O)cc3)(Oc3ccc(CCC(=O)ON4C(=O)CCC4=O)cc3)=N2)cc1)ON1C(=O)CCC1=O]

the method comprising:
   providing a reactant of formula 6:

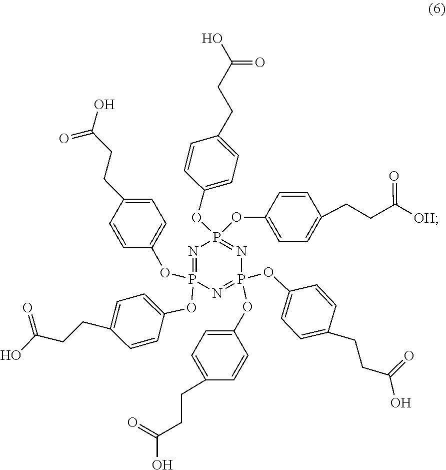 [SMILES: O=C(O)CCc1ccc(OP2(Oc3ccc(CCC(=O)O)cc3)=NP(Oc3ccc(CCC(=O)O)cc3)(Oc3ccc(CCC(=O)O)cc3)=NP(Oc3ccc(CCC(=O)O)cc3)(Oc3ccc(CCC(=O)O)cc3)=N2)cc1]

performing a substitution reaction with N-hydroxysuccinimide, N,N′-Diisopropylcarbodiimide (DIPCDI) and the reactant.

Described herein are methods of producing a compound of formula 37:

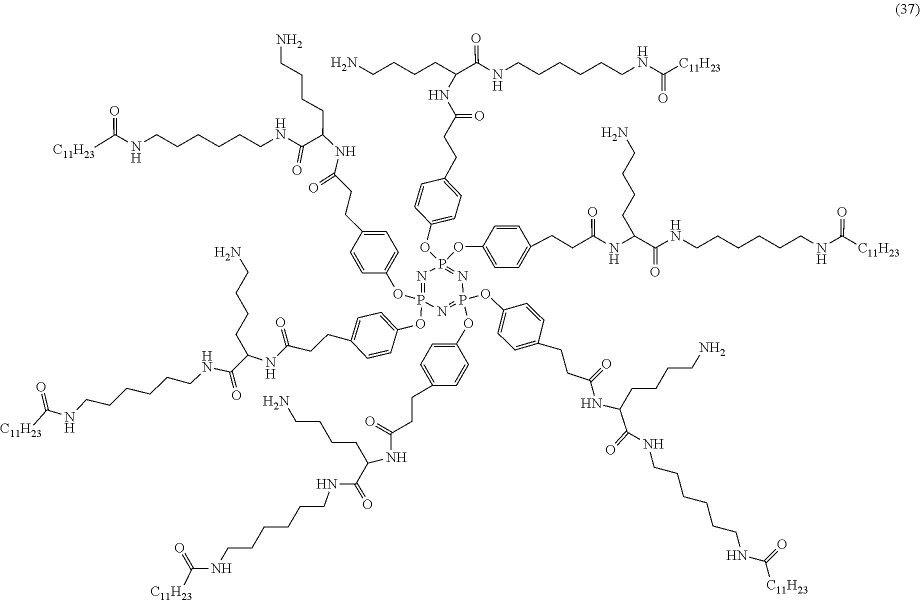

the method comprising:
   providing a first reactant of formula 7:

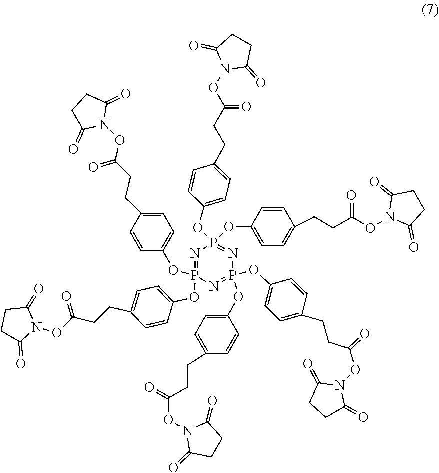 [SMILES: O=C(CCc1ccc(OP2(Oc3ccc(CCC(=O)ON4C(=O)CCC4=O)cc3)=NP(Oc3ccc(CCC(=O)ON4C(=O)CCC4=O)cc3)(Oc3ccc(CCC(=O)ON4C(=O)CCC4=O)cc3)=NP(Oc3ccc(CCC(=O)ON4C(=O)CCC4=O)cc3)(Oc3ccc(CCC(=O)ON4C(=O)CCC4=O)cc3)=N2)cc1)ON1C(=O)CCC1=O]

performing a substitution reaction with a second reactant of formula 19:

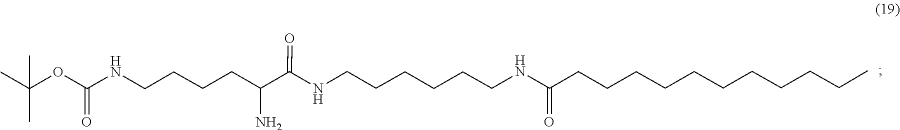 [SMILES: CCCCCCCCCCCC(=O)NCCCCCCNC(=O)C(N)CCCCNC(=O)OC(C)(C)C]

and
removing the t-butyl carbamate group with acid.

Described herein are uses of any of the compounds discussed hereinabove as an immunomodulatory.

Described herein are adjuvant compositions comprising a compound as discussed hereinabove, and one or more of a host defense peptide, an immunostimulatory sequence or a pharmaceutically acceptable excipient, diluent, or carrier. Some embodiments of the adjuvant composition may comprise a compound of formula 37. Some embodiments of the adjuvant composition may comprise a compound of formula 39. In some embodiments, the host defense peptide may be IDR-1002 (SEQ ID NO:19). In some embodiments, the immunostimulatory sequence is poly(I:C). In further embodiments, the adjuvant composition comprises an antigen. In some embodiments, the antigen is from a virus, bacteria, parasite, prion or fungus.

Described herein are compositions comprising the adjuvant composition as described herein and a pharmaceutically acceptable excipient. In some embodiments, the adjuvant composition comprises a host defense peptide and a compound as defined hereinabove. In some embodiments, the adjuvant composition comprises an immunostimulatory sequence and a compound as defined hereinabove.

Described herein are methods of enhancing an immune response to a selected antigen, said method comprising administering to a subject the composition of any one of the above embodiments. In certain non limiting embodiments the composition may be in admixture with the antigen to be administered. In some embodiments of the methods, administration comprises systemic and mucosal administration. In further embodiments, systemic administration comprises intramuscular administration. In further embodiments, systemic administration comprises oral administration. In some embodiments, mucosal administration comprises intranasal, respiratory, buccal and genital.

### BRIEF DESCRIPTION OF THE DRAWINGS

These and other features of the invention will become more apparent from the following description in which reference is made to the appended drawings wherein:

FIGS. 1A-B show the IgG titres as measured by ELISA in the serum of Leghorn chicken following intramuscular administration of 10 μg Salmonella typhimurium Cell invasion protein (SipD) as described in the examples, two weeks after the first immunization and before the second immunization (FIG. 1A) and Four weeks after the first administration (FIG. 1B). Data represent the median value and interquartile range (n=12). Horizontally striped bars: PBS vaccine (no antigen, no adjuvant); Dotted bars: antigen without adjuvant; Grey bars with black line: antigen mixed with 20 μg PCEP; Black bars with grey line: antigen mixed with 20 μg 11; White bars with grey line: antigen mixed with 20 μg 37; Grey bars with grey line: antigen mixed with PCEP-TriAdj; Black bars: antigen mixed with 11-TriAdj; White bars with black line: antigen mixed with 37-TriAdj. TriAdj is composed of 20 μg polyphosphazene: 40 μg HDP IDR1002: 20 μg Poly(I:C) weight ratio per dose.

FIG. 2 shows the IgG1 titres as measured by ELISA in the serum of Balb/c mice following intramuscular administration of 50 μg ovalbumin as described in the examples, four weeks after the first immunization and before the second immunization (4 weeks) and eight weeks after the first administration (8 weeks). Data represent the median value and interquartile range (n=6). Dotted bars: antigen only; Grey bars: antigen mixed with PCEP-TriAdj; White bars: antigen mixed with 37-TriAdj. TriAdj is composed of 10 μg polyphosphazene: 20 μg HDP IDR1002: 10 μg Poly(I:C) weight ratio per dose.

FIG. 3 shows the IgG2a titres as measured by ELISA in the serum of Balb/c mice following intramuscular administration of 50 μg ovalbumin as described in the examples, four weeks after the first immunization and before the second immunization (4 weeks) and eight weeks after the first administration (8 weeks). Data represent the median value and interquartile range (n=6). Dotted bars: antigen only; Grey bars: antigen mixed with PCEP-TriAdj; Black bars: antigen mixed with 37-TriAdj; White bars: antigen mixed with 37-TriAdj. TriAdj is composed of 10 μg polyphosphazene: 20 μg HDP IDR1002: 10 μg Poly(I:C) weight ratio per dose.

FIG. 4 shows the IgG1 titres as measured by ELISA in the serum of Balb/c mice following intramuscular administration of 1 μg ovalbumin as described in the examples, four weeks after the first immunization and before the second immunization (4 weeks) and eight weeks after the first administration (8 weeks). Data represent the median value and interquartile range (n=8). Dotted bars: antigen only; Grey bars: antigen mixed with PCEP-TriAdj; Black bars: antigen mixed with 11-TriAdj; White bars: antigen mixed with 37-TriAdj. TriAdj is composed of 10 μg polyphosphazene: 20 μg HDP IDR1002: 10 μg Poly(I:C) weight ratio per dose.

FIG. 5 shows the IgG2a titres as measured by ELISA in the serum of Balb/c mice following intramuscular administration of 1 μg ovalbumin as described in the examples four weeks after the first immunization and before the second immunization (4 weeks) and eight weeks after the first administration (8 weeks). Data represent the median value and interquartile range (n=8). Dotted bars: antigen only; Grey bars: antigen mixed with PCEP-TriAdj; Black bars: antigen mixed with 11-TriAdj; White bars: antigen mixed with 37-TriAdj. TriAdj is composed of 10 μg polyphosphazene: 20 μg HDP IDR1002: 10 μg Poly(I:C) weight ratio per dose.

FIG. 6 shows the IgG1 titres as measured by ELISA in the serum of balb/c mice following intramuscular administration of 1 μg ovalbumin as described in the examples, four weeks after the first immunization and before the second immunization (4 weeks) and eight weeks after the first administration (8 weeks). Data represent the median value and interquartile range (n=8). Black bars: antigen mixed with 11-TriAdj; Striped bars: antigen mixed with 39-TriAdj; White bars: antigen mixed with 37-TriAdj; Grey bars: antigen mixed with PCEP-TriAdj. TriAdj is composed of 10 μg polyphosphazene: 20 μg HDP IDR1002: 10 μg Poly(I:C) weight ratio per dose.

FIG. 7 shows the IgG2a titres as measured by ELISA in the serum of balb/c mice following intramuscular administration of 1 μg ovalbumin as described in the examples, four weeks after the first immunization and before the second immunization (4 weeks) and eight weeks after the first administration (8 weeks). Data represent the median value and interquartile range (n=8). Black bars: antigen mixed with 11-TriAdj; Striped bars: antigen mixed with 39-TriAdj; White bars: antigen mixed with 37-TriAdj; Grey bars: antigen mixed with PCEP-TriAdj. TriAdj is composed of 10 μg polyphosphazene: 20 μg HDP IDR1002: 10 μg Poly(I:C) weight ratio per dose.

FIG. 8 shows the IgG1 titres as measured by ELISA in the serum of balb/c mice following intramuscular administration of 1 μg ovalbumin as described in the examples, four weeks after the first immunization and before the second immunization (4 weeks) and eight weeks after the first administration (8 weeks). Data represent the median value and interquartile range (n=8). Dotted bars: antigen without adjuvant; Black bars: antigen mixed with 11-TriAdj; Striped bars: antigen mixed with 39-TriAdj; White bars: antigen mixed with 37-TriAdj; Grey bars: antigen mixed with PCEP-TriAdj. TriAdj is composed of 10 μg polyphosphazene: 20 μg HDP IDR1002: 10 μg Poly(I:C) weight ratio per dose.

FIG. 9 shows the IgG2a titres as measured by ELISA in the serum of balb/c mice following intramuscular administration of 1 μg ovalbumin as described in the examples, four weeks after the first immunization and before the second immunization (4 weeks) and eight weeks after the first administration (8 weeks). Data represent the median value and interquartile range (n=8). Dotted bars: antigen without adjuvant; Black bars: antigen mixed with 11-TriAdj; Striped bars: antigen mixed with 39-TriAdj; White bars: antigen mixed with 37-TriAdj; Grey bars: antigen mixed with PCEP-TriAdj. TriAdj is composed of 10 μg polyphosphazene: 20 μg HDP IDR1002: 10 μg Poly(I:C) weight ratio per dose.

FIG. 10 is a schematic diagram depicting an embodiment of a synthetic method for preparing a cyclopolyphosphazene.

FIG. 11 is a schematic diagram depicting an embodiment of a design strategy for one or more ligands of a cyclopolyphosphazene.

FIGS. 12A-E are a list of embodiments of cyclopolyphosphazenes and ligands, or intermediates in the preparation of both.

FIG. 13 is a schematic diagram depicting an embodiment of a synthetic method for preparing an oligomeric cyclopolyphosphazene.

FIG. 14 is a schematic diagram depicting partial to full substitution of a cyclopolyphosphazene.

### DETAILED DESCRIPTION

One or more illustrative embodiments have been described by way of example. Described herein are compositions, methods and uses relating to cyclopolyphosphazenes. It will be appreciated that embodiments and examples are provided for illustrative purposes intended for those skilled in the art, and are not meant to be limiting in any way. All references to embodiments, examples, aspects, formulas, compounds, compositions, solutions, and the like is intended to be illustrative and non-limiting.

The practice of the present invention will employ, unless otherwise indicated, conventional methods of microbiology, chemistry, biochemistry, recombinant DNA techniques and immunology, within the skill of the art. Such techniques are explained fully in the literature. See. e.g., Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company, current Edition); A. L. Lehninger, Biochemistry (Worth Publishers, Inc., current addition); Sambrook, et al., Molecular Cloning: A Laboratory Manual (current addition); Methods In Enzymology (S. Colowick and N. Kaplan eds., Academic Press, Inc.), all of which are herein incorporated by reference in their entirety.

The following amino acid abbreviations are used throughout the text:

| | |
|---|---|
| Alanine: Ala (A) | Arginine: Arg (R) |
| Asparagine: Asn (N) | Aspartic acid: Asp (D) |
| Cysteine: Cys (C) | Glutamine: Gln (Q) |
| Glutamic acid: Glu (E) | Glycine: Gly (G) |
| Histidine: His (H) | Isoleucine: Ile (I) |
| Leucine: Leu (L) | Lysine: Lys (K) |
| Methionine: Met (M) | Phenylalanine: Phe (F) |
| Proline: Pro (P) | Serine: Ser (S) |
| Threonine: Thr (T) | Tryptophan: Trp (W) |
| *Tyrosine: Tyr (Y) | Valine: Val (V) |
| Dehydroalanine (Dha) | Dehydrobutyrine (Dhb) |

Amino acid sequences referred to herein are summarized in Table 1 below.

TABLE 1
Sequences presented herein:

| SEQ | | |
|---|---|---|
| ID | | |
| NO | SEQUENCE | NAME |
| 1 | ILPWKWPWWPWRR | indolicidin |
| 2 | VFLRRIRVIVIR | JK1 |
| 3 | VFWRRIRVWVIR | JK2 |
| 4 | VQLRAIRVRVIR | JK3 |
| 5 | VQLRRIRVWVIR | JK 4 |
| 6 | VQWRAIRVRVIR | JK5 |
| 7 | VQWRRIRVWVIR | JK6 |
| 8 | TCCATGACGTTCCTG | CpG 1826 |
| | ACGTT | |
| 9 | TCGTCGTTGTCGTTT | CpG 2007 |
| | TGTCGTT | |
| 10 | TCGTCGTTTTGTCGT | CpG 7909 |
| | TTTGTCGTT | or 10103 |
| 11 | GGGGACGACGTCGTG | CpG 8954 |
| | GGGGGG | |
| 12 | TCGTCGTTTTCGGCG | CpG 2395 |
| | CGCGCCG | or 10101 |
| 13 | AAAAAAGGTACCTAA | Non-CpG |
| | ATAGTATGTTTCTGA | oligo |
| | AA | |
| 14 | GRFKRFRKKFKKLFK | BMAP27 |
| | KLSPVIPLLHLG | |
| 15 | GGLRSLGRKILRAWK | BMAP28 |
| | KYGPIIVPIIRIG | |
| 16 | RLARIVVIRVAR | Bactenicin 2a |
| | | (Bac2a) |
| 17 | LLGDFFRKSKEKIGK | human LL-37 |
| | EFKRIVQRIKDFLRN | |
| | LVPRTES | |
| 18 | VQLRIRVAVIRA | HH2 |
| 19 | VQRWLIVWRIRK | 1002 |
| 20 | VRLIVAVRIWRR | 1018 |
| 21 | IWVIWRR | HH18 |
| 22 | Ile-Dhb-Ala-Ile- | Nisin Z |
| | Dha-Leu-Ala-Abu- | |
| | Pro-Gly-Ala-Lys- | |
| | Abu-Gly-Ala-Leu- | |
| | Met-Gly-Ala-Asn- | |
| | Met-Lys-Abu-Ala- | |
| | Abu-Ala-Asn-Ala- | |
| | Ser-Ile-Asn-Val- | |
| | Dha-Lys | |
| 23 | V**R*IRV*VIR, | conserved |
| | *= any amino acid | motif |
| 24 | ILKWKWPWWPWRR | HH111 |
| 25 | ILPWKKPWWPWRR | HH113 |
| 26 | ILKWKWPWWKWRR | HH970 |
| 27 | ILRWKWRWWRWRR | HH1010 |

It must be noted that, as used in this specification and the appended claims, the singular forms “a”, “an” and “the” ˜ include plural referents unless the content clearly dictates otherwise. Thus, for example, reference to “a host defense peptide” may include a mixture of two or more host defense peptides, and the like.

Disclosed herein are compounds of formula I:

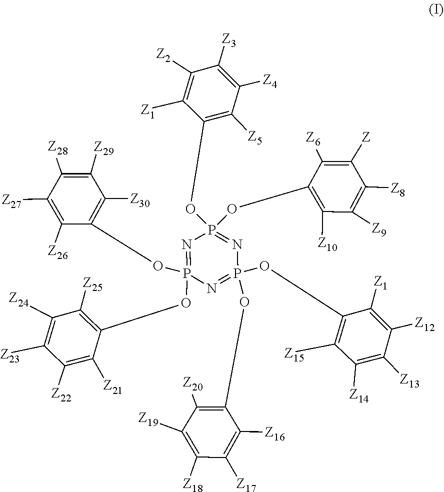

and tautomers, stereoisomers, polymorphs, hydrates, solvates, or pharmaceutically acceptable salts thereof.

In embodiments disclosed herein, each of Z₁, Z₂, Z₃, Z₄, Z₅, Z₆, Z₇, Z₈, Z₉, Z₁₀, Z₁₁, Z₁₂, Z₁₃, Z₁₄, Z₁₅, Z₁₆, Z₁₇, Z₁₈, Z₁₉, Z₂₀, Z₂₁, Z₂₂, Z₂₃, Z₂₄, Z₂₅, Z₂₆, Z₂₇, Z₂₈, Z₂₉, and Z₃₀, hereinto referred to as Z₁₋₃₀, may be independently selected from: H or formula II:

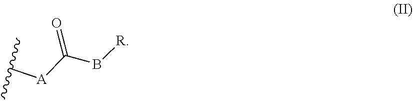

In some embodiments, at least one of Z₁₋₃₀ is substituted with formula II. Each formula II substitution of Z₁₋₃₀ may be identical or non-identical.

It will be understood by a person of skill in the art that “each of Z₁₋₃₀ may be identical or non-identical” or “each of Y₁₋₃₀ may be identical or non-identical” may refer to embodiments in which each of the Z₁₋₃₀ or Y₁₋₃₀ may be represented by non-identical groups of formula II or III. Each A, B and/or R groups may be the same between one or more Z₁₋₃₀ (or Y₁₋₃₀) substitutions or unique from one another. For example, Z₁ may be substituted with formula II wherein the A group is an oxygen atom and Z₁₃ may be substituted with formula II wherein the A group is a nitrogen atom.

In embodiments disclosed herein, one or more A-groups, if present, is selected from C₁-C₇ alkyl, C₂-C₇ alkenyl, C₂-C₇ alkynyl, O, S, and N. C₁-C₇ alkyl, C₂-C₇ alkenyl, and/or C₂-C₇ alkynyl may be straight or branched and optionally substituted by one or more substituents selected from: 1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc).

It will be understood by a person of skill in the art that “if present” refers to groups that are optional within the entire structure. In embodiments in which the group is present, the connectivity of the surrounding groups are as depicted. In embodiments where the group, for example “A” in formula II and III, is absent, the flanking groups would be directly connected to one another. In such cases, the carbonyl of formula II or III would be directly connected to the appropriate Z-group position, such as Z₁.

In embodiments disclosed herein, one or more B-groups may be selected from C₁-C₇ alkyl, C₂-C₇ alkenyl, C₂-C₇ alkynyl, H, O, S, and N, wherein C₁-C₇ alkyl, C₂-C₇ alkenyl, and/or C₂-C₇ alkynyl are straight or branched and optionally substituted by one or more substituents selected from: 1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc).

In embodiments disclosed herein, one or more R-groups (sometimes referred to herein as “ligands”), if present, is selected from H, C₁-C₄₅ alkyl, C₂-C₄₅ alkenyl, and C₂-C₄₅ alkynyl, wherein C₁-C₄₅ alkyl, C₂-C₄₅ alkenyl, and/or C₂-C₄₅ alkynyl are straight or branched and optionally substituted. The one or more R-groups may comprise one or more substituents selected from: 1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc). In some embodiments described herein, R-groups may be selected from Table 2.

TABLE 2
Examples of possible R-groups (ligands).

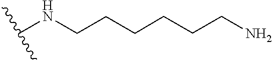 [SMILES: *NCCCCCCN]

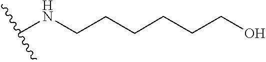 [SMILES: *NCCCCCCO]

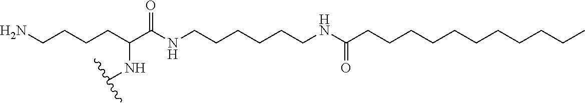 [SMILES: *NC(CCCCN)C(=O)NCCCCCCNC(=O)CCCCCCCCCCC]

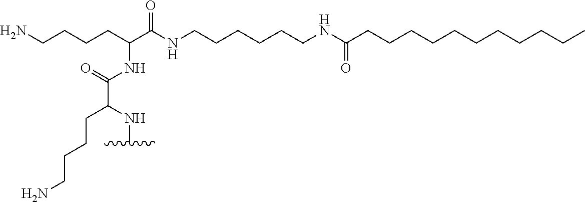 [SMILES: *NC(CCCCN)C(=O)NC(CCCCN)C(=O)NCCCCCCNC(=O)CCCCCCCCCCC]

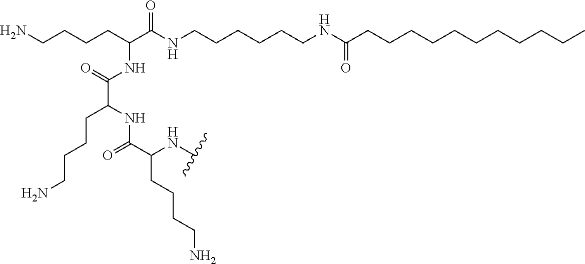 [SMILES: *NC(CCCCN)C(=O)NC(CCCCN)C(=O)NC(CCCCN)C(=O)NCCCCCCNC(=O)CCCCCCCCCCC]

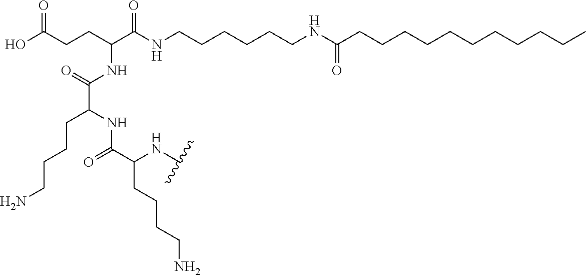 [SMILES: *NC(CCCCN)C(=O)NC(CCCCN)C(=O)NC(CCC(=O)O)C(=O)NCCCCCCNC(=O)CCCCCCCCCCC]

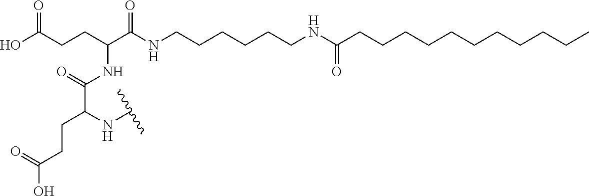 [SMILES: *NC(CCC(=O)O)C(=O)NC(CCC(=O)O)C(=O)NCCCCCCNC(=O)CCCCCCCCCCC]

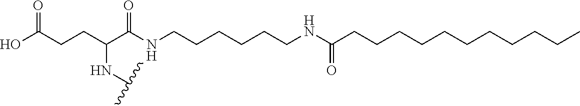 [SMILES: *NC(CCC(=O)O)C(=O)NCCCCCCNC(=O)CCCCCCCCCCC]

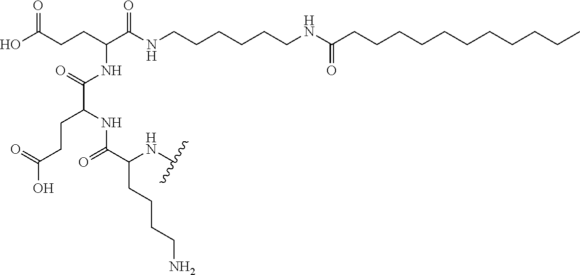 [SMILES: *NC(CCCCN)C(=O)NC(CCC(=O)O)C(=O)NC(CCC(=O)O)C(=O)NCCCCCCNC(=O)CCCCCCCCCCC]

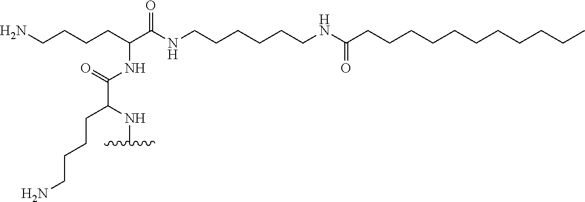 [SMILES: *NC(CCCCN)C(=O)NC(CCCCN)C(=O)NCCCCCCNC(=O)CCCCCCCCCCC]

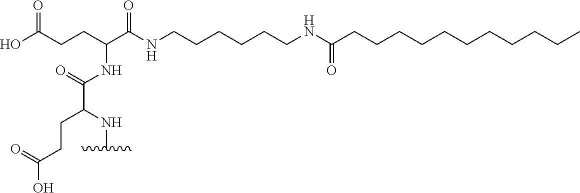 [SMILES: *NC(CCC(=O)O)C(=O)NC(CCC(=O)O)C(=O)NCCCCCCNC(=O)CCCCCCCCCCC]

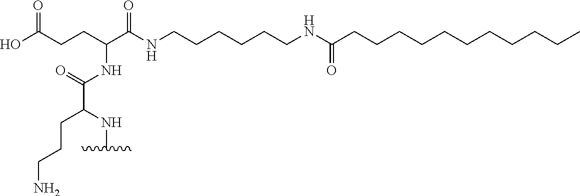 [SMILES: *NC(CCCN)C(=O)NC(CCC(=O)O)C(=O)NCCCCCCNC(=O)CCCCCCCCCCC]

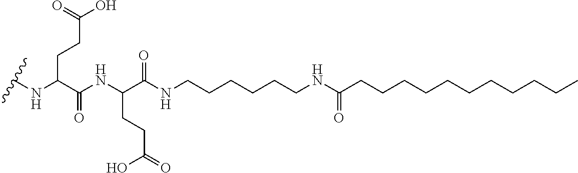 [SMILES: *NC(CCC(=O)O)C(=O)NC(CCC(=O)O)C(=O)NCCCCCCNC(=O)CCCCCCCCCCC]

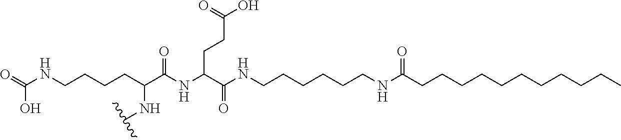 [SMILES: *NC(CCCCNC(=O)O)C(=O)NC(CCC(=O)O)C(=O)NCCCCCCNC(=O)CCCCCCCCCCC]

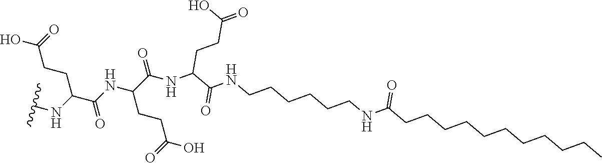 [SMILES: *NC(CCC(=O)O)C(=O)NC(CCC(=O)O)C(=O)NC(CCC(=O)O)C(=O)NCCCCCCNC(=O)CCCCCCCCCCC]

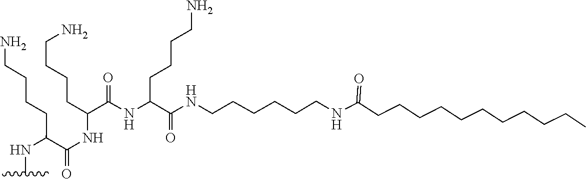 [SMILES: *NC(CCCCN)C(=O)NC(CCCCN)C(=O)NC(CCCCN)C(=O)NCCCCCCNC(=O)CCCCCCCCCCC]

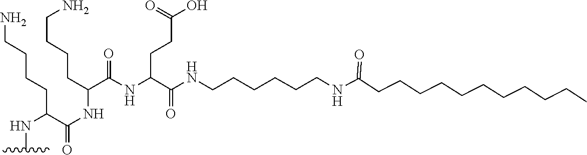 [SMILES: *NC(CCCCN)C(=O)NC(CCCCN)C(=O)NC(CCC(=O)O)C(=O)NCCCCCCNC(=O)CCCCCCCCCCC]

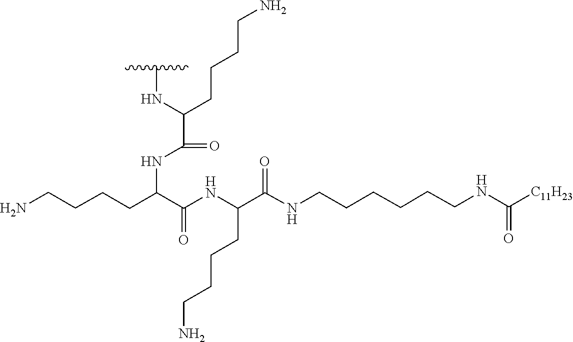

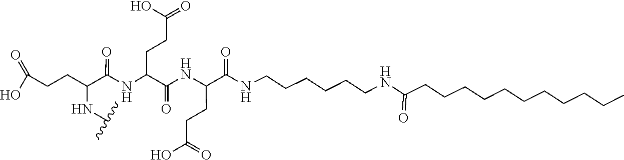 [SMILES: *NC(CCC(=O)O)C(=O)NC(CCC(=O)O)C(=O)NC(CCC(=O)O)C(=O)NCCCCCCNC(=O)CCCCCCCCCCC]

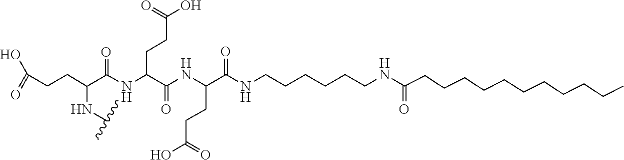 [SMILES: *NC(CCC(=O)O)C(=O)NC(CCC(=O)O)C(=O)NC(CCC(=O)O)C(=O)NCCCCCCNC(=O)CCCCCCCCCCC]

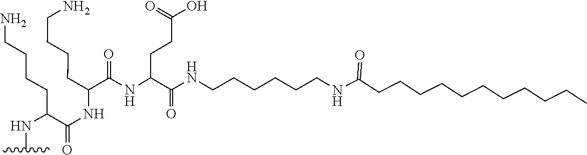 [SMILES: *NC(CCCCN)C(=O)NC(CCCCN)C(=O)NC(CCC(=O)O)C(=O)NCCCCCCNC(=O)CCCCCCCCCCC]

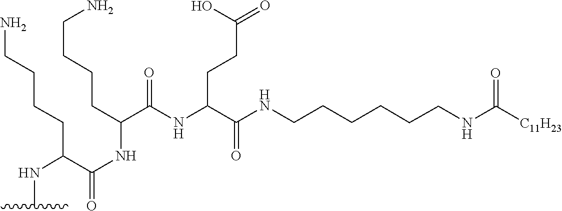

In embodiments disclosed herein, cyclopolyphosphazenes may be in various tautomeric forms, stereoisomers, polymorphs, hydrates, solvates, or pharmaceutically acceptable salts thereof.

In embodiments disclosed herein, oligomeric structures may comprise two or more cyclopolyphosphazenes compounds as defined herein. It will be understood by a person of skill in the art that “oligomeric” may refer to repeating units of either identical or non-identical cyclopolyphosphazenes that are covalently linked.

Embodiments where two identical cyclopolyphosphazenes are linked may be referred to as a homodimer. Embodiments where three non-identical cyclopolyphosphazenes are linked may be referred to as a heterotrimer. Oligomeric structures may comprise two or more units, including 3, 4, 5, 6, 7, 8, 9, 10, 11, 12, 13, 14, 15 or greater units.

As disclosed herein, the two or more compounds in an oligomeric structure may be linked via a suitable connection. In some cases, the suitable connection is an amide or ester bond. The amide or ester bond may be formed via an activated ester, such as an N-hydroxy succinimide ester, and a nucleophilic group, such as a free hydroxyl or amine group. In some cases, each cyclopolyphosphazene unit in the oligomeric structure comprises an activated ester and a nucleophilic group for self-assembly into large oligomeric structures.

Disclosed herein are adjuvant compositions comprising one or more cyclopolyphosphazene compounds as defined herein, such as compound 37 or 39 and one or more of: a host defense peptide, an immunostimulatory sequence and a pharmaceutically acceptable excipient, diluent or carrier. Embodiments of the adjuvant compositions may comprise IDR-1002 (SEQ ID NO:19) as the host defense peptide. In some cases, the immunostimulatory sequence is poly(I:C). Embodiments of the adjuvant composition may further comprise an antigen. The antigen may be derived from a virus, bacteria, parasite, prion or fungus.

Disclosed herein are compositions comprising adjuvant compositions as described herein and a pharmaceutically acceptable excipient.

Methods of enhancing an immune response to a selected antigen are disclosed herein. The methods may comprise administering to a subject the embodiments of the adjuvant compositions, which may comprise a pharmaceutically acceptable excipient.

The adjuvant compositions are useful for the prevention and treatment of infectious diseases in humans and other animals, caused by a variety of pathogens that invade the mucosa and other parts of the body, including diseases caused by bacteria, mycobacteria, viruses, fungi, prions, parasites and the like, when used with a co-administered antigen.

The compositions and methods described herein can be used with one or multiple antigens or immunogens including polypeptide, polynucleotide, polysaccharide, or lipid antigens or immunogens, as well as with inactivated or attenuated pathogens, to produce an immune response, such as a systemic or mucosal immune response, in the subject to which the systems are delivered. The immune response can serve to protect against future infection or lessen or ameliorate the effects of infection.

The compositions used herein may be used as immunomodulators. It will be understood by a person of skill in the art that immunomodulators may affect the immune system when administered. Immunomodulators may be used to protect a subject against pathogens when administered prior to viral infection/challenge, for example 24 h. Such compositions may not comprise antigens. Immunomodulators are known in the art, see e.g. Martinez E C, Intranasal treatment with a novel immunomodulator mediates innate immune protection against lethal pneumonia virus of mice. Antiviral Res. 135 (2016) 108-119.

It will be understood that by a person of skill in the art that “host defense peptide” or “HDP” is meant any of the various host defense peptides that have the ability to enhance an immune response to a co-administered antigen. The DNA and corresponding amino acid sequences for various host defense peptides are known and described in detail below. Host defense peptides for use in the present methods include the full-length (i.e., a prepro sequence if present, the entire prepro molecule) or substantially full-length proteins, as well as biologically active fragments, fusions or mutants of the proteins. The term also includes postexpression modifications of the polypeptide, for example, glycosylation, acetylation, phosphorylation and the like. Furthermore, for purposes of the present invention, a “host defense peptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification. It is readily apparent that the host defense peptides may therefore comprise an entire leader sequence, the mature sequence, fragments, truncated and partial sequences, as well as analogs, muteins and precursor forms of the molecule. The term also intends deletions, additions and substitutions to the reference sequence, so long as the molecule retains the desired biological activity.

It will be understood that by a person of skill in the art that “poly(I:C) oligonucleotide” or “poly(I:C)” is a synthetic viral-like mis-matched double-stranded immunostimulatory ribonucleic acid containing strands of polyriboinosinic acid and polyribocytidylic acid that are held together by hydrogen bonds between purine and pyrimidine bases in the chains. Poly(I:C) has been found to have a strong interferon-inducing effect in vitro and is therefore of significant interest in infectious disease research.

It will be understood that by a person of skill in the art that “CpG oligonucleotide” or “CpG ODN” is an immunostimulatory nucleic acid containing at least one cytosine-guanine dinucleotide sequence (i.e., a 5′ cytidine followed by 3′ guanosine and linked by a phosphate bond) and which activates the immune system. An “unmethylated CpG oligonucleotide” is a nucleic acid molecule which contains an unmethylated cytosine-guanine dinucleotide sequence (i.e., an unmethylated 5′ cytidine followed by 3′ guanosine and linked by a phosphate bond) and which activates the immune system. A “methylated CpG oligonucleotide” is a nucleic acid which contains a methylated cytosine-guanine dinucleotide sequence (i.e., a methylated 5′ cytidine followed by a 3′ guanosine and linked by a phosphate bond) and which activates the immune system. CpG oligonucleotides are well known in the art and described in, e.g., U.S. Pat. Nos. 6,194,388; 6,207,646; 6,214,806; 6,218,371; 6,239,116; and 6,339,068; PCT Publication No. WO 01/22990; PCT Publication No. WO 03/015711; US Publication No. 20030139364, which patents and publications are incorporated herein by reference in their entireties.

It will be understood that by a person of skill in the art that “polyphosphazene” is a cyclic or acyclic (unless otherwise specified), high-molecular weight, water-soluble polymer, containing a backbone of alternating phosphorous and nitrogen atoms and organic side groups or ligands attached at each phosphorus atom. See. e.g., Payne et al., Vaccine (1998) 16:92-98; Andrianov & Payne, Adv. Drug. Deliv. Rev. (1998) 31:185-196.

It will be understood that by a person of skill in the art that “antigen” or “immunogen” is a molecule, which contains one or more epitopes (defined below) that will stimulate a host's immune system to make a cellular antigen-specific immune response when the antigen is presented, and/or a humoral antibody response. The terms denote both subunit antigens, i.e., proteins which are separate and discrete from a whole organism with which the antigen is associated in nature, as well as killed, attenuated or inactivated bacteria, viruses, parasites or other microbes. Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide that expresses a therapeutic or immunogenic protein, or antigenic determinant in vivo, such as in gene therapy and nucleic acid immunization applications, is also included in the definition of antigen herein. Further, for purposes of the present invention, antigens can be derived from any of several known viruses, bacteria, parasites, prions, and fungi, as well as any of the various tumor antigens.

The term “derived from” is used to identify the original source of a molecule (e.g., bovine or human) but is not meant to limit the method by which the molecule is made which can be, for example, by chemical synthesis or recombinant means.

The terms “analog” and “mutein” may refer to biologically active derivatives of the reference molecule that retain desired activity as described herein. In general, the term “analog” refers to compounds having a native polypeptide sequence and structure with one or more amino acid additions, substitutions (generally conservative in nature) and/or deletions, relative to the native molecule, so long as the modifications do not destroy activity and which are “substantially homologous” to the reference molecule as defined below. The term “mutein” refers to peptides having one or more peptide mimics (“peptoids”), such as those described in International Publication No. WO 91/04282. Preferably, the analog or mutein has at least the same desired activity as the native molecule. Methods for making polypeptide analogs and muteins are known in the art and are described further below.

The terms also encompass purposeful mutations that are made to the reference molecule. Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3)non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the molecule of interest may include up to about 5-10 conservative or non-conservative amino acid substitutions, or even up to about 15-20 conservative or non-conservative amino acid substitutions, or any integer between 5-20, so long as the desired function of the molecule remains intact. One of skill in the art can readily determine regions of the molecule of interest that can tolerate change by reference to Hopp/Woods and Kyte-Doolittle plots, well known in the art.

It will be understood that by a person of skill in the art that “fragment” is a molecule consisting of only a part of the intact full-length polypeptide sequence and structure. The fragment can include a C-terminal deletion, an N-terminal deletion, and/or an internal deletion of the native polypeptide. A fragment will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence, provided that the fragment in question retains the ability to elicit the desired biological response.

It will be understood that by a person of skill in the art that “immunogenic fragment” is a fragment of a parent molecule which includes one or more epitopes and thus can modulate an immune response or can act as an adjuvant for a co-administered antigen and/or is capable of inducing an adaptive immune response. Such fragments can be identified using any number of epitope mapping techniques, well known in the art. See. e.g., Epitope Mapping Protocols in Methods in Molecular Biology, Vol. 66 (Glenn E. Morris, Ed., 1996) Humana Press, Totowa, New Jersey, incorporated herein by reference. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 81:3998-4002; Geysen et al., (1986)Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See. e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., J. Mol. Biol. (1982) 157:105-132 for hydropathy plots.

Immunogenic fragments, for purposes of the present invention, will usually be at least about 2 amino acids in length, more preferably about 5 amino acids in length, and most preferably at least about 10 to 15 amino acids in length. There is no critical upper limit to the length of the fragment, which could comprise nearly the full-length of the protein sequence, or even a fusion protein comprising two or more epitopes of the protein in question.

It will be understood that by a person of skill in the art that the term “epitope” refers to the site on an antigen or hapten to which specific B cells and T cells respond. The term is also used interchangeably with “antigenic determinant” or “antigenic determinant site.” Antibodies that recognize the same epitope can be identified in a simple immunoassay showing the ability of one antibody to block the binding of another antibody to a target antigen.

An “immunological response” or “immune response” to a composition is the development in the host of a cellular and/or antibody-mediated immune response to the composition or vaccine of interest. Usually, an “immunological response” includes but is not limited to one or more of the following effects: the production of antibodies, B cells, helper T cells, suppressor T cells, and/or cytotoxic T cells and/or γδ T cells, directed specifically to an antigen or antigens included in the composition or vaccine of interest. Preferably, the host will display a protective immunological response to the microorganism in question, e.g., the host will be protected from subsequent infection by the pathogen and such protection will be demonstrated by either a reduction or lack of symptoms normally displayed by an infected host or a quicker recovery time.

It will be understood that by a person of skill in the art that the term “immunogenic” molecule refers to a molecule which elicits an immunological response as described above. An “immunogenic” protein or polypeptide, as used herein, includes the full-length sequence of the protein in question, including the precursor and mature forms, analogs thereof, or immunogenic fragments thereof.

An “immunological response” to a composition is the development in the host of a cellular and/or antibody-mediated immune response to the composition or vaccine of interest. Usually, an “immunological response” includes but is not limited to one or more of the following effects: the production of antibodies, B cells, helper T cells, suppressor T cells, and/or cytotoxic T cells and/or γδ T cells, directed specifically to an antigen or antigens included in the composition or vaccine of interest. Preferably, the host will display a protective immunological response to the microorganism in question, e.g., the host will be protected from subsequent infection by the pathogen and such protection will be demonstrated by either a reduction or lack of symptoms normally displayed by an infected host or a quicker recovery time.

An adjuvant composition comprising a host defense peptide, a polyphosphazene and an immunostimulatory sequence “enhances” or “increases” the immune response, or displays “enhanced” or “increased” immunogenicity vis-a-vis a selected antigen when it possesses a greater capacity to elicit an immune response than the immune response elicited by an equivalent amount of the antigen when delivered without the adjuvant composition. Such enhanced immunogenicity can be determined by administering the antigen and adjuvant composition, and antigen controls to animals and comparing antibody titers against the two using standard assays such as radioimmunoassay and ELISAs, well known in the art.

It will be understood that by a person of skill in the art that “substantially purified” may generally refer to isolation of a substance (compound, polynucleotide, protein, polypeptide, polypeptide composition) such that the substance comprises the majority percent of the sample in which it resides. Typically in a sample, a substantially purified component comprises 50%, preferably 80/0-85/0, more preferably 90-95% of the sample. Techniques for purifying polynucleotides and polypeptides of interest are well-known in the art and include, for example, ion-exchange chromatography, affinity chromatography, metal chelation chromatography, reversed phase chromatography, hydrophobic interaction chromatography, and sedimentation according to density.

It will be understood that by a person of skill in the art that “isolated” may mean that the indicated molecule is separate and discrete from the whole organism with which the molecule is found in nature or is present in the substantial absence of other biological macro-molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

It will be understood that by a person of skill in the art that “homology” refers to the percent identity between two polynucleotide or two polypeptide moieties. Two nucleic acid, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified sequence.

It will be understood that by a person of skill in the art that “identity” refers to an exact nucleotide-to-nucleotide or amino acid-to-amino acid correspondence of two polynucleotides or polypeptide sequences, respectively. Percent identity can be determined by a direct comparison of the sequence information between two molecules (the reference sequence and a sequence with unknown % identity to the reference sequence) by aligning the sequences, counting the exact number of matches between the two aligned sequences, dividing by the length of the reference sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, DC, incorporated herein by reference, which adapts the local homology algorithm of Smith and Waterman Advances in Appl. Math. 2:482-489, 1981 for peptide analysis, incorporated herein by reference. Programs for determining nucleotide sequence identity are available in the Wisconsin Sequence Analysis Package, Version 8 (available from Genetics Computer Group, Madison, WI) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, CA). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See. e.g., Sambrook et al., supra; Nucleic Acid Hybridization (B. D. Hames & S. J. Higgns eds. 1984), both of which are incorporated herein by reference.

It will be understood that by a person of skill in the art that “recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

It will be understood that by a person of skill in the art that the terms “effective amount” or “pharmaceutically effective amount” of a composition, or a component of the composition, refers to a nontoxic but sufficient amount of the composition or component to provide the desired response, such as enhanced immunogenicity, and, optionally, a corresponding therapeutic effect. The exact amount required will vary from subject to subject, depending on the species, age, and general condition of the subject, the severity of the condition being treated, and the particular components of interest, mode of administration, and the like. An appropriate “effective” amount in any individual case may be determined by one of ordinary skill in the art using routine experimentation.

It will be understood that by a person of skill in the art that “vertebrate subject” is any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The invention described herein is intended for use in any of the above vertebrate species, since the immune systems of all of these vertebrates operate similarly.

It will be understood that by a person of skill in the art that term “treatment” as used herein refers to either (1) the prevention of infection or reinfection (prophylaxis), or (2) the reduction or elimination of symptoms of the disease of interest (therapy).

It will be understood to a person of skill in the art that any of the functional groups or substitutions described herein may be protected by a suitable protecting group (PG). For example, amines may be protected by a t-butyl carbamate (Boc) group. Other examples of protecting groups include: 9-Fluorenylmethyl carbamate (Fmoc), benzyl carbamate (Cbz), acyl, trifluoroacyl, phthalimide, benzyl (Bn), p-toluenesulfonamide, dithiane, acetal (cyclic or acyclic), hydrazone, alkyl or aryl esters, allyl, methoxymethyl ether (MOM ether), alkyl silyl groups (such as TBDMS and others), tetrahydropyranyl (THP) and others known in the art. Protecting groups may be removed via suitable deprotection conditions known in the art.

It will be understood by a person of skill in the art that C₁-C₇ alkyl or C₁-C₄₅ alkyl, in these embodiments as referred to herein may refer to an alkyl group between one and seven carbons. This may be understood to include straight or branched alkyl groups including for example: methyl, ethyl, isopropyl, n-propyl, tert-butyl, n-butyl, sec-butyl and others. Alkyl chains greater than seven carbons are also contemplated, for example alkyl chains of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 and greater carbons.

It will be understood by a person of skill in the art that C₂-C₇ alkenyl or C₂-C₄₅ alkenyl in these embodiments as referred to herein may refer to an alkyl group between one and seven carbons comprising at least one double bond. This may be understood to include straight or branched alkenyl groups comprising at least one double bond. C₁-C₇ alkenyl may refer chains with two or more double bonds in conjugation. Alkenyl chains greater than seven carbons are also contemplated, for example alkenyl chains of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 and greater carbons.

It will be understood by a person of skill in the art that C₂-C₇ alkynyl or C₂-C₄₅ alkynyl in these embodiments as referred to herein may refer to an alkyl group between one and seven carbons comprising at least one triple bond. This may be understood to include straight or branched alkynyl groups comprising at least one triple bond. C₁-C₇ alkynyl may refer chains with two or more triple bonds in conjugation. Alkynyl chains greater than seven carbons are also contemplated, for example alkynyl chains of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 and greater carbons.

The C₁-C₇ alkyl, C₁-C₄₅ alkyl, C₂-C₇ alkenyl, C₂-C₄₅ alkenyl, C₂-C₇ alkynyl, and/or C₂-C₄₅ alkynyl may be substituted by one or more suitable substituents. For example, each carbon of the one or more C₁-C₇ alkyl, C₁-C₃₀ alkyl, C₂-C₇ alkenyl, C₂-C₃₀ alkenyl, C₂-C₇ alkynyl, and/or C₂-C₃₀ alkynyl chain may be substituted with one or more of: hydroxyl, aldehyde, 1° amino, 2° amino, 3° amino, tert-butyloxycarbonyl (—NH—Boc), cyano, amide, aryl, alkoxy, acetal, ketone, ester, acyl, ether, thioether, thioester, thiol, disulfide, peroxide, imine, imide, oxime, acyl halide, nitro, nitrile, epoxide, sulfonic acid, sulphonamidyl, carbamimidoyl, azide and others.

It will be understood by a person of skill in the art that sulfonamidyl may be understood as a sulfonamide group connected to the parent group, with a nitrogen optionally substituted by 0-2 suitable substituents, such as an alkyl, aryl and others.

It will be understood by a person of skill in the art that a primary (1°), secondary (2°), tertiary (3°), and quaternary (4°) amino groups may refer to an amine with 0, 1, 2 and 3 additional substituents (apart from the parent group), respectfully. Substituents may be alkyl, alkenyl, alkynyl, aryl and others without departing from what is contemplated by the invention. It will be understood by a person of skill in the art that an amide as listed in the claims may be part of the backbone of the alkyl chain or a substituent thereof. Amides may be primary (1°), secondary (2°), or tertiary (3°).

It will be understood by a person of skill in the art that an aryl group may refer to any suitable aromatic ring or rings, such as aromatic hydrocarbons and heterocyclic rings. Examples include benzene (phenyl), benzyl, naphthalene (naphthyl), anthracene (anthracenyl), pyrene (pyrenyl), indene, biphenyl, phenanthrene, pyridine, imidazole, furan, picolinyl, azole, morphorline (morpholinyl), benzathiazole, thiazole and others.

It will be understood by a person of skill in the art that an alkoxy group refers to an ether bond comprising an alkyl group, such as a C₁-C₄₅ alkyl, C₁-C₄₅ alkenyl, C₁-C₄₅ alkylyl chain, connected to the parent group. Ether bonds may connect other non-alkyl groups, such as an aryl group, to a parent group.

It will be understood by a person of skill in the art that an acetal may refer to two geminal alkoxy groups. A hemiacetal will be understood as an alkoxy group connected at the same carbon as a hydroxyl group.

It will be understood by a person of skill in the art that an acyl or alkanoyl group may refer to an alkyl or aryl group connected to a parent group via a ketone. Acyl halide may refer to acyl group that comprises a carbonyl bonded to a halide, such as acyl chloride or acyl bromide.

It will be understood by a person of skill in the art that a halo group or halide may refer to any suitable halogen, for example fluorine (F), bromine (Br), iodine (I) and others.

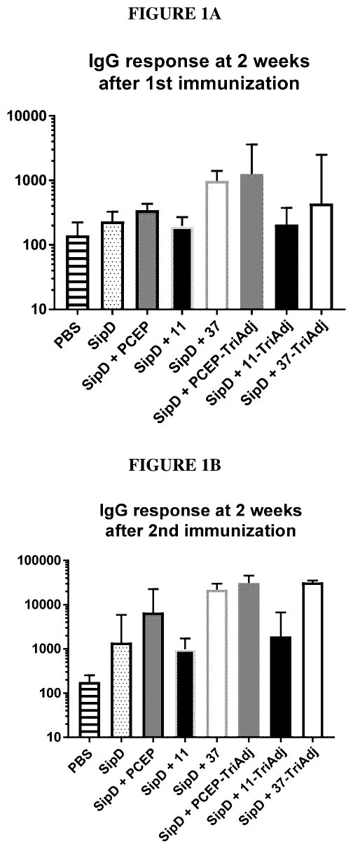

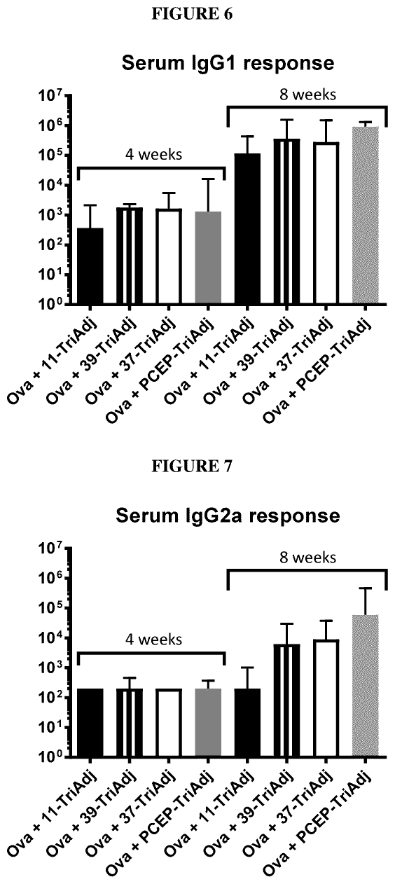

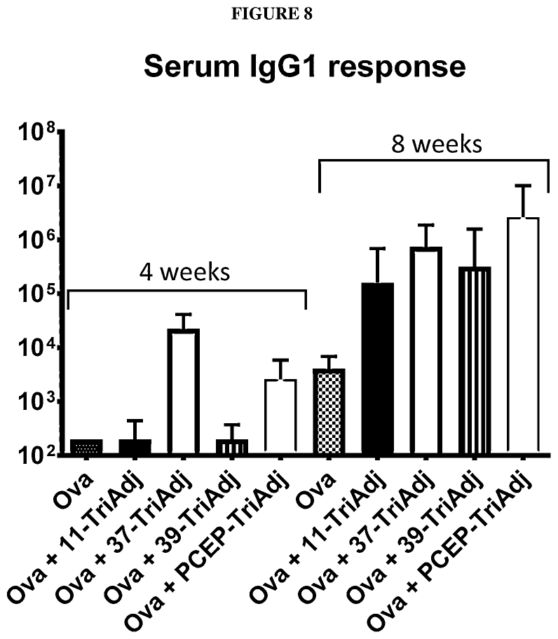

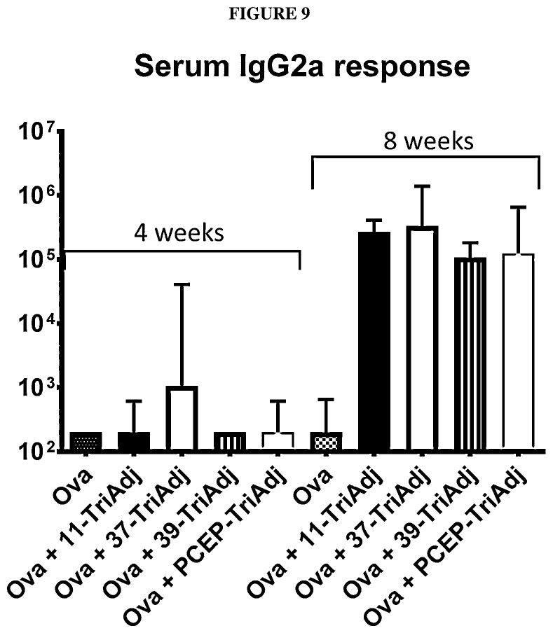

Exemplary polyphosphazene polymers and cyclopolyphosphazenes for use in the present methods and adjuvant compositions are shown in FIGS. 1-9 and include embodiments of the present invention, as well as poly[di(sodium carboxylatophenoxy)phosphazene] (PCPP) and poly(di-4-oxyphenylproprionate)phosphazene (PCEP), in various forms, such as the sodium salt, or acidic forms. Polymer embodiments may be composed of varying percentages of PCPP or PCEP copolymer with hydroxyl groups, such as 90:10 PCPP/OH.

Methods for synthesizing these compounds are known and described in the patents referenced above, as well as in Andrianov et al., Biomacromolecules (2004) 5:1999; Andrianov et al., Macromolecules (2004) 37:414; Mutwiri et al., Vaccine (2007) 25:1204; and in U.S. Pat. Nos. 9,408,908 and 9,061,001, each of which is incorporated herein by reference in its entirety.

Typical amounts of polyphosphazene present in the adjuvant compositions will represent from about 0.01 to about 2500 μg/kg, typically from about 0.05 to about 500 μg/kg, such as from 0.5 to 100 μg/kg, or 1 to 50 μg/kg, or any amount within these values. One of skill in the art can determine the amount of polyphosphazene, as well as the ratio of polyphosphazene to the other components in the adjuvant composition.

In some embodiments, there is provided herein a pharmaceutical composition comprising any one or more of the compounds as described herein, and optionally further comprising a pharmaceutically acceptable excipient, diluent, or carrier. Examples of such pharmaceutically acceptable excipients, diluents, and carriers may be found in Remington: The Science and Practice of Pharmacy (2012). As well, examples of pharmaceutically acceptable carriers, diluents, and excipients may be found in, for example, Remington's Pharmaceutical Sciences (2000-20th edition) and in the United States Pharmacopeia: The National Formulary (USP 24 NF19) published in 1999, each of which are herein incorporated by reference in their entireties. In certain embodiments, a pharmaceutically acceptable carrier, diluent, or excipient may include any suitable carrier, diluent, or excipient known to the person of skill in the art. Examples of pharmaceutically acceptable excipients may include, but are not limited to, cellulose derivatives, sucrose, and starch. The person of skill in the art will recognize that pharmaceutically acceptable excipients may include suitable fillers, binders, lubricants, buffers, glidants, and disintegrants known in the art (see, for example, Remington: The Science and Practice of Pharmacy (2012)). Examples of pharmaceutically acceptable carriers, diluents, and excipients may be found in, for example, Remington's Pharmaceutical Sciences (2000—20th edition) and in the United States Pharmacopeia: The National Formulary (USP 24 NF19) published in 1999.

Intranasal formulations will usually include pharmaceutically acceptable excipients that neither cause major irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa. Agents can be delivered intranasally using nasal drops, sprays, gels, suspensions and emulsions, an inhaler and/or an atomizer. Thus, the intranasal formulation may be administered by methods such as inhalation, spraying, liquid stream lavage, nebulizing, or nasal irrigation. The administering may be to the sinus cavity or the lungs.

For suppositories, the excipients will include traditional binders and carriers, such as, polyalkaline glycols, or triglycerides. Such suppositories may be formed from mixtures containing the active ingredient in the range of about 0.5% to about 10% (w/w), preferably about 1% to about 2%.

Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Aerosol delivery systems typically employ nebulizers and other inhaler devices and systems. Delivering drugs by inhalation requires a formulation that can be successfully aerosolized and a delivery system that produces a useful aerosol of the drug. The particles or droplets should be of sufficient size and mass to be carried to the distal lung or deposited on proximal airways to give rise to a therapeutic effect.

Vaccination is achieved in a single dose or repeated as necessary at intervals, as can be determined readily by one skilled in the art. For example, a priming dose can be followed by one or more booster doses at weekly, monthly, or longer intervals. An appropriate dose depends on various parameters including the recipient (e.g., adult or infant), the particular vaccine antigen, the route and frequency of administration, and the desired effect (e.g., protection and/or treatment), as can be determined by one skilled in the art.

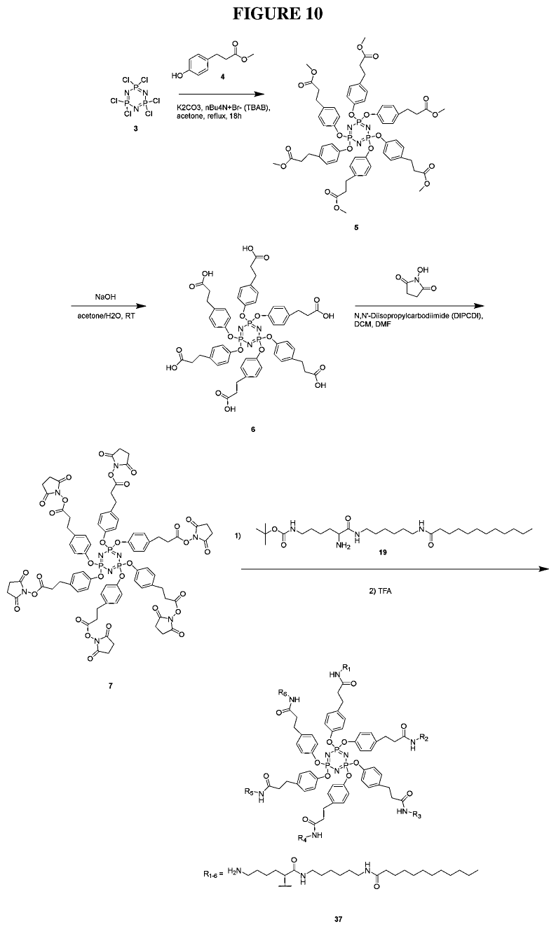

Referring to FIG. 10, cyclopolyphosphazenes may be prepared by a suitable method. Embodiments of the methods of preparation as disclosed herein may be used to synthesize a compound of formula VI:

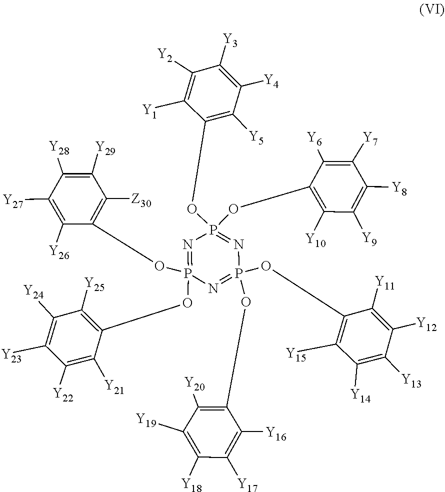

wherein each of Y₁₋₃₀ is independently selected from H and formula (III):

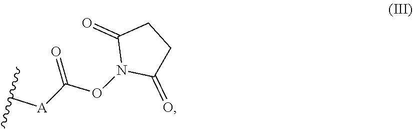

wherein at least one of Y₁₋₃₀ is substituted with formula III and each formula III substitution of Y₁₋₃₀ is identical or non-identical.

The synthetic preparation of cyclopolyphosphazene 37 is merely shown for illustrative purposes. For example, compound 4 may comprise a different alkyl chain with various substituents to vary the A-group of the desired final polyphosphazene compound. Solvents shown in the various steps may be varied without departing from the invention. One or more steps as detailed herein may be combined in a single step.

In a first step, a compound of formula IX:

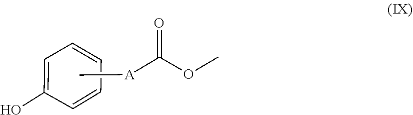

such as compound 4, may be deprotonated by a base, such as potassium carbonate and used as a nucleophile in a first substitution reaction with compound 3 to yield the substituted compound 5. It will be understood by a person of skill in the art that various reagents may be changed without departing from the scope of the invention. For example, the base may be changed to another suitable base, such as triethylamine (TEA), Hunig's base or others. A phase transfer agent such as TBAF or TBAB may be used in stoichiometric or catalytic quantities. It is also contemplated that fewer than six equivalents of the nucleophile 4 may be used to synthesize compounds with less than 6 substituents, for example a penta- or tri-substituted compound using 5 or 3 equivalents, respectively.

In a second step, a compound of formula X:

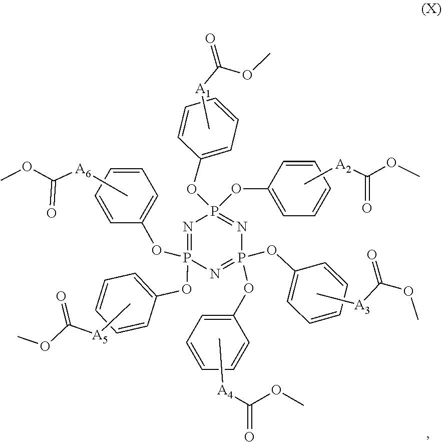

such as the intermediate 5, may be hydrolyzed in the presence of a strong base or hydroxide salt, such as sodium hydroxide or potassium hydroxide, to yield an intermediate of formula XI:

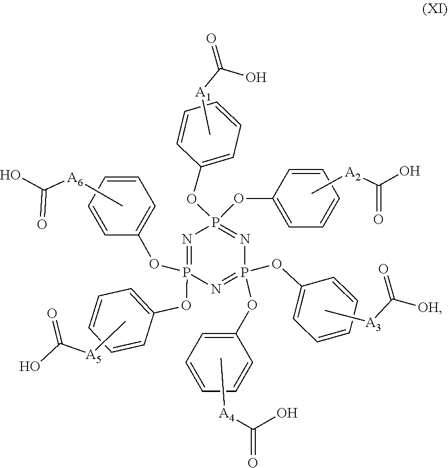

such as acid 6. It will be understood by a person of skill in the art that hydrolysis of intermediate 5 to the acid 6 may occur by various methods known in the art, such as acid or base.

In a third step, a compound of formula XI, such as the intermediate 6, may be activated in a second substitution reaction, using a suitable coupling agent to yield a compound of formula VI:

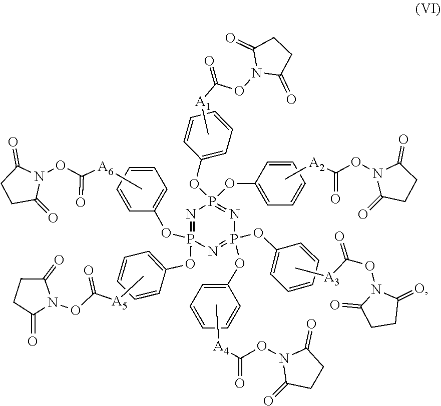

such as the activated ester 7. As depicted in FIG. 10, a suitable coupling agent, such as N,N′-Diisopropylcarbodiimide (DIPCADI), may be used to convert the carboxylic acid group to an activated ester, such as atN-hydroxy succinimde ester. A person of skill in the art will understand that various coupling agents in the art may be used, such as 1-Ethyl-3-(3-dimethylaminopropyl)carbodiimide (EDCI) and others. Various activated esters may be used without departing from the scope of the invention, such as 1-hydroxybenzotriazole, and others. Coupling agents and activating agents are often used in the synthesis of peptides, either solution or solid-phase.

In a fourth step, the activated ester 7 may be coupled to a C₂-C₄₅ nucleophile, such as compound 19, in a third substitution reaction to yield compound 37. The nucleophile may have protecting groups, such as a Boc, t-butyl ester and others, to prevent unwanted reactions. The substitution may also comprise acid, such as trifluoroacidic acid (TFA). The acid may act to remove the protecting groups. The nucleophile, in this case compound 19, may be varied to yield a different product with different substituents. Examples of suitable nucleophiles are provided in Table 3. The reaction may comprise a mix of two or more nucleophiles to yield a mixed product with two or more non-identical substituents. Such reactions may occur in one-pot or step-wise, with or without purification.

TABLE 3
Examples of C2-C45 nucleophiles

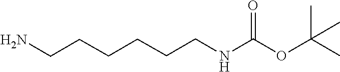

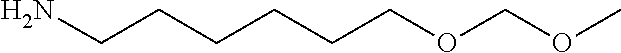

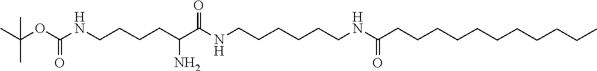 [SMILES: CCCCCCCCCCCC(=O)NCCCCCCNC(=O)C(N)CCCCNC(=O)OC(C)(C)C]

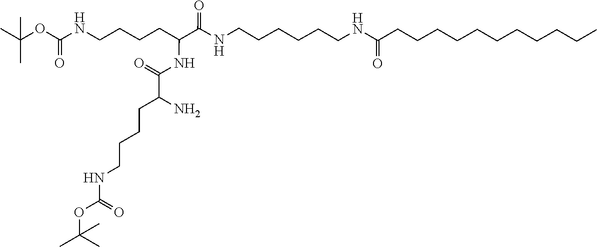 [SMILES: CCCCCCCCCCCC(=O)NCCCCCCNC(=O)C(CCCCNC(=O)OC(C)(C)C)NC(=O)C(N)CCCCNC(=O)OC(C)(C)C]

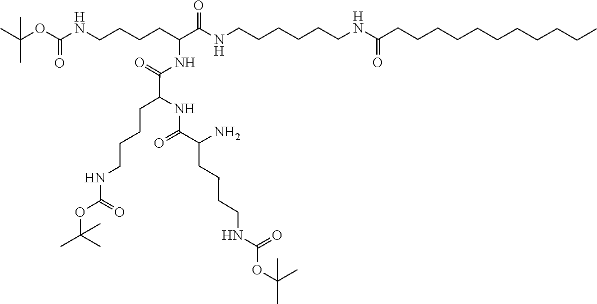 [SMILES: CCCCCCCCCCCC(=O)NCCCCCCNC(=O)C(CCCCNC(=O)OC(C)(C)C)NC(=O)C(CCCCNC(=O)OC(C)(C)C)NC(=O)C(N)CCCCNC(=O)OC(C)(C)C]

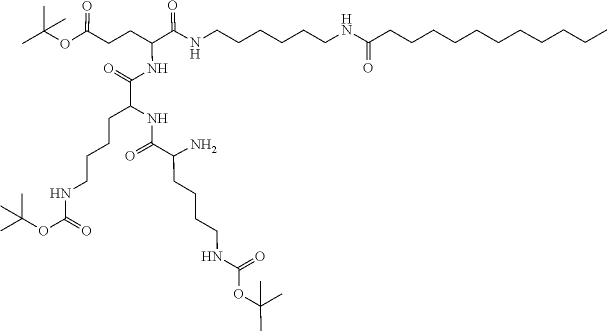 [SMILES: CCCCCCCCCCCC(=O)NCCCCCCNC(=O)C(CCC(=O)OC(C)(C)C)NC(=O)C(CCCCNC(=O)OC(C)(C)C)NC(=O)C(N)CCCCNC(=O)OC(C)(C)C]

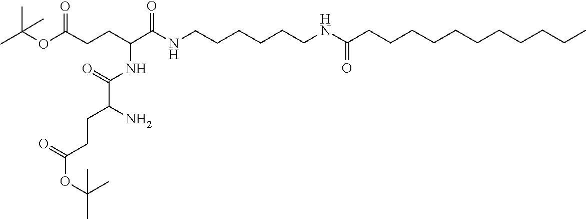 [SMILES: CCCCCCCCCCCC(=O)NCCCCCCNC(=O)C(CCC(=O)OC(C)(C)C)NC(=O)C(N)CCC(=O)OC(C)(C)C]

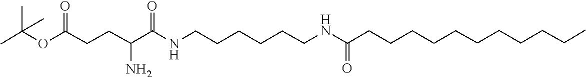 [SMILES: CCCCCCCCCCCC(=O)NCCCCCCNC(=O)C(N)CCC(=O)OC(C)(C)C]

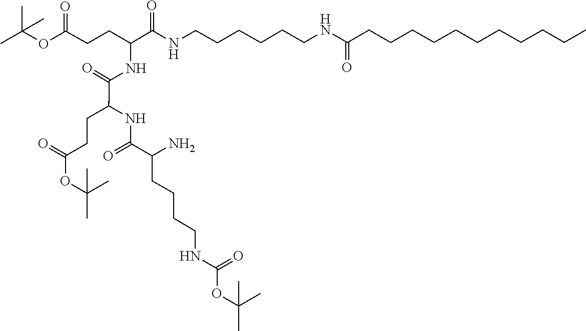 [SMILES: CCCCCCCCCCCC(=O)NCCCCCCNC(=O)C(CCC(=O)OC(C)(C)C)NC(=O)C(CCC(=O)OC(C)(C)C)NC(=O)C(N)CCCCNC(=O)OC(C)(C)C]

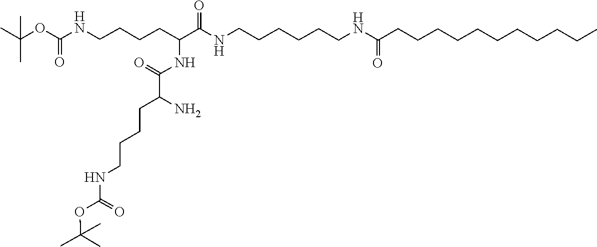 [SMILES: CCCCCCCCCCCC(=O)NCCCCCCNC(=O)C(CCCCNC(=O)OC(C)(C)C)NC(=O)C(N)CCCCNC(=O)OC(C)(C)C]

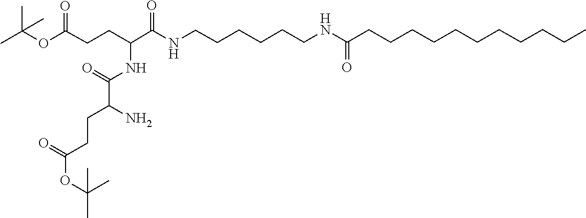 [SMILES: CCCCCCCCCCCC(=O)NCCCCCCNC(=O)C(CCC(=O)OC(C)(C)C)NC(=O)C(N)CCC(=O)OC(C)(C)C]

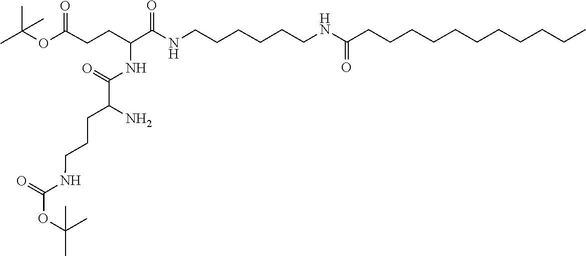 [SMILES: CCCCCCCCCCCC(=O)NCCCCCCNC(=O)C(CCC(=O)OC(C)(C)C)NC(=O)C(N)CCCNC(=O)OC(C)(C)C]

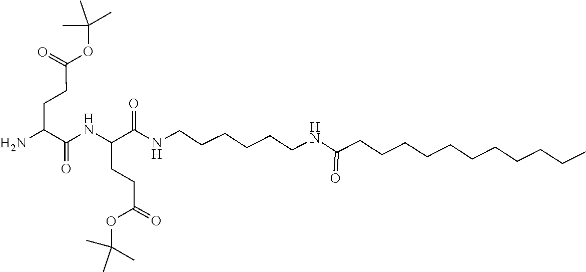 [SMILES: CCCCCCCCCCCC(=O)NCCCCCCNC(=O)C(CCC(=O)OC(C)(C)C)NC(=O)C(N)CCC(=O)OC(C)(C)C]

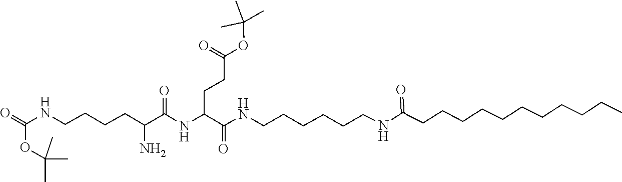 [SMILES: CCCCCCCCCCCC(=O)NCCCCCCNC(=O)C(CCC(=O)OC(C)(C)C)NC(=O)C(N)CCCCNC(=O)OC(C)(C)C]

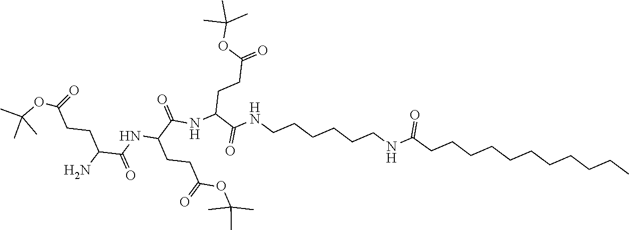 [SMILES: CCCCCCCCCCCC(=O)NCCCCCCNC(=O)C(CCC(=O)OC(C)(C)C)NC(=O)C(CCC(=O)OC(C)(C)C)NC(=O)C(N)CCC(=O)OC(C)(C)C]

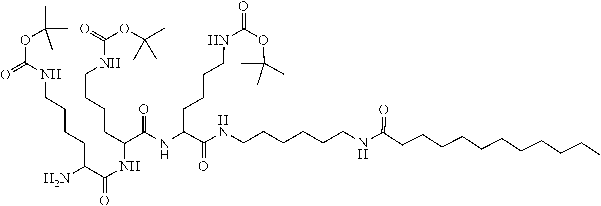 [SMILES: CCCCCCCCCCCC(=O)NCCCCCCNC(=O)C(CCCCNC(=O)OC(C)(C)C)NC(=O)C(CCCCNC(=O)OC(C)(C)C)NC(=O)C(N)CCCCNC(=O)OC(C)(C)C]

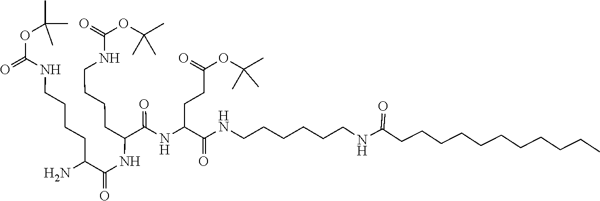 [SMILES: CCCCCCCCCCCC(=O)NCCCCCCNC(=O)C(CCC(=O)OC(C)(C)C)NC(=O)C(CCCCNC(=O)OC(C)(C)C)NC(=O)C(N)CCCCNC(=O)OC(C)(C)C]

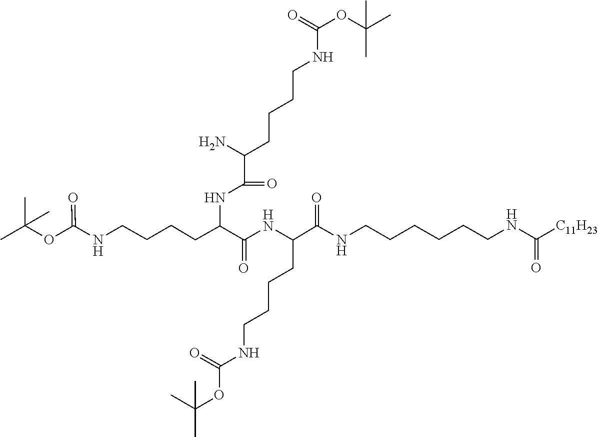

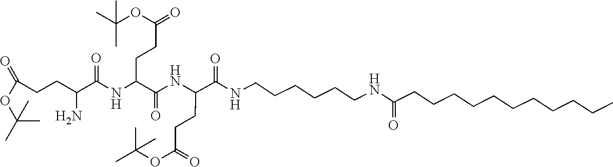 [SMILES: CCCCCCCCCCCC(=O)NCCCCCCNC(=O)C(CCC(=O)OC(C)(C)C)NC(=O)C(CCC(=O)OC(C)(C)C)NC(=O)C(N)CCC(=O)OC(C)(C)C]

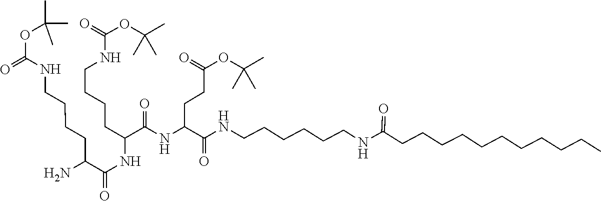 [SMILES: CCCCCCCCCCCC(=O)NCCCCCCNC(=O)C(CCC(=O)OC(C)(C)C)NC(=O)C(CCCCNC(=O)OC(C)(C)C)NC(=O)C(N)CCCCNC(=O)OC(C)(C)C]

Disclosed herein are adjuvant compositions comprising a host defense peptide, an immunostimulatory sequence and one or more cyclopolyphosphazene compounds as defined herein, such as compound 37. Embodiments of the adjuvant compositions may comprise IDR-1002 (SEQ ID NO:19) as the host defense peptide.

In some cases, the immunostimulatory sequence is poly(I:C). Embodiments of the adjuvant composition may further comprise an antigen. The antigen may be derived from a virus, bacterium, parasite, prion or fungus.

Disclosed herein are compositions comprising adjuvant compositions as described herein and a pharmaceutically acceptable excipient.

Methods of enhancing an immune response to a selected antigen are disclosed herein. The methods may comprise administering to a subject the embodiments of the adjuvant compositions, which may comprise a pharmaceutically acceptable excipient.

The adjuvant compositions are useful for the prevention and treatment of infectious diseases in humans and other animals, caused by a variety of pathogens that invade the mucosa and other parts of the body, including diseases caused by bacteria, mycobacteria, viruses, fungi, prions, parasites and the like, when used with a co-administered antigen.

### Host Defense Peptides

As explained above, the methods and compositions of the present invention include host defense peptides. Over 400 of these anti-microbial proteins have been identified in plants, insects and animals. See. e.g., Boman, H. G., Annu. Rev. Immunol. (1995) 13:61-92; Boman, H. G., Scand. J. Immunol. (1998) 48:15-25; Broekaert et al., Plant. Physiol. (1995) 108:1353-1358; Steiner et al., Nature (1981) 292:246-248; Ganz et al., Curr. Opin. Immunol. (1994) 6:584-589; Lehrer et al., Curr. Opin. Immunol. (1999) 11:23-27. The two major families of mammalian host defense peptides are defensins and cathelcidins. See. e.g., Ganz et al., Curr. Opin. Immunol. (1994) 6:584-589; Lehrer et al., supra; Ouellette et al., FASEB J. (1996) 10:1280-1289; Zanetti et al., FEBS Lett. (1995) 374:1-5.

Mammalian defensins are a family of cationic proteins that contain six highly conserved cysteine residues that form three pairs of intrachain-disulfide bonds. Mammalian defensins are classified into three subfamilies, α-, β-, and θ-defensins, based on the patterns of their intrachain-disulfide bridges, (Ganz et al., Curr. Opin. Immunol. (1994) 6:584-589; Lehrer et al., supra; Tang et al., Science (1999) 286:498-502). The θ-defensin subfamily includes a cyclic molecule with its six cysteine residues linking C₁ to C₆, C₂ to C₅, and C₃ to C₄ (Tang et al., supra). The three disulfide bonds of α-defensins are paired C₁ to C₆, C₂ to C₄, and C₃ to C₅ (Ganz et al., Curr. Opin. Immunol. (1994) 6:584-589; Ouellette et al., supra; Zhang et al., Biochemistry (1992) 31:11348-11356). The disulfide bonds of β-defensins are C1 to C5, C2 to C4, and C3 to C6 (Ganz et al., Curr. Opin. Immunol. (1994) 6:584-589; Tang et al., supra).

More than 50 defensin family members have been identified in mammalian species. In humans, at least six α-defensins and three β-defensins have been identified (Ganz et al., Curr. Opin. Immunol. (1994) 6:584-589; Lehrer et al., supra; Ouellette et al., supra; Ganz et al., J. Clin. Invest. (1985) 76:1427-1435; Wilde et al., J. Biol. Chem. (1989) 264:11200-11203; Mallow et al., J. Biol. Chem. (1996) 271:4038-4045; Bensch et al., FEBS Lett. (1995) 368:331-335; Larrick et al., Infect. Immun. (1995) 0:1291-1297). Non-limiting examples of human defensins include human α-defensins 1, 2, 3, and 4, also termed human neutrophil peptides (HNP) 1, 2, 3, and 4; human α-defensins 5 and 6 (HD5 and 6); and human β-defensins (HBD) 1, 2 and 3.

Cathelicidins are a family of anti-microbial proteins with a putative N-terminal signal peptide, a highly conserved cathelin (cathepsin L inhibitor)-like domain in the middle, and a less-conserved, C-terminal, anti-microbial domain (Lehrer et al., Curr. Opin. Immunol. (1999) 11:23-27; Zanetti et al., FEBS Lett. (1995) 374:1-5). About 20 cathelicidin members have been identified in mammals, with at least one cathelicidin from humans (Zanetti et al., supra; Larrick et al., supra; Cowland et al., FEBS Lett. (1995) 368:173-176; Agerberth et al., Proc. Natl. Acad. Sci. USA (1995) 92:195-199). Cleavage of human cathelicidin (hCAP18) liberates its C-terminal, anti-microbial domain, a peptide called LL-37, with two N-terminal leucine residues. LL-37 is 37 amino-acid residues in length (Zanetti et al., supra; Gudmundsson et al., Eur. J. Biochem. (1996) 238:325-332).

Another group of host defense peptides contains a high percentage of specific amino acids, such as the proline-/arginine-rich bovine peptides, Bac2a, Bac5 and Bac7 (Gennaro et al., Infect. Immun. (1989) 57:3142-3146) and the porcine peptide PR-39 (Agerberth et al., Eur. J. Biochem. (1991) 202:849-854); and indolicidin which is a 13-amino acid host defense peptide with the sequence ILPWKWPWWPWRR (SEQ ID NO:1).

Other representative host defense peptides are presented in Table 1 and in the examples, such as peptide IDR-1002.

The host defense peptides for use herein can include a prepro sequence, a pro-protein without the pre sequence, or the mature protein without the prepro sequence. If a signal sequence is present the molecules can include, for example, the native signal sequence, along with a pro-sequence or the mature sequence. Alternatively, a host defense peptide for use herein can include a pro sequence or mature sequence with a heterologous signal sequence. Alternatively, host defense peptide for use herein can include only the sequence of the mature protein, so long as the molecule retains biological activity. Moreover, host defense peptides for use herein can be biologically active molecules that display substantial homology to the parent molecule, as defined above.

Thus, host defense peptides for use with the present invention can include, for example, the entire parent molecule, or biologically active fragments thereof, such as fragments including contiguous amino acid sequences comprising at least about 5-10 up to about 50 to the full-length of the molecule in question, or any integer there between. The molecule will typically include one or more epitopes. Such epitopes are readily identifiable using techniques well known in the art, such as using standard antigenicity and hydropathy plots, for example those calculated using, e.g., the Omiga version 1.0 software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., supra for hydropathy plots. This program can be used with the following parameters: averaging results over a window of 7; determining surface probability according to Emini; chain flexibility according to Karplus-Schulz; antigenicity index according to Jameson-Wolf; secondary structure according to Gamier-Osguthorpe-Robson; secondary structure according to Chou-Fasman; and identifying predicted glycosylation sites. One of skill in the art can readily use the information obtained in combination with teachings of the present specification to identify antigenic regions which should be included in the molecules for use with the present invention.

Any of the above peptides, as well as fragments and analogs thereof, that display the appropriate biological activity, such as the ability to modulate an immune response, such as to enhance an immune response to a co-delivered antigen when delivered via a carrier system that also contains the other components of the adjuvant as described herein, will find use in the present methods. Enhanced adjuvant activity displayed by delivery using a carrier system can be elucidated by determining whether the composition of interest delivered with the carrier system and when co-delivered with the antigen of interest, possesses a greater capacity to elicit an immune response than the immune response elicited by an equivalent amount of the same composition delivered without a carrier system. Such enhanced immunogenicity can be determined by comparing antibody titers or cellular immune response produced using standard assays such as radioimmunoassay, ELISAs, lymphoproliferation assays, and the like, well known in the art.

The host defense peptides for use with the present invention can be obtained using standard techniques. For example, since the host defense peptides are typically small, they can be conveniently synthesized chemically, by any of several techniques that are known to those skilled in the peptide art. In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, IL 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis, all of the above are incorporated herein by reference in their entirety.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobomyloxycarbonyl, o-bromobenzyloxycarbonyl, cyclohexyl, isopropyl, acetyl, o-nitrophenylsulfonyl and the like. Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

The host defense peptides of the present invention can also be chemically prepared by other methods such as by the method of simultaneous multiple peptide synthesis. See. e.g., Houghten Proc. Natl. Acad. Sci. USA (1985) 82:5131-5135; U.S. Pat. No. 4,631,211.

Alternatively, the host defense peptides can be produced by recombinant techniques. See, e.g., Zhang et al., FEBS Lett. (1998) 424:37-40; Zhang et al., J. Biol. Chem. (1999) 274:24031-24037; Shi et al., Infect. Immun. (1999) 67:3121-3127. The host defense peptides can be produced recombinantly, e.g., by obtaining a DNA molecule from a cDNA library or vector including the same, or from host tissue using phenol extraction. Alternatively, DNA encoding the desired host defense peptide can be synthesized, along with an ATG initiation codon. The nucleotide sequence can be designed with the appropriate codons for the particular amino acid sequence desired. In general, one selects preferred codons for the intended host in which the sequence is expressed. The complete sequence is generally assembled from overlapping oligonucleotides prepared by standard methods and assembled into a complete coding sequence. See, e.g., Edge et al. Nature (1981) 292:756; Nambiar et al. Science (1984) 223:1299; Jay et al. J. Biol. Chem. (1984) 259:6311. Automated synthetic techniques such as phosphoramide solid-phase synthesis, can be used to generate the nucleotide sequence. See, e.g., Beaucage, S. L. et al. Tet. Lett. (1981) 22:1859-1862; Matteucci, M. D. et al. J. Am. Chem. Soc. (1981) 103:3185-3191. Next the DNA is cloned into an appropriate vector, either prokaryotic or eukaryotic, using conventional methods. Numerous cloning vectors are known to those of skill in the art, and the selection of an appropriate cloning vector is a matter of choice. Suitable vectors include, but are not limited to, plasmids, phages, transposons, cosmids, chromosomes or viruses that are capable of replication when associated with the proper control elements. The coding sequence is then placed under the control of suitable control elements, depending on the system to be used for expression. Thus, the coding sequence can be placed under the control of a promoter, ribosome binding site (for bacterial expression) and, optionally, an operator, so that the DNA sequence of interest is transcribed into RNA by a suitable transformant. The coding sequence may or may not contain a signal peptide or leader sequence that can later be removed by the host in post-translational processing. See. e.g., U.S. Pat. Nos. 4,431,739; 4,425,437; 4,338,397. If present, the signal sequence can be the native leader found in association with the peptide of interest.

In addition to control sequences, it may be desirable to add regulatory sequences that allow for regulation of the expression of the sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector. For example, enhancer elements may be used herein to increase expression levels of the constructs. Examples include the SV40 early gene enhancer (Dijkema et al. (1985) EMBO J. 4:761), the enhancer/promoter derived from the long terminal repeat (LTR) of the Rous Sarcoma Virus (Gorman et al. (1982) Proc. Natl. Acad. Sci. USA 79:6777) and elements derived from human CMV (Boshart et al. (1985) Cell 41:521), such as elements included in the CMV intron A sequence (U.S. Pat. No. 5,688,688). The expression cassette may further include an origin of replication for autonomous replication in a suitable host cell, one or more selectable markers, one or more restriction sites, a potential for high copy number and a strong promoter.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the sequences encoding the molecule of interest may be desirable to achieve this end. For example, in some cases it may be necessary to modify the sequence so that it can be attached to the control sequences in the appropriate orientation; i.e., to maintain the reading frame. The control sequences and other regulatory sequences may be ligated to the coding sequence prior to insertion into a vector. Alternatively, the coding sequence can be cloned directly into an expression vector that already contains the control sequences and an appropriate restriction site.

As explained above, it may also be desirable to produce mutants or analogs of the peptides of interest. Mutants or analogs of host defense peptides for use in the subject compositions may be prepared by the deletion of a portion of the sequence encoding the molecule of interest, by insertion of a sequence, and/or by substitution of one or more nucleotides within the sequence. Techniques for modifying nucleotide sequences, such as site-directed mutagenesis, and the like, are well known to those skilled in the art. See. e.g., Sambrook et al., supra; Kunkel, T. A. (1985) Proc. Natl. Acad. Sci. USA (1985) 82:448; Geisselsoder et al. (1987) BioTechniques 5:786; Zoller and Smith (1983) Methods Enzymol. 100:468; Dalbie-McFarland et al. (1982) Proc. Natl. Acad. Sci USA 79:6409.

The molecules can be expressed in a wide variety of systems, including insect, mammalian, bacterial, viral and yeast expression systems, all well known in the art. For example, insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego CA (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human embryonic kidney cells, human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti. Autographa californica. Bombyx mori. Drosophila melanogaster. Spodoptera frugiperda, and Trichoplusia ni.

Nucleic acid molecules comprising nucleotide sequences of interest can be stably integrated into a host cell genome or maintained on a stable episomal element in a suitable host cell using various gene delivery techniques well known in the art. See. e.g., U.S. Pat. No. 5,399,346.

Depending on the expression system and host selected, the molecules are produced by growing host cells transformed by an expression vector described above under conditions whereby the protein is expressed. The expressed protein is then isolated from the host cells and purified. If the expression system secretes the protein into growth media, the product can be purified directly from the media. If it is not secreted, it can be isolated from cell lysates. The selection of the appropriate growth conditions and recovery methods are within the skill of the art.

The host defense peptides, whether produced recombinantly or synthetically, are formulated into compositions and used in methods as detailed herein. Typical amounts of host defense peptides to be administered in the adjuvant compositions are from about 0.01 to about 8000 μg/kg, typically from about 0.05 to about 500 μg/kg, such as from 1 to 100 μg/kg, or 5 to 50 μg/kg, or any integer between these values.

### Immunostimulatory Sequences

Bacterial DNA is known to stimulate mammalian immune responses. See. e.g., Krieg et al., Nature (1995) 374:546-549. This immunostimulatory ability has been attributed to the high frequency of immunostimulatory nucleic acid molecules (ISSs), such as unmethylated CpG dinucleotides present in bacterial DNA. Oligonucleotides containing unmethylated CpG motifs have been shown to induce activation of B cells, NK cells and antigen-presenting cells (APCs), such as monocytes and macrophages. See. e.g., U.S. Pat. No. 6,207,646, incorporated herein by reference in its entirety.

The present invention makes use of adjuvants that include components derived from ISSs. The ISS includes an oligonucleotide that can be part of a larger nucleotide construct such as plasmid or bacterial DNA. The oligonucleotide can be linearly or circularly configured, or can contain both linear and circular segments. The oligonucleotide may include modifications such as, but are not limited to, modifications of the 3′OH or 5′OH group, modifications of the nucleotide base, modifications of the sugar component, and modifications of the phosphate group. The ISS can comprise ribonucleotides (containing ribose as the only or principal sugar component), or deoxyribonucleotides (containing deoxyribose as the principal sugar component). Modified sugars or sugar analogs may also be incorporated in the oligonucleotide. Examples of sugar moieties that can be used include ribose, deoxyribose, pentose, deoxypentose, hexose, deoxyhexose, glucose, arabinose, xylose, lyxose, and a sugar analog cyclopentyl group. The sugar may be in pyranosyl or in a furanosyl form. A phosphorous derivative (or modified phosphate group) can be used and can be a monophosphate, diphosphate, triphosphate, alkylphosphate, alkanephosphate, phosphorothioate, phosphorodithioate, or the like. Nucleic acid bases that are incorporated in the oligonucleotide base of the ISS can be naturally occurring purine and pyrimidine bases, namely, uracil or thymine, cytosine, inosine, adenine and guanine, as well as naturally occurring and synthetic modifications of these bases. Moreover, a large number of non-natural nucleosides comprising various heterocyclic bases and various sugar moieties (and sugar analogs) are available, and known to those of skill in the art.

Structurally, the root oligonucleotide of the ISS can be a CG-containing nucleotide sequence, which may be palindromic. The cytosine may be methylated or unmethylated. Examples of particular ISS molecules for use in the present invention include CpG, CpY and CpR molecules, and the like, known in the art.

Such ISS molecules can be derived from the CpG family of molecules, such as CpG dinucleotides and synthetic oligonucleotides which comprise CpG motifs (see, e.g., Krieg et al. Nature (1995) 374:546 and Davis et al. J. Immunol. (1998) 160:870-876), any of the various immunostimulatory CpG oligonucleotides disclosed in U.S. Pat. Nos. 6,194,388; 6,207,646; 6,214,806; 6,218,371; 6,239,116; 6,339,068, US Publication No. 20030139364; PCT Publication No. WO 01/22990; PCT Publication No.; and WO 03/015711, all of which are incorporated herein by reference in their entireties. Such CpG oligonucleotides generally comprise at least 8 up to about 100 nucleotides, preferably 8 to 40 nucleotides, more preferably 15-35 nucleotides, preferably 15-25 nucleotides, and any number of nucleotides between these values. For example, oligonucleotides comprising the consensus CpG motif, represented by the formula 5′-X₁CGX₂-3′, where X₁ and X₂ are nucleotides and C is unmethylated, will find use as immunostimulatory CpG molecules. Generally, X₁ is A, G or T, and X₂ is C or T. Other useful CpG molecules include those captured by the formula 5′-X₁X₂CGX₃X₄, where X₁ and X₂ are a sequence such as GpT, GpG, GpA, ApA, ApT, ApG, CpT, CpA, CpG, TpA, TpT or TpG, and X₃ and X₄ are TpT, CpT, ApT, ApG, CpG, TpC, ApC, CpC, TpA, ApA, GpT, CpA, or TpG, wherein “p” signifies a phosphate bond. Typically, the oligonucleotides do not include a GCG sequence at or near the 5′- and/or 3′ terminus. Additionally, the CpG is usually flanked on its 5′-end with two purines (preferably a GpA dinucleotide) or with a purine and a pyrimidine (preferably, GpT), and flanked on its 3′-end with two pyrimidines, such as a TpT or TpC dinucleotide. Thus, molecules can comprise the sequence GACGTT, GACGTC, GTCGTT or GTCGCT, and these sequences can be flanked by several additional nucleotides, such as with 1-20 or more nucleotides, preferably 2 to 10 nucleotides and more preferably, 3 to 5 nucleotides, or any integer between these stated ranges. The nucleotides outside of the central core area appear to be extremely amendable to change.

Moreover, the ISS oligonucleotides for use herein may be double- or single-stranded. Double-stranded molecules are more stable in vivo while single-stranded molecules display enhanced immune activity. Additionally, the phosphate backbone may be modified, such as phosphorodithioate-modified, in order to enhance the immunostimulatory activity of the ISS molecule. As described in U.S. Pat. No. 6,207,646, CpG molecules with phosphorothioate backbones preferentially activate B-cells, while those having phosphodiester backbones preferentially activate monocytic (macrophages, dendritic cells and monocytes) and NK cells.

Different classes of CpG nucleic acids have been described. One class is potent for activating B cells but is relatively weak in inducing IFN-α and NK cell activation. This class has been termed the B class. The B class CpG nucleic acids are fully stabilized and include an unmethylated CpG dinucleotide within certain preferred base contexts. See. e.g., U.S. Pat. Nos. 6,194,388; 6,207,646; 6,214,806; 6,218,371; 6,239,116; and 6,339,068, incorporated herein by reference in their entireties. Another class is potent for inducing IFN-α and NK cell activation but is relatively weak at stimulating B cells; this class has been termed the A class. The A class CpG nucleic acids typically have stabilized poly-G sequences at 5′ and 3′ ends and a palindromic phosphodiester CpG dinucleotide-containing sequence of at least 6 nucleotides. See, for example, PCT Publication No. WO 01/22990, incorporated herein by reference in its entirety. Yet another class of CpG nucleic acids activates B cells and NK cells and induces IFN-α; this class has been termed the C-class. The C-class CpG nucleic acids typically are fully stabilized, include a B class-type sequence and a GC-rich palindrome or near-palindrome. This class has been described in PCT Publication No. WO 03/015711, the entire contents of which is incorporated herein by reference.

ISS molecules can readily be tested for their ability to stimulate an immune response using standard techniques, well known in the art. For example, the ability of the molecule to stimulate a humoral and/or cellular immune response is readily determined using the immunoassays described herein. Moreover, the adjuvant compositions and antigen can be administered with and without the ISS to determine whether an immune response is enhanced.

Exemplary, non-limiting examples of CpG oligonucleotides for use in the present compositions include those oligonucleotides 5′TCCATGACGTTCCTGACGTT3′ (SEQ ID NO:8), termed CpG ODN 1826, a Class B CpG; 5′TCGTCGTTGTCGTITTGTCGTT3′ (SEQ ID NO:9), termed CpG ODN 2007, a Class B CpG; 5′TCGTCGTITTGTCGTITTGTCGTT3′ (SEQ ID NO:10), also termed CPG 7909 or 10103, a Class B CpG; 5′ GGGGACGACGTCGTGGGGGGG 3′ (SEQ ID NO:11), termed CpG 8954, a Class A CpG; and 5′TCGTCGTITTCGGCGCGCGCCG 3′ (SEQ ID NO:12), also termed CpG 2395 or CpG 10101, a Class C CpG. All of the foregoing class B and C molecules are fully phosphorothioated.

Non-CpG oligonucleotides for use in the present composition include the double stranded polyriboinosinic acid:polyribocytidylic acid, also termed poly(I:C); and a non-CpG oligonucleotide

(SEQ ID NO: 13)
5′AAAAAAGGTACCTAAATAGTATGTTTCTGAAA3′.

Generally, the ISS present in the adjuvant composition will represent about 0.01 to about 1000 μg/kg, typically from about 0.05 to about 500 μg/kg, such as from 1 to 100 μg/kg, or 5 to 50 μg/kg, or any amount within these ranges, of the ISS per dose. One of skill in the art can determine the amount of ISS, as well as the ratio of ISS to the other components in the adjuvant composition.

### Vaccine Antigens

The adjuvant compositions are able to enhance a local immune response, and in some cases systemic immunity, to a co-delivered vaccine antigen. An adjuvant composition comprising a host defense peptide, a polyphosphazene and an immunostimulatory sequence, enhances the immune response vis-a-vis a selected antigen when it possesses a greater capacity to elicit an immune response than the immune response elicited by an equivalent amount of the antigen when delivered without the adjuvant composition. Such enhanced immunogenicity can be determined by administering the antigen and the adjuvant composition, and antigen controls to animals and comparing antibody titers against the two using standard assays such as radioimmunoassay and ELISAs, well known in the art.

Antigens for use with the adjuvant compositions include, but are not limited to, antigens of viral, bacterial, mycobacterial, fungal, or parasitic origin.

For example, the adjuvant compositions of the invention can be used in combination with antigens to treat or prevent a wide variety of infections caused by bacteria, including gram-negative and gram-positive bacteria. Particularly useful antigens for stimulating mucosal immunity will be derived from pathogens that invade the mucosa, such as, but not limited to pathogens that invade the respiratory tract, the GI tract, the urogenital tract and the eye.

Non-limiting examples of bacterial pathogens from which antigens can be derived include both gram negative and gram positive bacteria. Gram positive bacteria include, but are not limited to Pasteurella species, Staphylococci species, and Streptococcus species. Gram negative bacteria include, but are not limited to, Escherichia coli, Lawsonia intracellularis, Pseudomonas species, and Salmonella species. Specific examples of infectious bacteria include but are not limited to: Helicobacter pylori, Borelia burgdorferi, Legionella pneumophilia, Mycobacteria sp. (e.g. M. tuberculosis, M. avium, M. intracellulare, M. kansaii, M gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcusfaecalis, Streptococcus bovis, Streptococcus (anaerobic sp.), Streptococcus pneumoniae, pathogenic Campylobacter spp., Enterococcus sp., Haemophilus infuenzae, Bacillus antracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

For example, the adjuvant compositions of the present invention can be used with any of the various Bordetella species including B. pertussis, B. parapertussis, B. bronhiseptica, and the like; various Neisserial species, including N. meningitidis, N. gonorrhoeae, etc.; various Enterobacteriaceae such as but not limited to Salmonella, such as S. typhimurium, S. enteritidis, Shigella, such as S. flexneri, Escherichia, such as E. coli O157:H7, Klebsiella, Enterobacter, Serratia, Proteus, Morganella, Providencia, Yersinia. such as Y. enterocolitica. Listeria, such as L. monocytogene, Staphylococcus, such as S. aureus; various Pseudomonas species, such as P. aeruginosa; Stretococcal species, such as S. suis, S. uberis, S. agalactiae, S. dysgalactiae, S. pneumoniae, S. pyogenes, and the like; various Actinobacillus species, including but not limited to A. Pleuropneumoniae, A. suis, A. pyogenes, etc.

The adjuvant compositions can be used in combination with antigens to treat or prevent diseases caused by improper food handling, as well as diseases caused by food-borne pathogens, such as but not limited to Salmonella enteritidis, Salmonella typhimurium, Escherichia coli 057:H7, Yersinia enterocolitica, Shigella flexneri, Listeria monocytogene, and Staphylococcus aureus. Additionally, the adjuvant compositions are also useful in combination with antigens from pathogens that cause nosocomial infections, such as but not limited to pathogens that produce extended spectrum β-lactamases (ESBL) and thus have the ability to inactivate 0-lactam antibiotics. These enzymes are produced by various bacteria, including Klebsiella pneumoniae, E. coli and Proteus mirabilis. Additionally, the adjuvant compositions can be used in combination with antigens to treat or prevent diseases caused by biocontamination of the skin by pathogenic microorganisms such as Staphylococcus aureus, S. epidermitidis, Pseudomonas aeruginosa, Acinetobacter spp., Klebsiella pneumoniae, Enterobacter cloacae, E. coli, Proteus spp. and fungi such as Candida albicans.

The adjuvant compositions can also be used in combination with antigens to treat or prevent respiratory conditions such as caused by Streptococcus pneumoniae, Haemophilus influenzae, and Pseudomonas aeruginosa, as well as sexually transmitted diseases, including but not limited to Chlamydia infections, such as caused by Chlamydia trachomatis and gonococcal infections, such as caused by Neisseria gonorrhoeae.

Additionally, the adjuvant compositions can be used with antigens to treat or prevent a number of viral diseases, such as but not limited to those diseases caused by members of the families Picomaviridae (e.g., polioviruses, etc.); Caliciviridae; Togaviridae (e.g., rubella virus, dengue virus, etc.); Flaviviridae; Coronaviridae; Reoviridae; Bimaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; See. e.g. Virology, 3rd Edition (W. K. Joklik ed. 1988); Fundamental Virology, 2nd Edition (B. N. Fields and D. M. Knipe, eds. 1991), for a description of these and other viruses. Other particular examples of viruses include the herpesvirus family of viruses, for example bovine herpes virus (BHV) and human herpes simplex virus (HSV) types 1 and 2, such as BHV-1, BHV-2, HSV-1 and HSV-2, varicella zoster virus (VZV), Epstein-Barr virus (EBV), cytomegalovirus (CMV), HHV6 and HHV7; diseases caused by the various hepatitis viruses, such as HAV, HBV and HCV; diseases caused by papilloma viruses and rotaviruses, etc.

Non-limiting examples of viral pathogens that affect humans and/or nonhuman vertebrates from which antigens can be derived, or which can be provided in attenuated or inactivated form include retroviruses, RNA viruses and DNA viruses. The group of retroviruses includes both simple retroviruses and complex retroviruses. The simple retroviruses include the subgroups of B-type retroviruses, C-type retroviruses and D-type retroviruses. An example of a B-type retrovirus is mouse mammary tumor virus (MMTV). The C-type retroviruses include subgroups C-type group A (including Rous sarcoma virus, avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses and the foamy viruses. Lentiviruses include HIV-1, HIV-2, SIV, Visna virus, feline immunodeficiency virus (FIV), and equine infectious anemia virus (EIAV). The T-cell leukemia viruses include HTLV-1, HTLV-II, simian T-cell leukemia virus (STLV), and bovine leukemia virus (BLV). The foamy viruses include human foamy virus (HFV), simian foamy virus (SFV) and bovine foamy virus (BFV).

Examples of other RNA viruses from which antigens can be derived include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picomaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picomavirus and Norwalk virus; the family Togaviridae, including the genus Alphavirus (Eastern equine encephalitis virus, Semliki forest virus, Sindbis virus, Chikungunya virus, ONyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick bome virus, Kyasanur forest virus, Louping III virus, Powassan virus, Omsk hemorrhagic fever virus), the genus Rubivirus (Rubella virus), the genus Pestivirus (Mucosal disease virus, BVDV, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus (BRSV), and Pneumonia virus of mice); forest virus, the family Rhabdoviridae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including the SARS virus, Infectious Bronchitis Virus (IBV), Mouse Hepatitis virus, Human enteric corona virus, Porcine epidemic diarrhea virus (PEDV) and Feline infectious peritonitis (Feline coronavirus). For example, for RSV vaccines, useful antigens include those derived from the fusion (F) protein, the attachment (G) protein, and/or the matrix (M) protein, or combinations thereof. These proteins are well known and can be obtained as described in U.S. Pat. No. 7,169,395, incorporated herein by reference in its entirety.

Illustrative DNA viruses from which antigens can be derived include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex virus Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, porcine parvovirus, Aleutian mink disease virus, etc). Finally, DNA viruses may include viruses which do not fit into the above families such as Kuru and Creutzfeldt-Jacob disease viruses and chronic infectious neuropathic agents (CHINA virus).

Additionally, the adjuvant compositions can be used with antigens to treat or prevent a number of prion diseases, such as but not limited to those diseases known in the art, such as Creutzfeldt-Jacob disease, transmissible spongiform encephalopathies, bovine spongiform encephalopathies, scrapie, and others. See. e.g. N A Mabbott, Prospects for safe and effective vaccines against prion diseases, Expert Rev Vaccines. 2015 January; 14(1):1-4, herein incorporated by reference.

Similarly, the adjuvant compositions of the invention will find use against a variety of parasites, such as but not limited to Plasmodium, such as P. malariae, P. yoelii, P. falciparum, P. ovale, and P. vivax, Toxoplasma gondii, Schistosoma japonicum, Leishmania major, Trypanosoma cruzi, and so forth.

Additionally, the adjuvant compositions find use to enhance an immune response against a number of fungal pathogens, such as but not limited to those fungi causing Candidiasis, Cryptococcosis, Asperigillosis, Zygomycosis, Blastomycosis, Coccidioidomycosis, Histoplasmosis, Paracoccidiodomycosis, Sporotrichosis. Particular non-limiting examples of infectious fungi from which antigens can be derived include: Cryptococcus neoformans, Histoplasma capsulatum, Coccidioides immitis, Blastomyces dermatitidis, Chlamydia trachomatis, Candida albicans.

Other medically relevant microorganisms have been described extensively in the literature. See. e.g. C. G. A Thomas, Medical Microbiology, Bailliere Tindall, Great Britain 1983, the entire contents of which is hereby incorporated by reference.

Thus, it is readily apparent that the adjuvant compositions can be used in combination with a wide variety of antigens to enhance the immune response to prevent or treat diseases, such as infectious disease in humans, as well diseases in non-human animals.

These antigens can be provided as attenuated, inactivated or subunit vaccine compositions. Additionally, the antigens can be provided in nucleic acid constructs for DNA immunization. Techniques for preparing DNA antigens are well known in the art and described in, e.g., U.S. Pat. Nos. 5,399,346, 5,580,859, 5,589,466, incorporated by reference herein in their entireties.

The adjuvant compositions are also useful in combination with a number of commercial vaccines, in order to enhance an immune response to the co-delivered antigen. For example, the adjuvant compositions can be co-administered with commercially available human and animal vaccines, including but not limited to pertussis vaccines and combination vaccines, such as the various whole cell (wP) and acellular vaccines (aP). Nonlimiting examples of such vaccines include the vaccines known as TRIPEDIA, TRIPACEL, QUADRACEL, TETRAVAL, TETRACT-Hib, PENTACT-Hib, PENTACEL, PENTAVAC, and HEXAVAC (Aventis, Bridgewater, NJ); INFANRIX and PEDIARIX (GlaxoSmithKline, Research Triangle Park, NC); CERTIVA (North American Vaccine, Beltsville, MD); BIOTHRAX; TICE BCG; MYCOBAX; HiBTITER; PEDVAXHIB; ACTHIB; COMVAX; HAVRIX; VAQTA; TWINRIX; RECOMBIVAX HB; ENGERIX-B; FLUMIST; FLUVIDRIN; FLUZONE; JE-VAX; ATTENUVAX; M-M-VAX; M-M-R II; MENUMONE-A/C/Y/W-135; MUMPSVAX; PNEUMOVAX 23; PREVNAR; POLIOVAX; IPOL; IMOVAX; RABAVERT; MERUVAX II; DRYVAX; TYPHIM Vi; VIVOTIF; VARIVAX; YF-VAX.

The antigens for use with the present invention can be prepared using standard techniques, well known in the art. For example, the antigens can be isolated directly from the organism of interest, or can be produced recombinantly or synthetically, using techniques described above.

### Formulations and Administration

The adjuvant composition and the antigen may be formulated for delivery to a subject. In some cases, the composition is formulated for systemic administration, such as intramuscular delivery. In some cases, the composition is formulated for mucosal administration, such as to the buccal cavity, sublingually, the nasal passages, the lungs, the GI tract, the eye, the urogenital tract, and the like. Some embodiments of formulations include suppositories, aerosol, intranasal, oral formulations, and sustained release formulations. Methods of preparing such formulations are known in the art and described in, e.g., Remington's Pharmaceutical Sciences, Mack Publishing Company, Easton, Pennsylvania, Current edition.

Embodiments of the adjuvant compositions may be formulated for intramuscular delivery. Methods of preparing such formulations are known in the art and described in, e.g., Remington's Pharmaceutical Sciences, Mack Publishing Company, Easton, Pennsylvania, Current edition.

Intranasal formulations may include pharmaceutically acceptable excipients that neither cause major irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa. Agents can be delivered intranasally using nasal drops, sprays, gels, suspensions and emulsions, an inhaler and/or an atomizer. Thus, the intranasal formulation may be administered by methods such as inhalation, spraying, liquid stream lavage, nebulizing, or nasal irrigation. The administering may be to the sinus cavity or the lungs.

For suppositories, the excipients will include traditional binders and carriers, such as, polyalkaline glycols, or triglycerides. Such suppositories may be formed from mixtures containing the active ingredient in the range of about 0.5% to about 10% (w/w), preferably about 1% to about 2%.

Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Aerosol delivery systems may employ nebulizers and other inhaler devices and systems. Delivering drugs by inhalation requires a formulation that can be successfully aerosolized and a delivery system that produces a useful aerosol of the drug. The particles or droplets should be of sufficient size and mass to be carried to the distal lung or deposited on proximal airways to give rise to a therapeutic effect.

Vaccination is achieved in a single dose or repeated as necessary at intervals, as can be determined readily by one skilled in the art. For example, a priming dose can be followed by one or more booster doses at weekly, monthly, or longer intervals. An appropriate dose depends on various parameters including the recipient (e.g., adult or infant), the particular vaccine antigen, the route and frequency of administration, and the desired effect (e.g., protection and/or treatment), as can be determined by one skilled in the art. The adjuvant composition, and optionally a vaccine antigen, may be administered in an amount from 1 to 25 μg per kg.

The present invention will be further illustrated in the following examples.

### Example 1: In Vivo Studies: Vaccination in Chicken with SipD as an Antigen

To assess the adjuvant activity of cyclopolyphosphazene candidates, such as 37, 11 and 39, an in vivo study was conducted with intramuscular administration of a Salmonella typhimurium Cell invasion protein (SipD) vaccine in young chicken. Two-week-old Leghorn chicken were randomly divided into three single adjuvant groups, three triple adjuvant groups and one PBS-adjuvant control group (n=12/group). The single adjuvant groups contained 20 μg PCEP, 11 or 37 polyphosphazene. All triple adjuvants comprised Poly(I:C), IDR-1002 peptide and a polyphosphazene: either PCEP, 37 or 11. The dose of TriAdj had a constant weight ratio of polyphosphazene:peptide:Poly(I:C) of 20 μg:40 μg:20 μg.

All vaccines were formulated prior to administration and injected in 200 μL intramuscularly in the tail area. Chicken were vaccinated at Day 0 and Day 14 (Week 2) with the same dose. Treatment Groups: A: PBS control group (no antigen); B: SipD control 10 μg (antigen only, no adjuvant); C: SipD 10 μg formulated with PCEP; D: SipD 10 μg formulated with 11; E: SipD 10 μg formulated with 37; F: SipD 10 μg formulated with PCEP-TriAdj; G: SipD 10 μg formulated with 11-TriAdj; H: SipD 10 μg formulated with 37-TriAdj.

Serum was collected on days 0, 14 and 28 for SipD-antigen-specific IgG ELISAs. The analyst was blinded to treatment group during the ELISA assays. ELISAs were performed on the collected sera as follows: Plates were coated overnight with SipD at 4° C. and incubated with sera starting from 1/40 and serially diluted. To detect IgG, alkaline phosphatase-labeled goat anti-chicken IgG (H+L) was added (KPL catalogue #KP-151-24-06). A colorimetric reaction was developed using p-nitrophenyl phosphate (Sigma-Aldrich, St. Louis, MO) as the AP substrate. Plates were read with a Molecular Devices SpectraMax Plus 384 Reader™. Data were expressed as titres, which represent the dilution factor required to generate an absorbance reading two standard deviations above the average value of the negative control, e.g. serum from control chicken receiving no vaccination.

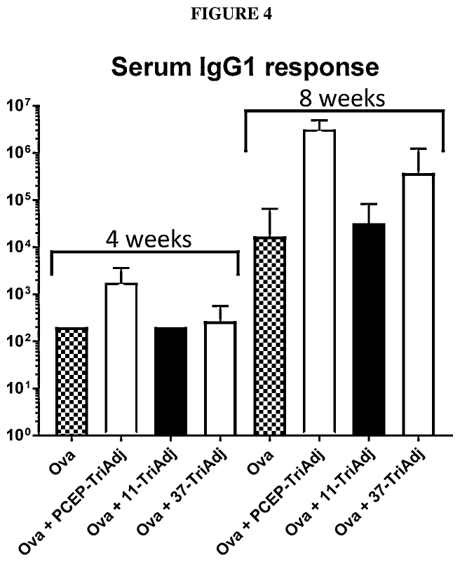

The results obtained from the studies in chicken are shown in FIG. 1; IgG titres at Week 0 before the first immunization constitute the baseline to which the titres at Weeks 2 (two weeks after the first immunization, FIG. 1A) and 4 (two weeks after the second immunization and four weeks after the first immunization, FIG. 1B) can be compared. FIG. 1B shows a significantly greater response in antigen-specific IgG titres with the triple adjuvant composition that contained 37 or PCEP relative to a vaccine without adjuvant, after two immunizations.

### Materials and Methods

Polyinosinic-Polycytidylic acid (poly(I:C)) double-stranded RNA adjuvant (99% purity) was obtained from Sigma Aldrich (Canada). IDR-1002 was obtained from Genscript (Piscataway Township, NJ). The sequence of IDR-1002 is

(SEQ ID NO: 19)
Val-Gln-Arg-Trp-Leu-Ile-Val-
Trp-Arg-Ile-Arg-Lys-NH2.

Poly(I:C) and IDR-1002 cationic peptide were used in the formulation with one polyphosphazene.

The poly(di-4-oxyphenylproprionate)phosphazene (PCEP), sodium salt, was obtained by custom synthesis at Idaho National Laboratory and can be prepared as described in U.S. Pat. Nos. 9,408,908 and 9,061,001, each of which is incorporated herein by reference in its entirety. The polyphosphazene tested endotoxin free.

General chemicals were from Sigma Aldrich (Canada). SipD was a recombinant protein expressed as described in TS Desin et al., Infection and Immunity (2009), p. 2866-2875.

### Example 2: In Vivo Studies: Vaccination in Mice with Ovalbumin as an Antigen

To assess the adjuvant activity of the cyclopolyphosphazene 37, four in vivo studies were conducted with intramuscular administration of an ovalbumin (Ova) vaccine in mice. In the first two studies, the Ova vaccine was either formulated with no adjuvant or with a triple adjuvant (TriAdj). The first two studies compared three different triple adjuvants, all comprising Poly(I:C), IDR-1002 peptide and a polyphosphazene: either PCEP, 37 or 11. The third and fourth studies compared four different triple adjuvants, all comprising Poly(I:C), IDR-1002 peptide and a polyphosphazene: either PCEP, compound 11, compound 37 or compound 39. In all studies, the dose of TriAdj had a constant weight ratio of polyphosphazene:peptide:Poly(I:C) of 10 μg:20 μg:10 μg.

In all studies, mice were randomized to cages such that the various treatment groups were not together in the same cage. All groups received Ova antigen mixed with the adjuvant (or not) just prior to intramuscular administration. All the vaccines were formulated and injected in 50 μL (25 μ/leg). Mice were vaccinated at Day 0 and Day 28 (Week 4) with the same dose.

In all studies, serum was collected on days 0, 28 and 56 for antigen-specific IgG1 and IgG2a ELISAs. To detect IgG1 and IgG2a, biotin-labeled goat anti-mouse IgG1 or IgG2a was added (IgG1: Invitrogen Cat. No. A10519; IgG2a: Invitrogen Cat. No. M32315) followed by streptavidin-alkaline phosphatase (AP) (016-050-084, Jackson ImmunoResearch Laboratories Inc., West Grove, PA). A colorimetric reaction was developed using p-nitrophenyl phosphate (Sigma-Aldrich, St. Louis, MO) as the AP substrate. Plates were read with a Biorad iMark Microplate Reader™. Data were expressed as titres, which represent the dilution factor required to generate an absorbance reading three standard deviations above the average value of the negative control, e.g. serum from control mice receiving no vaccination.

In a first study, female Balb/c mice were randomly divided into three adjuvant groups and one PBS control group (n=6/group). Treatment Groups: A: Ova control 50 μg (antigen only, no adjuvant); B: Ova 50 μg formulated with PCEP-TriAdj; C: Ova 50 μg formulated with 11-TriAdj; D: Ova 50 μg formulated with 37-TriAdj.

In a second study, female Balb/c mice were randomly divided into three adjuvant groups and one PBS control group (n=8/group). Treatment Groups: A: Ova control 1 μg (antigen only, no adjuvant); B: Ova 1 μg formulated with PCEP-TriAdj; C: Ova 1 μg formulated with 11-TriAdj; D: Ova 1 μg formulated with 37-TriAdj.

In a third study, female Balb/c mice were randomly divided into four adjuvant groups (n=8/group). Treatment Groups: A: Ova control 1 μg formulated with 11-TriAdj; B: Ova 1 μg formulated with 39-TriAdj; C: Ova 1 μg formulated with 37-TriAdj; D: Ova 1 μg formulated with PCEP-TriAdj.

In a fourth study, female Balb/c mice were randomly divided into four adjuvant groups and one PBS control group (n=8/group). Treatment Groups: A: Ova control 1 μg (antigen only, no adjuvant); B: Ova 1 μg formulated with 11-TriAdj; C: Ova 1 μg formulated with 37-TriAdj; D: Ova 1 μg formulated with 39-TriAdj; E: Ova 1 μg formulated with PCEP-TriAdj.

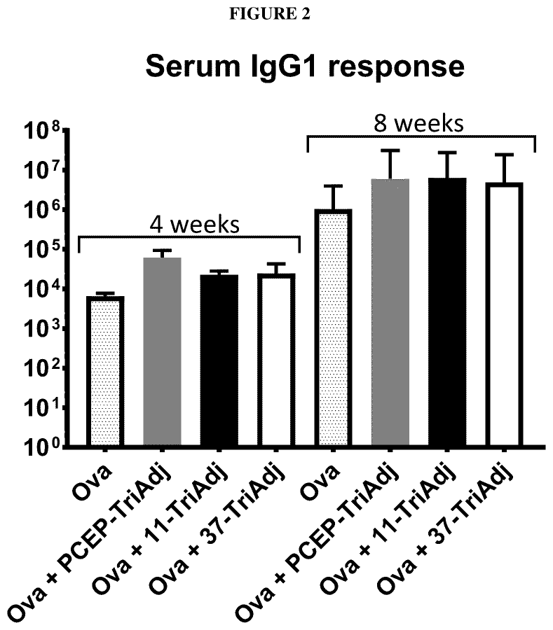

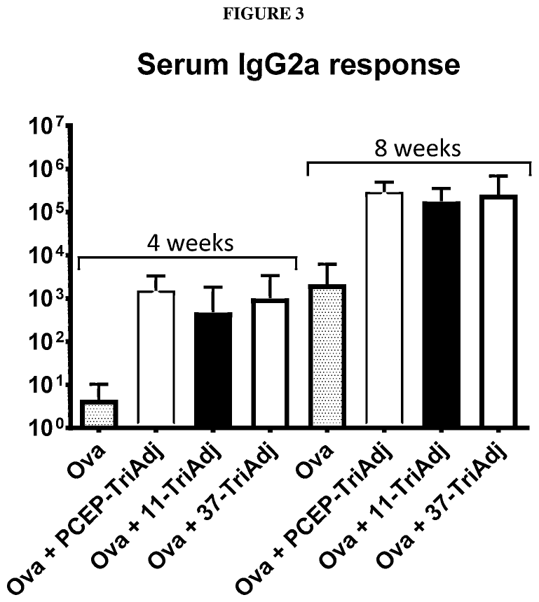

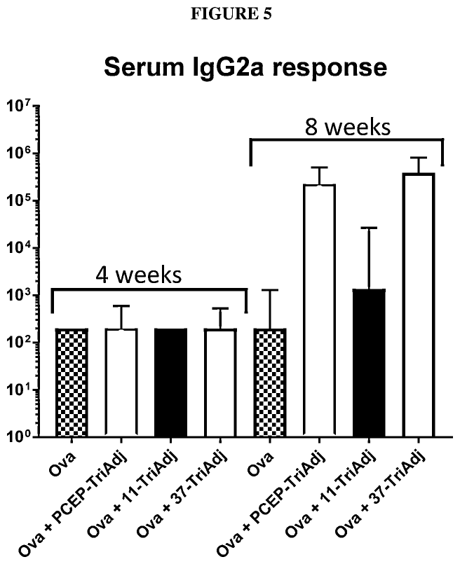

The results obtained from the studies in mice are shown in FIGS. 2-9; titers at Week 0 before the first immunization constitute the baseline to which the titers at Weeks 4 (four weeks after the first immunization) and 8 (four weeks after the second immunization) can be compared. FIGS. 2 and 5 show a significantly greater response in IgG1 after two immunizations (Week 8) with the triple adjuvant composition that contained 37 or PCEP relative to a vaccine without adjuvant. FIGS. 3 and 5 show a significantly greater response in IgG2a after two immunizations (Week 8) with the triple adjuvant composition that contained 37 or PCEP relative to a vaccine without adjuvant.

Polyinosinic-Polycytidylic acid (poly(I:C)) double-stranded RNA adjuvant (99% purity) was obtained from Sigma Aldrich (Canada). IDR-1002 was obtained from Genscript (Piscataway Township, NJ). The sequence of IDR-1002 is Val-Gln-Arg-Trp-Leu-Ile-Val-Trp-Arg-Ile-Arg-Lys-NH2 (SEQ ID NO:19).

Poly(I:C) and IDR-1002 cationic peptide were used in the formulation with one polyphosphazene.

The poly(di-4-oxyphenylproprionate)phosphazene (PCEP), sodium salt (average molecular weight approximately 1800×10³), was obtained by custom synthesis at Idaho National Laboratory and can be prepared as described in U.S. Pat. Nos. 9,408,908 and 9,061,001, each of which is incorporated herein by reference in its entirety. The polyphosphazene tested endotoxin free.

General chemicals, including ovalbumin from chicken egg white (Ova), were from Sigma Aldrich (Canada).

### Example 3: Synthesis of Cyclopolyphosphazenes

DMF, dimethylformamide; DCM, dichloromethane; ACN, acetonitrile; DMSO, dimethylsulfoxide; HDA, 1,6-diaminohexane linker; C12. Dodecanoyl aliphatic chain; Lys, L-lysine; Glu, L-glutamic acid; tfa, trifluoroaceic acid; FMoc, fluorenylmethoxy carbonyl; t-Boc, tertiary butyloxycarbonyl; hplc, high performance liquid chromatography. The MALDI-ToF analyses are run on the 4800 MALDI TOF/TOF Analyzer mass spectrometer from Applied Biosystems Life Sciences.

### General Procedure for Synthesis of Cyclopolyphosphazenes (FIG. 10)

The following procedure was used to synthesize an illustrative embodiment and a person of skill in the art will understand that the synthesis can be altered at the appropriate steps to obtain the claimed embodiments.

### Synthesis of compound 5—Hexamethyl 3,3′,3″,3′″,3″″,3′″″-(((1,3,5,2l5,4l5,6l5-triazatriphosphinine-2,2,4,4,6,6-hexayl)hexakis(oxy))hexakis(benzene-4,1-diyl))hexapropionate

Potassium carbonate (VWR International) was ground to a powder and dried in an oven at 140° C. for 1 hr. Solvents (VWR international) Hexachlorotriphosphazene, methyl-3-(4-hydroxyphenyl)propionate, tetra-n-butylammonium bromide (Sigma-Aldrich) were used without further purification

A mixture of hexachlorotriphosphazene, compound 3 (1.6 g, 4.6 mmol), methyl-3-(4-hydroxyphenyl)propionate, compound 4 (5 g, 27.7 mmol), potassium carbonate (13 g, 65.78 mmol) and tetra-n-butylammonium bromide (2.13 g, 6.6 mmol) in acetone, 150 mL was stirred at room temperature for 1 h and heated to reflux with stirring for 12 hr. A further tetra-n-butyl ammonium bromide (1 g, 3.0 mmol) was added and refluxed for 8 hr.

The reaction mixture was allowed to cool to room temperature and the acetone layer decanted. The residue is washed with acetone and filtered. The acetone solutions were combined. The solvent was evaporated on a rotary evaporator and the crude product isolated. The isolated product was crystallized from ethanol to yield compound 5 (4.1 g, 77.3% yield).

¹H-NMR: δ (CDCl₃) 7.0 (d, J=8.4), 6.83 (d, J=8.4), 3.65, (s), 2.89 (t, J=7.7), 2.59 (t, J=8.0). ³¹P-NMR: δ (Acetone) 8.88 (s). MALDI ToF mass spectrum. 1210.63; Calc'd: C₆₀H₆₆N₃O₁₈P₃: 1209.36.

### Synthesis of compound 6-3,3′,3″,3′″,3″″,3′″″-(((1,3,5,2l5,4l5,6l5-triazatriphosphinine-2,2,4,4,6,6-hexayl)hexakis(oxy))hexakis(benzene-4,1-diyl))hexapropionic acid

Compound 5 (2.5 g, 2.06 mmol) was dissolved in acetone, 60 ml and cooled on ice. Sodium hydroxide (28 ml of 3.0 M solution, 84 mmol) was added and the ice bath removed and the solution stirred for 2 hr cooled again and acidified with hydrochloric acid (10 ml conc. Hydrochloric acid: 10 ml water). The acetone was partially evaporated under reduced pressure and water added to the resulting solution to precipitate the product. The crude product was filtered, washed with, water and ethyl acetate. The product was recrystallized from ethanol to give compound 6 (2.15 g, 97% yield)

¹H-NMR: δ (D₆acetone, 10% DMSO-d₆) 7.17 (d, J=8.5), 6.83 (d, J=8.5), 2.9 (t, J=7.6), 2.59 (t, J=7.6). ³¹P-NMR: δ (D₆acetone, DMSO-d₆) 9.16. MALDI ToF mass spectrum. 1126.36; Calc'd: C₅₄H₅₄N₃O₁₈P₃: 1125.26.

### Synthesis of compound 7—Hexakis(2,5-dioxopyrrolidin-1-yl) 3,3′,3″,3′″,3″″,3′″″-(((1,3,5,215,415,615-triazatriphosphinine-2,2,4,4,6,6-hexayl)hexakis(oxy))hexakis(benzene-4,1-diyl))hexapropionate

Hexakis-(4-oxyphenylpropionic)cyclotriphosphazene acid, (compound 6, 1.41 g, 1.25 mmol) and N-hydroxysuccinimide (1.0 g, 8.66 mmol) was dissolved in dichloromethane, 12 ml and dimethylformamide, 4 ml. N,N′-diisopropylcarbodimimide (1.23 g, 9.7 mmol), 1.53 ml, was added and stirred at room temperature for 18 hr. The insoluble precipitate was filtered off and discarded. Diethyl ether was added to the filtrate and the solvent decanted from the settled ‘gummy-oily’ residue. The residue was washed with diethyl ether. The final residue was dissolved in warm ethyl acetate, filtered to remove insoluble material and the solvent evaporated from the filtrate. The residue was dried under vacuum to give a powdery crude product compound 7. (1.86 g, 88.5% yield). This product was used without further purification.

¹H-NMR: δ (CDCl₃), 10% (DMSO-d₆) 7.08 (d, J=8.4), 6.89 (d, J=8.4), 3.03 (t, J=7.4), 2.086-2.98 (m). MALDI ToF mass spectrum. 1708.52, 1730.47 (M+Na⁺); Calc'd:C₇₈H₇₂N₉O₃₀P₃: 1707.36

### Synthesis of compound 37—N,N′,N″,N′″,N″″,N′″″-(((2,2′,2″,22′″,2″″,2′″″-((3,3′,3″,3′″,3″″,3′″″-(((1,3,5,2l5,4l5,6l5-triazatriphosphinine-2,2,4,4,6,6-hexayl)hexakis(oxy))hexakis(benzene-4,1-diyl))hexakis(propanoyl))hexakis(azanediyl))hexakis(6-aminohexanoyl))hexakis(azanediyl))hexakis(hexane-6,1-diyl))hexadodecanamide

To compound 7 (80.0 mg, 46.8 μmol) dissolved in chloroform (2 mL) was added compound 19 (150.39 mg, 0.285 mmol) dissolved in chloroform (3 mL), and stirred at room temperature for 20 hr. The solvent was evaporated under vacuum. Trifluoroacetic acid (3 mL) was added to the residue and stirred for 2 hr. The trifluoroacetic acid was evaporated under vacuum and the residue dissolved in warm acetonitrile. Diethyl ether was added to the solution until the solution became cloudy.

The cloudy solution was kept at room temperature for 1 hr. The precipitated material was filtered and washed with diethyl ether to give the crude product. The crude product was suspended in dichloromethane, filtered washed with dichloromethane and dried to give compound 37 (153.7 mg, 91.78% yield)

¹H-NMR: δ (D₂O), 7.09 (d, J=8.5, 2H), 6.79 (d, J=8.4, 2H), 4.29 (dd, J=11.3, 1H), 3.16 (t, J=6.8, 2H), 3.15 (t, J=7, 2H), 2.92 (t, J=6.2, 2H), 2.58 (t, J=7.7, 2H), 2.1 (t, J=7.5, 2H), 1.8-1.76 (m), 1.7-1.56 (m), 1.52-1.44 (m), 1.44-1.26 (m), 0.91 (t, J=7, 3H). ³¹P-NMR (CD₃OD): 9.05. MALDI Tof mass spec.:3599.32 (M+Na+), Calc'd: C₁₉₆H₃₄₂N₂₇O₂₄P₃: 3577.92.

### Exemplary Polyphosphazene Design Strategies and Experimental Outline

Amphoteric phosphazenes with both the cationic (polyamino) and anionic(polycarboxylic) components built into it may result in an adjuvant which has a synergistic combination of the immunogenic properties of polyamines and polyanionic phosphazene substituent as the amino (cationic) component will complement the acidic (anionic) component. This may also satisfy the observed requirements for formulation with PCEP. Protein based antigens may contain anionic, cationic as well as hydrophobic domains and may associate effectively with an adjuvant which has similar characteristics. As a consequence of the amphipathic nature of fatty acids they may arrange themselves into spherical micelle forms in aqueous solutions.

Amphipathic compounds may form micelles and they may arrange themselves into ordered forms (i.e. self-assemble), in aqueous solutions. Amphoteric-amphipathic molecules that contain both amino and carboxyl functional groups (polar head groups) as well as a lipid (tail) component may be induced to self-assemble under the right conditions in the presence of antigens and metal ions to form microparticles, thereby encapsulating the antigen.

Biologically, an amphoteric/amphipathic adjuvant may be more versatile. It may associate with a wider variety of antigens. It may tolerate a wider pH range and retain a given structure over a broader pH range. Antigens may be transported across cells through varying pH changes depending on the location in the cellular compartment. An antigen encapsulated with such an amphoteric compound could survive such pH changes and not be prematurely released or degraded as it is transported through changing cellular environment.

An adjuvant that is anionic may associate well with cationic antigens, and one that is cationic (polyamino analog) may associate effectively with anionic antigens by ionic interaction of opposite charges and one with a poly hydroxy component may interact effectively with carbohydrate antigens through hydrogen bonding.

One possible characteristic of the designed ligands is they contain both hydrophilic regions (polar head groups) as well as hydrophobic regions (the long hydrophobic chain). These adjuvants may be versatile and be applicable to protein, deoxyribonucleic acid, (DNA), ribonucleic acids, (RNA) and carbohydrate based antigens and therefore to bacterial and viral antigens.

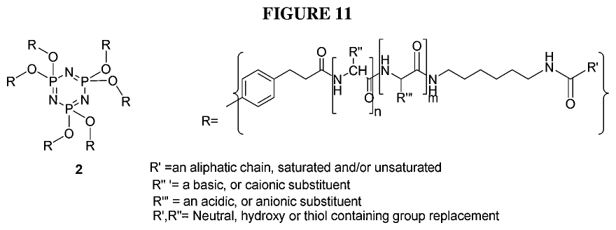

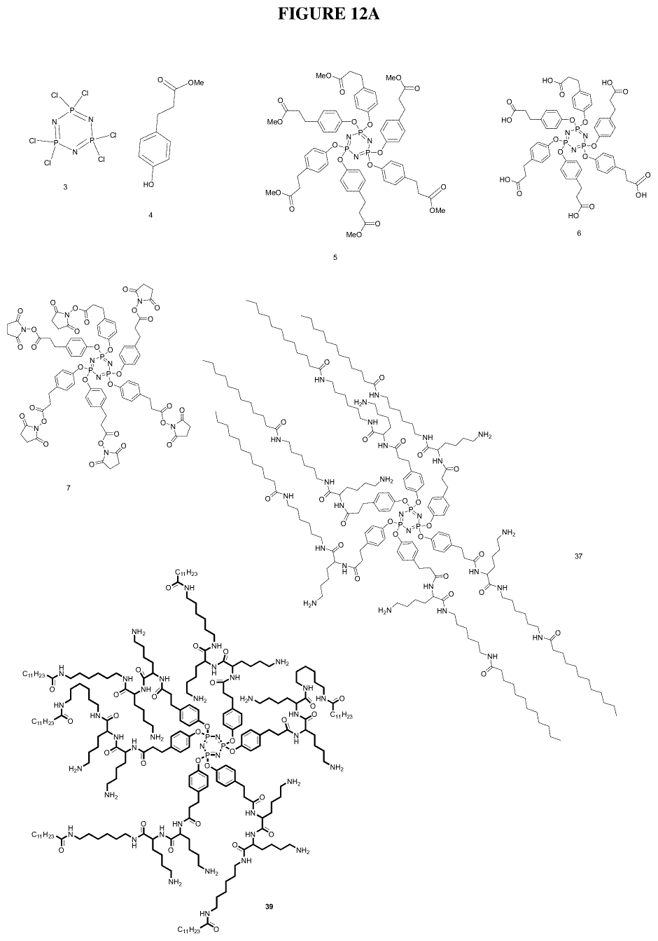

With these predetermined requirements taken into consideration, (ligands) that have hydrophilic head groups and a hydrophobic tail (FIG. 11) were designed and synthesized. All of these useful properties (see above) were considered and incorporated into the ligand design.

Cyclophosphazene was used as the core platform onto which the amphoteric, amphipathic ligands were assembled to give the poly amino, poly carboxylic, and poly lipid characteristics. The general structure, of these adjuvants for evaluation is shown in FIG. 11, compound 2. By varying the values of n and m, these compounds may have potential net positive or negative charge or be neutral. These compounds may have hydrophilic head and hydrophobic tail for self-assembly and formation of ordered structures with a wide variety of antigens.

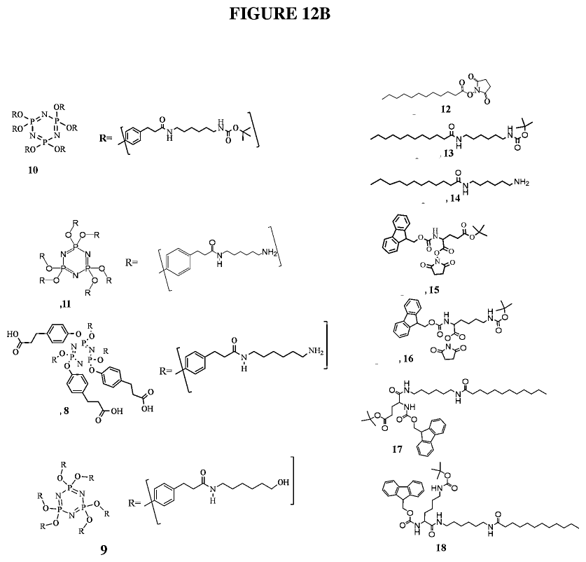

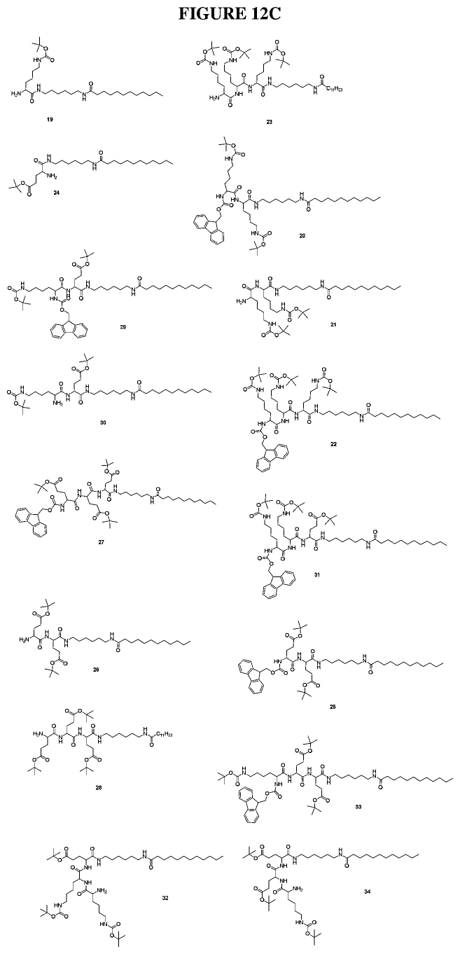

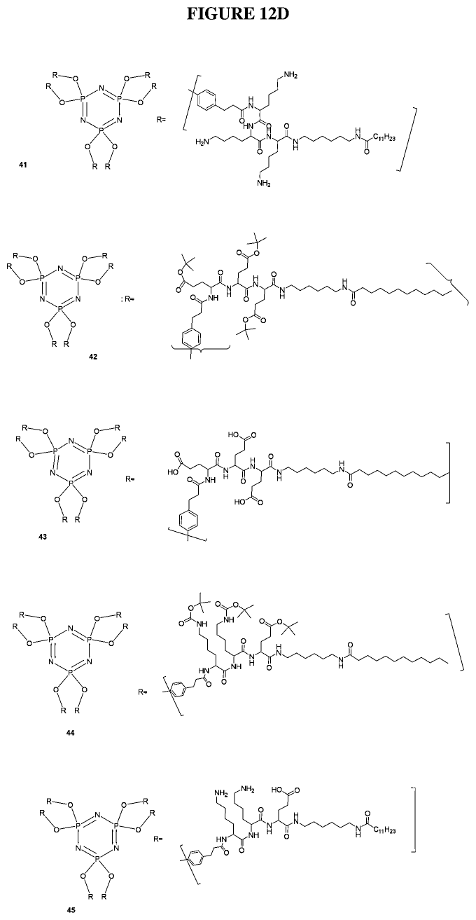

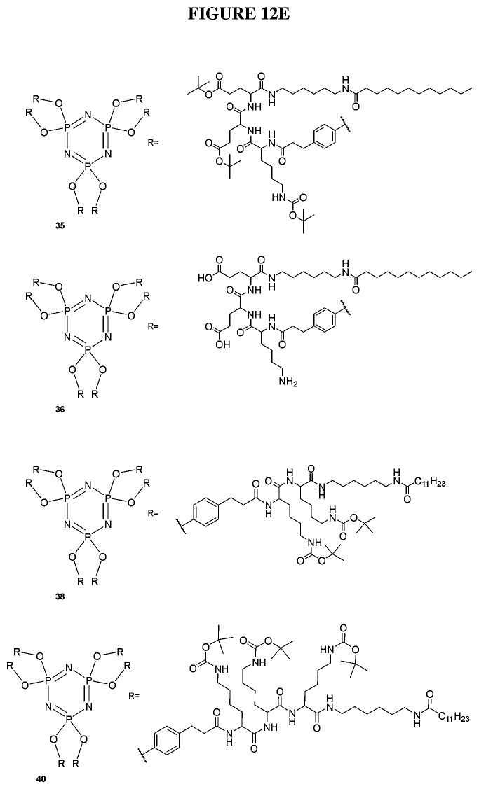

Referring to FIGS. 10 and 11, the acid 6 was converted to the hexa-N-oxysuccinimide ester derivative, 7. Reaction of 7 with the appropriate reagent to afford the basic compound 11 and the neutral compound 9 demonstrated that 7 would be very useful for the coupling of various ligands. The designed, protected amphipathic ligands 19, 21, 23, 28, 32, 34 (FIG. 12) were synthesized. The choice of substrates for the assembly of the ligands took into consideration the overall cost of intended use of the end product. A fatty acid was first coupled to a bi-functional linker, hexanediamine (HDA), to provide a functional group for further elaboration of the ligand. In using fatty acids as the hydrophobic component, the lipid character of the ligand can be readily modified by altering the composition and length of the lipid chain. (L)-amino acids were chosen for the hydrophilic and charge components of the ligands. These components were chosen so that the metabolic by products of the adjuvant would be naturally occurring non-toxic amino acids and fatty acids.

The ligands were coupled to the intermediate compound 7 to produce the compounds to be evaluated. The synthetic adjuvants shown in FIG. 11, 2 are expected to have predetermined properties by varying the ratios of m and n for the basic (R″) and acidic (R′″) residues. As a proof of concept some combinations were prepared.

Referring to FIGS. 10 and 11, for n=1, m=0, the product, compound 37, may be amphipathic with basic (cationic) properties. For n=2, m=0, the product, compound 39 is amphipathic with basic (cationic) properties, for n=3, m=0, the product, compound 41, is amphipathic with basic (cationic) properties. For n=1 and m=2, the product compound 36 is amphoteric, amphipathic with a net acidic (anionic) properties. For n=2 and m=1 the product, compound 45, is amphoteric, amphipathic with a net basic (cationic) properties. For n=O and m=3, the product compound 43, is amphipathic with acidic (anionic) properties. For future work the effects of increased lipid chain length, and a changing (R″) or (R′″) into a hydroxyl containing substituent should be studied. This could lead to the a versatile amphoteric, amphipathic adjuvant which can be used to formulate bacterial, viral, deoxyribonucleic acid, (DNA), ribonucleic acid (RNA), carbohydrate and protein based vaccines and based The details of the synthesis are provided in the experimental section.

### Characterization Data

### Compound 8

To the cyclophosphazene acid, compound 6 (250 mg, 0.23 mmol) in dichloromethane 5 ml, DMF, 0.7 ml was added N,N′-diisopropylcarbodimide (141 μL, 0.92 mmol) and stirred for 2 hr. N-t-Boc-hexylaminocarbamate hydrochloride (232.4 mg, 0.92 mmol) and diisopropylethylamine (350 μl) was added and stirred for 4 hr. Trifluoroacetic acid, 3 ml was added and stirred for 1 hr. The solvent was evaporated under vacuum and diethyl ether added to the residue. The precipitated product was filtered. The precipitate was dissolved in water containing 10% acetonitrile and filtered. The filtrate was lyophilized to give the crude product, HPLC analysis showed presence of a mixture of products. Purification by hplc afforded the trisubstituted product, compound 8 (37% yield). MALDI ToF mass spectrum: compound 8, 1420.5, Calc'd: C72H96N9O15P3:1419.62 and disubstituted product compound 8a, 1322.3, Calc'd: C66H82N7O16P3: 1321.50

### Compound 9-3,3′,3″,3′″,3″″,3′″″-(((1,3,5,2l5,4l5,6l5-triazatriphosphinine-2,2,4,4,6,6-hexayl)hexakis(oxy))hexakis(benzene-4,1-diyl))hexakis(N-(6-hydroxyhexyl)propanamide)

To cyclotriphosphazene N-hydroxysuccinimidylester, compound 7, (1.0 g, 0.59 mmol) in dichloromethane, 20 ml and DMF, 1 ml was added 6.2 equivalents of 6-aminohexan-1-ol (421.27 mg, 3.59 mmol) and stirred at room temperature for 18 hr. Diethyl ether was added to the reaction mixture and the precipitated material filtered to give a crude product to give The crude product was dissolved in warm acetonitrile (1% ethanol) and allowed to cool at 4° C. The crystallized product was filtered. The crystallized product was filtered and dried to afford compound 9 (448 mg, 44.12%). MALDI ToF mass spectrum: 1722.2, 1744.1 (M+Na+); Calc'd:C90H132N9O18P3: 1719.89.

### Compound 10—Hexa-tert-butyl (((3,3′,3″,3′,3″″,3′″″-(((1,3,5,2l5,4l5,6l5-triazatriphosphinine-2,2,4,4,6,6-hexayl)hexakis(oxy))hexakis(benzene-4,1-diyl))hexakis(propanoyl))hexakis(azanediyl))hexakis(hexane-6,1-diyl))hexacarbamate

To crude Hexakis-(N-oxysuccinimidyl-4-oxyphenylpropionate)cyclotriphosphazene, compound 7, (1.02 g, 0.6 mmol) in dichloromethane, 20 ml, was added 6.2 equivalents of tert-butyl-N-(6-aminohexylcarbamate) hydrochloride (91 mg, 0.37 mmol), and diisopropylethylamine, (0.37 mmol, 370 uL of IM solution in DMF) and stirred at room temperature for 22 hr. Diethyl ether was added to precipitate the product, compound 10. The crude product was filtered and the product crystallized from acetonitrile-ethyl acetate solution to give compound 10 (1.2 g, 86.3%). Maldi ToF mass spectrum. Molecular weight: 2339.6 (M+Na+). 1H-NMR: δ (CDCl₃): 6.9 (d, J=8.4), 6.7 (d, J=8.4), 2.44 (t, J=7.8), 2.89 (t, J=7.8), 1.42 (s).

### Compound 11-3,3′,3″,3′″,3″″,3′″″-(((1,3,5,2l5,4l5,6l5-triazatriphosphinine-2,2,4,4,6,6-hexayl)hexakis(oxy))hexakis(benzene-4,1-diyl))hexakis(N-(6-aminohexyl)propanamide)

Hexakis-[(N-6-tert-butyloxycarbonyl-hexyl)-4-oxyphenylpropionamide]cyclotriphosphazene (1.2 g, 0.52 mmol), compound 10, was dissolved in trifluoroacetic acid 5 ml and stirred at room temperature for 2 h. The solvent was evaporated under vacuum and the residue dissolved in water and 1 mmol of hydrochloric acid added and the sample lyophilized to give a solid, compound 11. Maldi ToF mass spectrum. Mol. Wt.: 1716. 1H-NMR: δ (D2O): 6.96 (d, J=8.4, 2H), 6.62 (d, J=8.4, 2H), 2.84 (t, J=6.9, 2H), 2.89 (t, J=7.1, 2H), 2.38 (t, J=7.1, 2H), 2.77 (q, J=7.1, 6.5, 2H), 1.54 (quintet, J=7.4, 2H), 1.42 (quintet, J=7.6, 2H), 1.2 (quintet, J=7.5, 2H). MALDI ToF mass spectrum:1716.10; Calc'd: C90H138N15O12P3: 1713.99.

### Compound 12—2,5-dioxopyrrolidin-1-yl dodecanoate

Dodecanoic acid (12.08 g, 60.40 mmol) and N-hydroxysuccinimide (7.6 g, 66.4 mmol) was dissolved in dichloromethane, (60 ml) and dimethylformamide (4 ml). To the solution was dicyclohexylcarbodimide solution in dichloromethane (60 ml of 1.0 M solution, 60 mmol) and stirred at room temperature for 18 h. The precipitated dicyclohexylurea was filtered off and the solvent evaporated from the filtrate under vacuum. The residue was dissolved in warm ethanol and kept at 4° C. overnight. The crystallized product, compound 12 was filtered and dried under vacuum (16.6 g, 92.58% yield). 1H-NMR: δ (CDCl3): 2.86, 2.85, 2.62 (t, J=7.2), 1.75 (quintet, J=7.2), 1.41 (quintet, J=7.2), 1.27-1.37 (m), 0.89 (t, J=7.2),

### Compound 13—Tert-butyl(6-dodecanamidohexyl)carbamate

2,5-dioxopyrrolidin-1-yl dodecanoate, compound 12, (6.0 g, 20.21 mmol) was dissolved in dichloromethane, 20 ml. Tert-butyl(6aminohexyl carbamate hydrochloride 5.0 g, 19.78 mmol) was added and stirred at room temperature. N,N-diisopropylethylamine (9.8 ml, 56.07 mmol) was added and stirred for 20 hr. insoluble precipitated material was filtered off and discarded. The solvent was evaporated from the filtrate under vacuum and the residue dissolved in warm n-hexane:diethylether (1:1). The solution was decanted from an oily insoluble residue. The solution was allowed to cool and kept at −40° C. overnight. The precipitated product was filtered and dried under vacuum to give compound 13 (7.46 g, 96.89%). 1H-NMR: δ (CDCl3): 0.91, 2.18, 3.25, 3.12, 1.48, 1.26-1.64. MALDI ToF mass spectrum: 421.4 (M+Na+); Calc'd: C₂₃H₄₆N₂O₃: 398.35.

### Compound 14—N-(6-aminohexyl)dodecanamide

To tert-butyl(6-dodecanamidohexyl)carbamate, compound 13 (7.40 g, 18.56 mmol) was added trifluoroacetic acid, (8.0 ml) and stirred at room temperature for 2 hr. The solvent was evaporated under vacuum and the residue dissolved in hot acetonitrile and cooled at 4° C. The precipitated product was filtered off. The filtrate was concentrated and diethyl ether added and kept at 4° C. The precipitated crude product was filtered. The crude product was suspended in 0.1M sodium bicarbonate solution, filtered and washed with cold water. The filtered solid product was dissolved in warm acetonitrile and kept at 4° C. The precipitated free amino compound was filtered and dried affording compound 14 (4.92, 88.90%). 1H-NMR: δ (CD3OD): 0.92 (t, J=7.2), 2.19 (t, J=7.5), 3.18 (t, J=7.2), 2.92 (t, J=7.5), 1.31-1.68. MALDI ToF mass spectrum: Mol. Wt. 321.2 (M+Na+); Calc'd: C₁₈H₃₈N₂O: 298.30.

### Compound 15—5-(tert-butyl) 1-(2,5-dioxopyrrolidin-1-yl) (((9H-fluoren-9-yl)methoxy)carbonyl)-L-glutamate

To N-α-Fmoc-L-Glutamic acid (OtBu)-OH (4.25 g, 10 mmol) and N-hydroxysuccicnimide (1.42 g, 12.37 mmol) in dichloromethane (40 ml), dimethylformamide, 1 mL was added dicyclohexylcarbodiimide, (1 mL) of 1.0 M solution in dichloromethane) and stirred at room temperature for 20 hr. Insoluble dicyclohexylurea byproduct was filtered off and the solvent evaporated from the filtrate under vacuum to give a crude product. The product was crystallized from ethyl acetate:diethyl ether solution of the crude product compound 15, (4.31 g, 82.7%). 1H NMR: 8 (CDCl3): 7.78 (d, J=7.2), 7.61 (t, J=6.6), 7.42 (t, J=7.2), 5.7 (d, J=7.8), 4.8 (td, J=7.8, 4.8), 4.26 (t, J=7.2), 2.86 (m), 2.5 (m), 2.34 (m), 2.19 (m), 1.48 (s). MALDI ToF mass spectrum: Mol. Wt. 561.2 (M+K+); Calc'd: C28H30N2O₈: 522.2.

### Compound 16—2,5-dioxopyrrolidin-1-yl N2-(((9H-fluoren-9-yl)methoxy)carbonyl)-N6-(tert-butoxycarbonyl)-L-lysinate

To N-α-Fmoc-L-Lysine(N-ε-t-Boc)-OH (6.0 g, 12.37 mmol) and N-hydroxysucccinimide (1.48 g, 12.37 mmol) in dichloromethane, 40 ml, dimethylformamide, 3 mL was added dicyclohexylcarbodiimide, 13 mL of 1.0 M solution in dichloromethane and stirred at room temperature for 20 hr. Insoluble dicyclohexylurea byproduct was filtered off and the solvent evaporated from the filtrate under vacuum to give a crude product. The product was crystallized from ethyl acetate solution of the crude product, compound 16 (6.61 g, 91.42%).

1H-NMR: δ (CDCl3) 7.78 (d, J=7.2), 7.62 (t, J=6), 7.4 (t, J=7.5), 5.52 (d, J=7.8) 4.75, 4.47, 4.26 (t, J=7.2), 3.17, 2.86, 2.03, 1.94, 1.51, 1.45 (s). Maldi Tof Mass spec: 565.24. MALDI ToF mass spectrum: Mol. Wt. 588.3 (M+Na+); Calc'd: C30H35N3O8: 565.24.

### Compound 17—tert-butyl 4-((((9H-fluoren-9-yl)methoxy)carbonyl)amino)-5-((6-dodecanamidohexyl)amino)-5-oxopentanoate

To N-α-Fmoc-L-Glutamic acid (OtBu)N-Hydroxysuccinimide ester, compound 15 (3.02 g, 5.7 mmol) in dichloromethane, 20 mL was added N(6-aminohexyl)dodecanamide, (1.71 g, 5.7 mmol). Dimethylformamide, 2 ml was added to aid dissolving the reactants. Diisopropylethylamine (1.0 mL, 5.7 mmol) was added and stirred at room temperature and the reaction monitored by hplc until completion of the reaction. Dichloromethane was evaporated under vacuum and the residue dissolved in warm acetonitrile and allowed to cool. The precipitated product was filtered and washed with cold acetonitrile and dried to afford compound 17 (3.55 g, 87.06%)

1H-NMR: δ (CD3OD) 7.81 (d, J=7.5, 2H), 7.68 (t, J=6.7, 2H), 7.4 (t, J=7.4, 2H), 7.33 (t, J=7.4), 4.4 (J=7.8, 6.9, 2H), 4.249 (t, J=6.7, 1H), 4.08 (dd, J=9.3, 5.7, 1H), 3.18 (t, J=6.9, 2H), 3.15 (t, J=6.9, 2H), 2.31 (t, J=7.4, 2H), 2.16 (t, J=7.4, 2H), 1.43 (s), 0.90 (t, J=6.9, 3H). Maldi Tof Mass spec 728.5 (M+Na+); MALDI ToF mass spectrum: Mol. Wt. 728.6 (M+Na+); Calc'd: C42H63N3O6: 705.47.

### Compound 18—(9H-fluoren-9-yl)methyl tert-butyl (6-((6-dodecanamidohexyl)amino)-6-oxohexane-1,5-diyl)dicarbamate

To N-α-Fmoc-L-Lysine(N-ε-t-Boc)-N-Hydroxysuccinimide ester (compound 16, 5.0 g, 8.84 mmol) in dichloromethane, 20 mL, ethanol 10 mL was added N(6-aminohexyl)dodecanamide (2.64 g. 8.84 mmol) and diisopropylethylamine, 3.1 ml. The solution was stirred at room temperature for 4 hr. The solvent was evaporated under vacuum. The residue was dissolved in hot acetonitrile and allowed to cool at 4° C. The precipitated product was filtered and washed with cold acetonitrile affording compound 18 (5.30 g, 80.3% yield).

1H-NMR: δ (CDCl3): 7.79 (d, J=7.5), 7.53 (d, J=6.87), 7.43 (t, J=7.39), 7.34 (t, J=7.27), 6.32, 5.65, 4.22, 4.15, 3.23, 3.13, 2.16 (t, J=7.63), 0.9 (t, J=6.79). MALDI ToF mass spectrum: Mol. Wt. 771.6 (M+Na+); Calc'd: C44H65N4O6: 748.51.

### Compound 19—tert-butyl (5-amino-6-((6-dodecanamidohexyl)amino)-6-oxohexyl)carbamate

To N-α-Fmoc-L-Lysine(N-ε-t-Boc)-HDA-C12, (Compound 18) (5.0 g, 6.65 mmol) in acetonitrile:methanol (1:1), 10 mL, dimethylformamide, 800 μL was warmed to dissolve and piperidine 1.5 mL was added and stirred at room temperature for 2 hr. The solvent was evaporated under vacuum and the residue dissolved in methanol and kept at 4° C. for 48 hr. The precipitated crystalline byproduct was filtered off. The solvent was evaporated from the filtrate to afford a crude product. The crude product was dissolved in hot acetonitrile containing-2% methanol and kept at 4° C. The precipitated product was filtered and dried. The crude product was suspended in diethyl ether, filtered and washed with diethyl ether affording Compound 19 (3.1 g, 88.57% yield).

1H-NMR: δ (DMSO-d6) 7.76 (t, J=5.73, 1H), 7.7 (t, J=5.46, 1H), 6.74 (t, J=5.28, 1H), 3.54 (q, J=5.9), 3.0 (q, J=6), 2.87 (q, J=6), 2.02 (t, J=7.38), 1.52-1.45 (m), 1.37 (s), 1.29-1.18 (bs), 0.85 (t, J=6.99). MALDI ToF mass spectrum: Mol. Wt. 549.5 (M+Na+); Calc'd: C28H58N4O4: 526.45.

### Compound 20—(9H-fluoren-9-yl)methyl tert-butyl (6-((2,2-dimethyl-4,11,20-trioxo-3-oxa-5,12,19-triazahentriacontan-10-yl)amino)-6-oxohexane-1,5-diyl)dicarbamate

To N-α-Fmoc-L-Lysine(N-ε-t-Boc)-N-Hydroxysuccinimide ester, compound 16, (1.0 g 1.7 mmol) in chloroform, 20 mL, dimethylformamide, 800 μL warmed to dissolve was added L-Lysine(N-ε-t-Boc)-HDA-C12, compound 19, (926.50 mg, 1.76 mmol) and stirred at room temperature for 2 hr. The solvent was evaporated under vacuum and the residue dissolved in hot acetonitrile and allowed to cool. The precipitated product was filtered and washed with cold acetonitrile to give the product, compound 20 (1.49 g, 86,63%).

1H-NMR: δ (DMSO-d6): 7.89 (d, J=7.56), 7.82 (d, J=7.62), 7.72 (d, J=8.58), 7.47 (d, J=8.04), 7.41 (d, J=7.44), 7.33 (t, J=7.4), 6.75 (d, J=5.28), 6.70 (d, J=5.19), 4.30 (q, J=7.32), 4.22 (q, J=7.03), 4.16 (q, J=7.06), 4.14 (q, J=7.8), 3.96 (q, J=8.52), 0.84 (t, J=6.96). MALDI ToF mass spectrum: 999.65 (M+Na+); Calc'd: C55H88N6O9: 976.66.

### Compound 21—tert-butyl (5-amino-6-((6-dodecanamidohexyl)amino)-6-oxohexyl)carbamate

To compound 20, (1.4 g, 1.52 mmol) in methanol:acetonitrile (1:1), 20 ml, dimethylformamide 1.0 mL warmed to dissolve. Piperidine, 500 μL was added and stirred at room temperature for 2 hr. The solvent was evaporated and the residue dissolved in warm acetonitrile, allowed to cool and kept at 4° C. The precipitated product was filtered and dissolved in warm methanol and kept at 4° C. The insoluble material was filtered off. The solvent was evaporated from the filtrate under vacuum.

The residue from the filtrate was dissolved in warm acetonitrile and allowed to cool at 4° C. The product precipitated out of solution and was filtered and dried under vacuum. The crude product was suspended in diethyl ether, filtered and washed with diethyl ether affording, compound 21 (1.05 g, 91.30% yield).

1H-NMR: δ (DMSO-d6) 7.93 (d, J=7.68), 7.90 (t, J=5.49), 7.70 (t, J=5.55), 4.18 (q, J=5.82), 3.11 (dd, J=2.7, 3.4), 3.05 (dt, J=6.5, 6.7), 3.0 (m), 2.87 (d,d,d, J=6.5), 2.5 (dt, J=7, 3.6), 2.02 (t, J=7.3), (1.41-1.62), 1.36 (s), 1.365 (s), 1.23 (broad), 0.85 (t, J=6.9). MALDI ToF mass spectrum: 778.66 (M+Na+); Calc'd: C40H78N6O7: 754.59.

### Compound 22—(9H-fluoren-9-yl)methyl tert-butyl (8,11-bis(4-((tert-butoxycarbonyl)amino)butyl)-6,9,12,21-tetraoxo-7,10,13,20-tetraazadotriacontane-1,5-diyl)dicarbamate

To N-α-Fmoc-L-Lysine(N-ε-t-Boc)-N-Hydroxysuccinimide ester, compound 16, (900 mg, 1.59 μmol) and in dichloromethane, 20 mL, dimethylformamide, 800 μL warmed to dissolve was added L-[L-Lysine(N-ε-t-Boc)]2-HDA-C12, compound 21, (1.02 g, 1.35 μmol) and stirred at room temperature for 2 hr. The solvent was evaporated under vacuum and the residue dissolved in hot acetonitrile and allowed to cool. The precipitated product was filtered and washed with cold acetonitrile affording compound 22 (1.32 g, 82.5% yield).

1H-NMR: δ (CD3OD) 7.82 (d, J=7.5), 7.7 (d, J=6.5), 7.41 (t, J=7.4), 7.33 (t, J=7.4), 4.43 (t, J=6.9), 4.37 (t, J=8.4), 4.25 (t, J=6.3), 4.05, 3.19 (t, J=6.0), 3.16 (t, J=6.9), 3.06 (t, J=6.69), 2.16 (t, J=7.5), 0.91 (t, J=6.9). MALDI ToF mass spectrum: 1227.84 (M+Na+); Calc'd: C66H108N8O12: 1204.81.

### Compound 23—Tert-butyl (5-(2-amino-6-((tert-butoxycarbonyl)amino)hexanamido)-6-((2,2-dimethyl-4,11,20-trioxo-3-oxa-5,12,19-triazahentriacontan-10-yl)amino)-6-oxohexyl)carbamate

N-FMoc[L-Lysine(N-ε-t-Boc)]3-HDA-C12, compound 22, (1.32 g, 1.09 μmol) was dissolved in methanol:acetonitrile (1:1), 30 mL with warming. Piperidine, 1.5 mL was added and stirred at room temperature for 2 hr, The solvent was evaporated and the residue dissolved in warm methanol and allowed to cool at 4° C. The insoluble by product which precipitated was filtered off. The solvent was evaporated from the filtrate and the residue dissolved in warm acetonitrile and kept at 4° C. The precipitated product was filtered and dried to give the product, compound 23 (941.20 mg, 87.42% yield).

1H-NMR: δ (CD3OD) 4.33 (t, J=7.98), 4.28, 3.36 (t, J=5.13), 3.21, 3.17 (t, J=7.23), 3.04 (t, J=9.82), 2.18 (t, J=7.53), 0.92 (t, J=6.96). Maldi Tof Mass spec: 1005.6857 (M+Na+); MALDI ToF mass spectrum: 1005.69 (M+Na+); Calc'd: C51H198N8010: 982.74.

### Compound 24—tert-butyl 4-amino-5-((6-dodecanamidohexyl)amino)-5-oxopentanoate

Compound 17 (2.0 g, 2.83 mmol was dissolved in acetonitrile:methanol (1:1), (15 mL) dimethylformamide, (400 μL). Piperidine, 308 μL was added and stirred at room temperature for 2 hr. The solvent was evaporated under vacuum and the residue dissolved in warm acetonitrile and cooled at 4° C. The precipitated product was filtered and washed with cold acetonitrile and dried under vacuum. The filtrate was concentrated and cooled again to precipitate a second crop of the product. Purified by HPLC. Compound 24, (1.0 g, 72.99% yield).

1H-NMR: δ (CDCl3) 5.6, 2.62, J=1.46 (s), 0.90 (t, J=6.72). MALDI ToF mass spectrum: 506.4 (M+Na+); Calc'd: C₂₇H₅₃N₃O₄: 483.4.

### Compound 25: Tert-butyl 4-((((9H-fluoren-9-yl)methoxy)carbonyl)amino)-5-((5-(tert-butoxy)-1-((6-dodecanamidohexyl)amino)-1,5-dioxopentan-2-yl)amino)-5-oxopentanoate

To N-α-Fmoc-L-Glutamic acid (OtBu)N-Hydroxysuccinimide, ester compound 15, (0.95 g, 1.80 mmol) was added Glu(OtBu)-HDA-C₁₂, compound 24, (880 mg, 1.80 mmol) in dichloromethane (20 mL) and stirred at room temperature for 12 hr. The solution was filtered and the solvent evaporated from the filtrate. The residue from the filtrate was dissolved in warm acetonitrile and allowed to cool at 4° C. The precipitated product was filtered, washed with cold acetonitrile and dried under vacuum to give, compound 25 (1.5 g, 93.75% yield).

1H-NMR: δ (DMSO-d6), 7.93 (d, J=7.98, 1H), 7.89 (d, J=7.5, 2H), 7.82 (t, J=5.4, 1H), 7.71 (t, J=2.5, 1H), 7.69. MALDI ToF mass spectrum: 913.57 (M+Na+); Calc'd: C51H78N4O9: 890.58.

### Compound 26—Tert-butyl 4-amino-5-((5-(tert-butoxy)-1-((6-dodecanamidohexyl)amino)-1,5-dioxopentan-2-yl)amino)-5-oxopentanoate

Compound 25, (1.62 g, 1.81 mmol) was dissolved in acetonitrile:methanol (1:1), 20 mL, dimethylformamide, (1 mL) and piperidine (1 mL) added and stirred at room temperature for 2 hr. The solvent was evaporated under vacuum and the residue dissolved in warm methanol and kept at 4° C. overnight. The precipitated material was filtered off. The solvent was evaporated from the filtrate under vacuum and the residue dissolved in acetonitrile and kept at 4° C. for a day. The precipitate is filtered and dried to give compound 26, (0.9 g, 74.31% yield).

1H-NMR: δ (DMSO-d6) 7.98, 7.91 (t, J=6.0), 7.7 (t, J=5.58), 4.23, 3.14, 2.01 (t, J=7.38), 1.38 (s), 0.84 (t, J=6.99). MALDI ToF mass spectrum: 691.53 (M+Na+), 731.57 (M+K+); Calc'd: C36H68N4O₇: 668.51.

### Compound 27—Tert-butyl 4-((((9H-fluoren-9-yl)methoxy)carbonyl)amino)-7,10-bis(3-(tert-butoxy)-3-oxopropyl)-5,8,11,20-tetraoxo-6,9,12,19-tetraazahentriacontanoate

To N-α-Fmoc-L-Glutamic acid (OtBu)N-Hydroxysuccinimide ester, compound 15, (1.17 g, 2.24 mmol) was added Glu(OtBu)]2-HDA-C₁₂, compound 26, (1.49 g, 2.24 mmol) in dichloromethane (20 mL) and stirred at room temperature for 12 hr. The solution was filtered and the solvent evaporated from the filtrate. The residue from the filtrate was dissolved in warm acetonitrile and allowed to cool at 4° C. The precipitated product was filtered, washed with cold acetonitrile and dried under vacuum to give compound 27 (2.38 g, 98.71% yield).

1H-NMR: δ (DMSO-d6), 8.05, 8.01, 7.93, 7.90, 7.72, 7.6, 7.33, 4.3, 4.27, 4,24, 4.20, 3.06, 3.00, 2.24, 2.22, 2.18, 2.02 (t, J=7.32), 1.38, 0.85 (t, J=6.84). MALDI ToF mass spectrum: 1098.77 M+Na+), 1114.7469 (M+K+). Calc'd: C60H93N5O12: 1075.68.

### Compound 28—tert-butyl 4-amino-7,10-bis(3-(tert-butoxy)-3-oxopropyl)-5,8,11,20-tetraoxo-6,9,12,19-tetraazahentriacontanoate

Compound 27, (1.24 g, 1.15 mmol) was dissolved in methanol:acetonitrile (1:1), (20 ml). Piperidine, 1.4 ml was added and stirred at room temperature for 2 hr and the solvent evaporated under vacuum. The residue was dissolved in diethyl ether, kept at 0° C. for 2 hr and centrifuged at 10,000 rpm in a centrifuge (ThermoFisher Sorvall Evolution RC) at 0° C. for 30 minutes. The supernatant was removed. The residue was re-dissolved in diethyl ether, cooled and centrifuged again. This process was repeated until the supernatant did not give a precipitate upon cooling. The final precipitate was dissolved in acetonitrile chilled to −80° C. and lyophilized to give the product, compound 28, 840.0 mg (85.45% yield).

1H-NMR: δ (DMSO-d6), 7.72, 7.71, 7.70, 4.25, 4.17, 3.49, 3.17, 2.02 (t, J=7.4), 1.38, 0.85 (t, J=7.0). MALDI ToF mass spectrum: 876.64 (M+Na+), 892.61 (M+K+); Calc'd: C45H83N5O10: 853.61.

### Compound 29—Tert-butyl 4-(2-((((9H-fluoren-9-yl)methoxy)carbonyl)amino)-6-((tert-butoxycarbonyl)amino)hexanamido)-5-((6-dodecanamidohexyl)amino)-5-oxopentanoate

N-α-Fmoc-Lysine(N-ε-t-Boc)-N hydroxysuccinimide ester, compound 16 (1.0 g, 1.76 mmol) in chloroform (20 mL), dimethylformamide, (500 μL) was warmed to dissolve. Compound 24 (850 mg, 1.76 mmol) in chloroform (5 mL), dimethylformamide (200 μL) was added. The solution was stirred at room temperature. The reaction was monitored by hplc until completion. The solvent was evaporated under vacuum and the residue dissolved in hot acetonitrile and allowed to cool. The precipitated product was filtered to give compound 29, (1.32 g, 80.29% yield).

1H-NMR: δ (DMSO-d6) 7.91, 7.89 (d, J=7.6), 7.69 (d, J=5.6), 7.5 (d, J=7.8), 7.41 (t, J=7.44), 7.33 (t, J=7.4), 4.23-4.3, 4.19, 3.06 (m), 2.98, 2.89 (t, J=6.6), 1.36 (s), 1.35 (s), 0.84 (t, J=6.9). Maldi Tof Mass spec: Mol. Wt MALDI ToF mass spec: 956.61 (M+Na+); Calc'd: C53H83N5O9: 933.62.

### Compound 30—Tert-butyl 4-(2-amino-6-((tert-butoxycarbonyl)amino)hexanamido)-5-((6-dodecanamidohexyl)amino)-5-oxopentanoate

Compound 29, (0.6 g, 0.64 mmol) dissolved in methanol:acetonitrile (1:1), (5 mL), dimethylformamide 200 μL) was added piperidine (200 μL) and stirred at room temperature for 2 hr. The solvent was evaporated under vacuum and the residue dissolved in methanol and kept at 4° C. The precipitated by product was filtered off. The residue was triturated with dichloromethane and kept at 4° C. for 24 hr and the precipitated product filtered, washed with cold dichloromethane and dried under vacuum to give compound 30, (410 mg, 72.69% yield).

1H-NMR: δ (CD3OD) 2.18 (t, J=7.4), 2.31 (t, J=6.5), 2.32 (t, J=8.6), 4.33, 3.36, 3.05, 1.44 (s), 1.46 (s), 0.91 (t, J=6.9). MALDI ToF mass spec: 734.49 (M+Na+); Calc'd: C38H73N5O7: 711.55.

### Compound 31—Tert-butyl 10-((((9H-fluoren-9-yl)methoxy)carbonyl)amino)-13-(4-((tert-butoxycarbonyl)amino)butyl)-16-((6-dodecanamidohexyl)carbamoyl)-2,2-dimethyl-4,11,14-trioxo-3-oxa-5,12,15-triazanonadecan-19-oate

To compound 30 (260 mg, 0.36 mmol) in dichloromethane (10 mL) was added N-α-Fmoc-L-Lysine(N-ε-t-Boc)-N-hydroxysuccinimide ester, compound 16 (207 mg, 0.136 mmol) and stirred at room temperature for 12 hr. The solvent was evaporated and the residue dissolved in hot acetonitrile, 5% methanol and allowed to cool. The product precipitated upon cooling to room temperature and was filtered and dried to give compound 31 (320.0 mg, 74.64% yield).

1H-NMR: δ (DMSO-d6) 7.96 (d, J=7.62), 7.90 (d, J=7.56), 7.85 (d, J=7.74), 7.79 (t, J=5.58), 7.7201 (t, J=6.66), 7.7216 (d, J=7.14), 7.41 (t, J=7.44) 7.33 (t, J=7.44), 6.76 (t, J=5.58), 6.72 (t, J=5.40), 4.29, 4.26, 4.22, 4.19, 4.18, 2.02 (t, J=7.41), 1.34 (s), 1.36 (s), 1.37 (s), 0.85 (t, J=7.02). MALDI ToF mass spec: 1184.71, (M+Na+), 1200.68 (M+K+); Calc'd: C64H103N7O12: 1161.77.

### Compound 32—tert-butyl 10-amino-13-(4-((tert-butoxycarbonyl)amino)butyl)-16-((6-dodecanamidohexyl)carbamoyl)-2,2-dimethyl-4,11,14-trioxo-3-oxa-5,12,15-triazanonadecan-19-oate

Compound 31, (510 mg, 0.43 mmol) was dissolved in merhanol:acetonitrile (1:1), 10 mL. Piperidine, 1 mL was added and stirred at room temperature for 2 hr. The solvent was evaporated under vacuum and the residue triturated with diethyl ether. The precipitated material was filtered and dried affording the product compound 32 (380 mg, 92.20% yield).

1H-NMR: δ (DMSO-d6), 8.00 (d, J=6.12), 7.98 (d, J=7.97), 7.80 (t, J=5.6), 7.71 (t, J=5.58), 7.89 (t, J=5.6), 6.75 (t, J=5.52), 4.23, 4.14, 3.12, 3.07, 2.02 (t, J=7.4), 1.38, 0.85 (t, J=6.99). MALDI ToF mass spec: 962.78, (M+Na+); Calc'd: C49H93N7O10: 939.70.

### Compound 33—Tert-butyl 10-((((9H-fluoren-9-yl)methoxy)carbonyl)amino)-13-(3-(tert-butoxy)-3-oxopropyl)-16-((6-dodecanamidohexyl)carbamoyl)-2,2-dimethyl-4,11,14-trioxo-3-oxa-5,12,15-triazanonadecan-19-oate

To compound 26 (320 mg, 0.48 mmol) dissolved in dichloromethane 20 mL, dimethylformamide, 500 μl was added compound 16 (297.6 mg, 0.53 mmol) and the reaction mixture was stirred at room temperature for 12 hr. The solvent was evaporated under vacuum and the residue triturated with diethyl ether. The precipitated product was filtered and washed with diethyl ether. The solid material was dissolved in hot acetonitrile and insoluble material filtered off. The filtrate was allowed to cool and the pure product precipitated out. The product was filtered and dried to give compound 33 (430 mg, 51.39% yield). MALDI ToF mass spec: 1141.73, (M+Na+); Calc'd: C62H98N6O12: 1118.72.

### Compound 34—Tert-butyl 10-amino-13-(3-(tert-butoxy)-3-oxopropyl)-16-((6-dodecanamidohexyl)carbamoyl)-2,2-dimethyl-4,11,14-trioxo-3-oxa-5,12,15-triazanonadecan-19-oate

Compound 33 (920 mg, 0.82 mmol) was dissolved in acetonitrile:methanol (1:1), (30 mL). Piperidine, 1 mL was added and stirred at room temperature for 2 hr. The solvent was evaporated under vacuum and the residue dissolved in methanol and cooled at 4° C. The precipitated byproduct was filtered off. The solvent was evaporated from the filtrate. The residue from the filtrate was suspended in diethyl ether. The precipitated material was filtered and washed with diethyl ether affording the product compound 34 (630.0 mg, 88.5% yield).

1H-NMR: δ (CD3OD) 4.34, 3.39 (t, J=5.82), 3.17 (t, J=7), 3.05 (t, J=6.8), 2.18 (t, J=7.5), 1.47 (s), 1.46 (s), 1.44 (s), 0.91 (t, J=6.99). Maldi Tof Mass spec: Mol. Wt.: MALDI ToF mass spec: 919.69, (M+Na+); Calc'd: C47H88N6O10: 896.66

### Compound 35

To compound 7 (50 mg, 29.3 μmol) dissolved in dichloromethane (20 mL), was added 7.6 equivalents of compound 34 (200 mg, 0.22 mmol). DMF, 0.5 ml was added and stirred at room temperature for 48 hr. The solvent was evaporated under reduced pressure and the residue dissolved in hot 90% acetonitrile, 10% methanol and allowed to cool until precipitation started and kept at 4° C. overnight. The precipitated product was filtered and dried to give compound 35 (125 mg, 66% yield). MALDI Tof mass spec. 6418.64; Calc'd.: C336H570N39O72P3: 6475.73.

### Compound 36

Compound 35 (125 mg, 19.3 μmol) was treated with tfa, 2 ml for 3 hr. The tfa was evaporated under vacuum and the residue dissolved in hot acetonitrile containing 10% methanol and allowed to cool. The precipitated product was filtered to give compound 36 (76 mg, 76.8% yield). MALDI Tof mass spec.: 5134.9; Calc'd: C258H426N39O60P3: 5127.3.

### Compound 38

To compound 7 (100 mg, 58.5 μmol) dissolved in dichloromethane (20 mL) was added compound 21 (300 mg, 0.397 mmol) and stirred at room temperature for 12 hr. Dichloromethane (20 mL) was added (to aid stirring) and stirred for a further 6 hr. The solvent was evaporated under vacuum and the residue dissolved in hot acetonitrile, and insoluble material filtered off. The filtrate was concentrated and the solution allowed to cool. The precipitate was filtered affording a product, compound 38 (261.3 mg, 80.4%). The crude product was used for the next step.

### Compound 39—N,N′,N″-((((2S,2′S,2″S)-2,2′,2″-(((2S,2′S,2″S)-2,2′,2″-((3,3′,3″-((((S)-4,6,6-tris(4-((5R,8S)-5,8-bis(4-aminobutyl)-3,6,9,18-tetraoxo-4,7,10,17-tetraazanonacosyl)phenoxy)-1,3,5,2l5,4l5,6l5-triazatriphosphinine-2,2,4-triyl)tris(oxy))tris(benzene-4,1-diyl))tris(propanoyl))tris(azanediyl))tris(6-aminohexanoyl))tris(azanediyl))tris(6-aminohexanoyl))tris(azanediyl))tris(hexane-6,1-diyl))tridodecanamide

To compound 38 (226 mg, 40 μmol) was added trifluoroacetic acid (3 ml) and stirred at room temperature or for 2 hr. The solvent was evaporated under vacuum. The residue was dissolved in warm acetonitrile containing 2% methanol, allowed to cool to room temperature and kept at 4° C. for 18 hr and the precipitate filtered to give the product, compound 39 (136.2 mg, 78.11% yield).

¹H-NMR: δ (CD₃OD) 7.11 (t, J=8.58), 6.84 (t, J=8.48), 4.301 (t, J=5.8), 4.306 (t, J=6.12), 3.16 (t, J=7.1), 4.31 (t, J=9.1), 4.301 (t, J=8.7), 2.95 (dt, J=7.5, 3.2), 2.91 (t, J=8.2), 2.57 (t, J=7.9), 0.91 (t, J=7). ³¹P-NMR: δ (CD₃OD), 8.82. MALDI Tof mass spec.: 4366.5674, (M+Na+); Calc'd: C234H414N39O29P3:4344.18.

### Compound 40

To compound 7 (170 mg, 99.50 μmol) dissolved in dichloromethane (20 mL), was added 7 equivalents of compound 23 (685.0 mg, 696.5 μmol) and stirred at room temperature for 24 hr, and additional dichloromethane (40 mL) was added to aid in maintaining reaction solution and stirred for another 48 hr. The solvent was evaporated under vacuum and the residue triturated with diethyl ether and kept at 4° C. overnight. The precipitated solid was filtered and dried under vacuum to give compound 40 (685 mg, 99.51% yield). The crude product was converted to compound 41.

### Compound 41—N,N′,N″,N′″,N″″,N′″″-((((1,3,5,2l5,4l5,6l5-triazatriphosphinine-2,2,4,4,6,6-hexayl)hexakis(oxy))hexakis(benzene-4,1-diyl))hexakis(5,8,11-tris(4-aminobutyl)-3,6,9,12-tetraoxo-4,7,10,13-tetraazanonadecane-1,19-diyl))hexadodecanamide

To the isolated crude product compound 40 (650.0 mg) was added trifluoroacetic acid (10 mL) and stirred at room temperature for 3 hr. The trifluoroacetic acid was evaporated under vacuum and the residue triturated with diethyl ether and kept at 4° C. overnight. The precipitated product was filtered to give compound 41 (460 mg, 95.70%).

1H-NMR: δ (D20) 7.11 (d, J=8.6), 6.85 (d, J=8.4), 4.32 (d, d, J=6.6, 3.1), 4.29, 3.0, 3.16 (t, J=7.1), 2.90, 2.56 (d, d, J=4.62, 3.3), 2.18 (t, J=7.56), 0.91 (t, J=7.02, 3H). 31P-NMR: δ (CD3OD), 8.75. MALDI Tof mass spec.:5141.65, (M+Na+); Calc'd: C270H486N51O36P3:5116.02.

### Compound 42

To compound 7 (77.0 mg, 45 μmol) was added 7 equivalents of compound 28 (272.3 mg, 318 μmol) in dichloromethane (20 mL) and stirred at room temperature for 20 hr. Diisopropylethylamine (300 μL) was added and the reaction solution stirred for a further 48 hr. The solvent was evaporated under vacuum and the residue triturated with diethyl ether and kept at 4° C. overnight. The precipitated solid was filtered and dried under vacuum to give the product compound 42 (260 mg, 94.12% yield).

### Compound 43

To compound 42 (260 mg, 42.35 μmol) was added 10 mL of trifluoroacetic acid and stirred at room temperature for 4 hr. The solvent was evaporated and the residue triturated with diethyl ether and kept at 4° C. overnight. The precipitated solid was filtered to and dried under vacuum to give the product, compound 43 (160 mg, 95.80% yield).

1H-NMR: δ (DMSO-d6), 8.09 (d, J=7.0), 7.94 (d, J=7.6), 7.78 (d, J=5.0), 7.71 (d, J=7.3), 7.11 (d, J=8.52), 6.78 (d, J=8.43), 4.26, 4.21, 4.17, 2.02 (t, J=7.41), 0.84 (t, J=6.99). 31P-NMR (CD3OD): 8.43 (major), 8.51 (minor). The minor peak could be due to incomplete reaction product shown in the mass spec. at mass 4494.99. MALDI-Tof mass spec.: 5164.31, 4536.60; Calc'd: C258H414N36O72P3: 5259.54.

### Compound 44

To compound 7 (98.62 mg, 57.70 μmol) was added 7 equivalents of compound 32 (380.0 mg, 404 μmol) and dissolved in dichloromethane (40 mL) and diisopropylethylamine (70 μL) added and stirred at room temperature for 72 hr. The solvent was evaporated under vacuum and the residue dissolved in hot acetonitrile and kept at 4° C. overnight. The precipitated solid was filtered and dried under vacuum to give the product compound 44 (350 mg, 0.64% yield).

1H-NMR: δ (DMSO-d6), 7.82 (d, J=7.56), 7.63, (d, 7.14), 7.38 (t, 7.47), 7.29 (t, J=7.44), 4.29 (d, J=7.14), 4.25 (d, J=8.2), 4.16, 3.95, 2.92 (t, J=7.41), 1.45, 0.81 (t, J=6.96). 31P-NMR: δ (DMSO-d6) 8.39.

### Compound 45

To compound 44 (310 mg, 46.50 μmol) was added 10 mL of trifluoroacetic acid and stirred at room temperature for 4 hr. The solvent was evaporated and the residue dissolved in hot acetonitrile-10% methanol and kept at 4° C.

The precipitated solid was filtered and dried under vacuum to give the product compound 45 (205 mg, 77.99%).

1H-NMR: δ (DMSO-d6), 8.15 (d, J=6.90), 8.11 (d, J=6.72, 1H), 7.92 (d, J=5.19, 1H), 7.86 (d, J=7.74, 1H), 7.77 (d, J=5.61, 1H), 7.15 (d, J=8.52, 2H), 6.83 (d, J=8.34, 2H), 4.23, 4.21, 2.03 (t, J=7.41, 2H), 0.85 (t, J=6.99, 3H). 31P-NMR: S (DMSO-d6) 8.18. MALDI-Tof mass spec.: 5152.26; Calcd:C264H492N45O48P3: 5118.38.

### Example 3—Oligomeric Polyphosphazenes

Molecules that contain both the carboxyl and amino functional groups be may modified to self assemble under the right conditions to create microparticles.

For example, self-assembly may occur in the presence of antigens and metal ions thereby trapping the antigen. It has been observed that anionic PCEP may be more effective as an adjuvant when a polyamino (cationic) compound is added to the formulation in the presence of metal ions. Antigens have also been encapsulated with the polyphosphazene, PCEP. It is worth noting that polyethylenediamines have been used as adjuvants and delivery vehicles for DNA and cytokines.

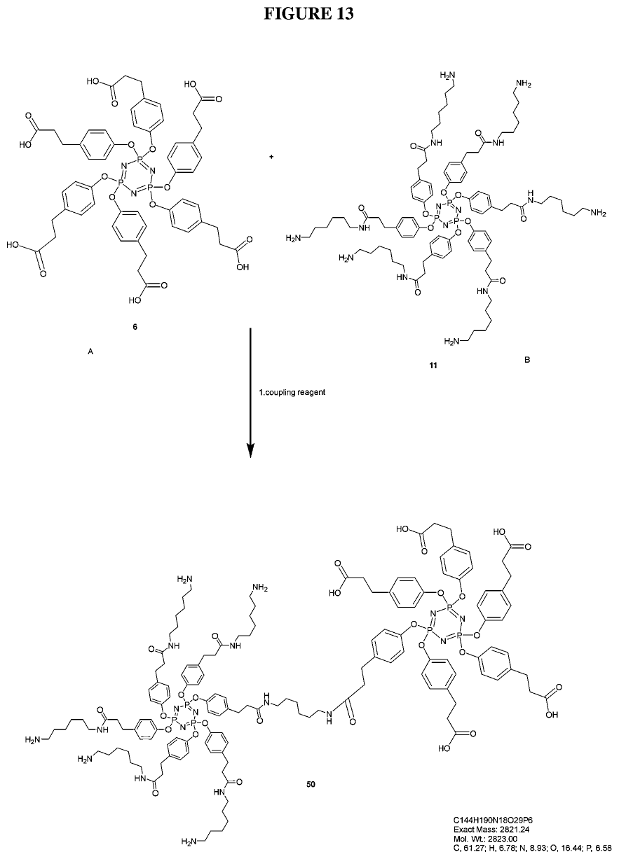

Referring to FIG. 13, reaction of compounds 6 and 11 may give the amphoteric, zwitterionic, compound 50 or analogues. Modification of the molecule 50 may provide oligomers, such as dimers, trimers or oligomers of itself or others, for example homodimers or heterodimers. Another option is to modify these cyclophosphazene-derived compounds with functionalized lipids to provide an anchor to hold in membranes. Formulation of vaccines with antigens and these derivatives and evaluation their ability to elicit immunological response was investigated.

Partial modification through the use of 2, 3, or 4 equivalents of the modifying reagent or the coupling reagent was investigated. For example, partial amidation of a suitable intermediate such as compounds 6 or 7.

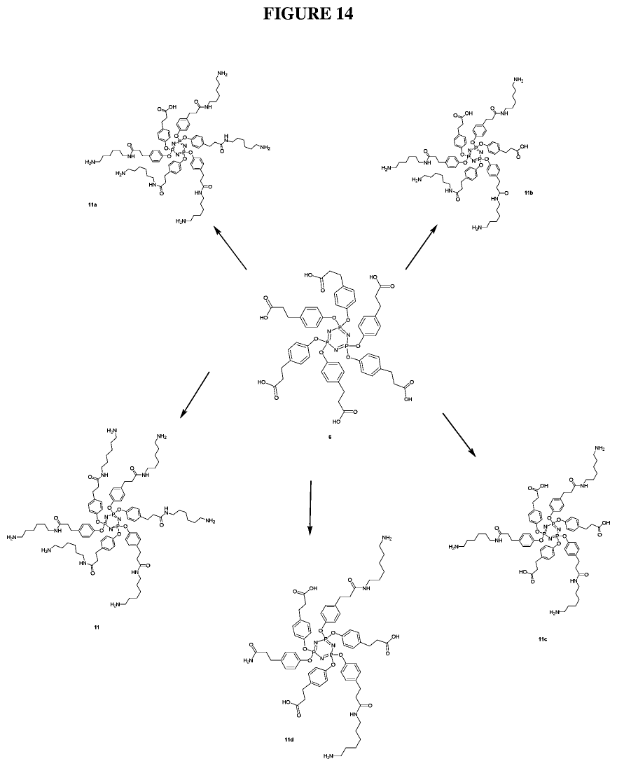

Referring to FIG. 14, X-ray structure of other substituted cyclophosphazene derivatives show that the aromatic ring substituent on alternate phosphorus atoms overlap with each other. This may indicate that three anhydride moieties could be generated if compound 6 is treated with 3 equivalents of a carbodiimide reagent, or other suitable couple reagent. Upon reaction with three equivalents of an amine reagent an amphoteric trisubstituted derivative 11c may be generated. Compounds 11a, 11b, 11c, and 11d may be mono-, di-, tri- and quad-substituted derivatives, respectively.

The modifications may affect the solubility of the compounds. These amino compounds may also complement the acidic molecules since the amino group provides a handle for further broader modifications.

Biologically an amphoteric zwitterionic molecule may be desirable since it can tolerate a wider pH range and retain a given structure over a broader pH range. Antigens may be transported through the cell through varying pH changes depending on the location in the cellular compartment. An antigen encapsulated with the amphoteric compound can survive such changes if need be and not be prematurely released or degraded.

The amino derivative 11 may be utilized to add in up to 6 more units of the acidic compound 6 by preparing an acid anhydride of 11(1-equivalent of a carbodiimide added to one equivalent of compound 11. This may form one anhydride per molecule as the major product for steric reasons—although it may prove to be difficult to accomplish. Hydroxy acids may form chelates and this might encapsulate other molecules such as antigens.

One objective of this study is to modify 11 to generate the hexa-amino derivative 6, and 6a-d. Reactions of 11 and 6 in various proportions (FIG. 13) to find the right conditions for the formation of compound 50 and its analogs was investigated. The product distribution may be controlled by reaction conditions temperature, rate of addition, and ratios of 11 and 6 added to the reaction mixture.

Compound 50 may be dimerized/oligomerized, or polymerised through intermolecular reaction of the carboxyl group on 7 with the amino group of another molecule on 50 or by activating any number of carboxyl groups on 11. This may generate an array of compounds which can then be screened for adjuvant activities. By using an excess of A (FIG. 13) the product can be made to have a net carboxylic acid group and therefore a net negative charge or by using an excess of B the product can be made to have a net amino group and therefore a net positive charge. By reacting the acid 6 with excess 6-amino-1-hexanol(protected), the product after deprotection may be a water soluble hydroxylated product 9 that can be transformed into other derivatives (A). Use 1 equivalent of compound 9 to partially esterify compound 6, 3 equivalents of to give a negatively charged (anionic) derivative. An immunogenic carbohydrate may be attached to this compound or any of the derivatives to make it more immunogenic and may be used in a formulation.

All citations are hereby incorporated by reference. In the event of conflicting information with statements between any reference to or incorporated herein, and the present disclosure, the present disclosure will act as the guiding authority.

The present invention has been described with regard to one or more embodiments. However, it will be apparent to persons skilled in the art that a number of variations and modifications can be made without departing from the scope of the invention as defined in the claims.

### REFERENCES

1. H HogenEsch, Vaccine (2002) 20 Suppl 3:S34-39
2. R Garg, L Latimer, E Simko, V Gerdts, A Potter, S van Drunen Littel-van den Hurk J. Gen. Virol. Induction of mucosal immunity and protection by intranasal immunization with a respiratory syncytial virus subunit vaccine formulation (2014) 95:301-306.
3. L G Payne, S A Jenkins, A L Woods, E M Grund, W E Geribo, J R Loebelenz, A K Andrianov, B E Roberts. Poly[di(carboxylatophenoxy)phosphazene](PCPP) is a potent immunoadjuvant for an influenza vaccine. Vaccine (1998) 16(1):92-98.
4. A K Andrianov, L G Payne. Protein release from polyphosphazene matrices. Adv. Drug. Deliv. Rev. (1998) 31:185-196
5. H M Geysen, R H Moelen, S J Barteling. Use of peptide synthesis to probe viral antigens for epitopes to a resolution of a single amino acid. Proc. Natl. Acad. Sci. USA (1984) 81:3998-4002.
6. H M Geysen, S J Rodda, T J Mason. A priori delineation of a peptide which mimics a discontinuous antigenic determinant. (1986)Molec. Immunol. 23:709-715.
7. T P Hopp, K R Woods. Prediction of protein antigenic determinants from amino acid sequences. Proc. Natl. Acad. Sci USA (1981) 78:3824-3828.
8. J Kyte, R F Doolittle. A simple method for displaying the hydropathic character of a protein. J. Mol. Biol. (1982) 157:105-132.
9. A K Andrianov, Y Y Svirkin, M P LeGolvan. Synthesis and biologically relevant properties of polyphosphazene polyacids. Biomacromolecules (2004) 5:1999-2006.
10. A K Andrianov, J Chen, M P LeGolvan. Poly(dichlorophosphazene) As a Precursor for Biologically Active Polyphosphazenes: Synthesis, Characterization, and Stabilization. Macromolecules (2004) 37:414
11. G Mutwiri, P Benjamin, H Soita, H Townsend, R Yost, B Roberts, A K Andrianov, L A Babiuk. Vaccine (2007) 25, 1204.
12. Boman, H. G. Peptide antibiotics and their role in innate immunity. Annu. Rev. Immunol. (1995) 13:61-92.
13. Boman, H. G. Gene-Encoded Peptide Antibiotics and the Concept of Innate Immunity: An Update Review. Scand. J. Immunol. (1998) 48:15-25.
14. W F Broekaert, F R G Terras, B P A Cammue, R W Osbom. Plant. Physiol. (1995) 108:1353-1358.
15. H Steiner, D Hultmark, Å Engström, H Bennich and H G Boman. Sequence and specificity of two antibacterial proteins involved in insect immunity, Nature (1981) 292:246-248.
16. T Ganz and R I Lehrer. Defensins. Curr. Opin. Immunol. (1994) 6:584-589.
17. R I Lehrer and T Ganz. Antimicrobial peptides in mammalian and insect host defence. Curr. Opin. Immunol. (1999) 11:23-27.
18. A J Ouellette and M E Selsted. Paneth cell defensins: endogenous peptide components of intestinal host defense, FASEB J. (1996) 10:1280-1289.
19. M Zanetti, R Gennaro and D Romeo. Cathelicidins: a novel protein family with a common proregion and a variable C-terminal antimicrobial domain, FEBS Lett. (1995) 374:1-5.
20. Y Q Tang, J Yuan, G Osapay, K Osapay, D Tran, C J Miller, A J Ouellette, M E Selsted. A cyclic antimicrobial peptide produced in primate leukocytes by the ligation of two truncated alpha-defensins. Science (1999) 286:498-502.
21. X L Zhang, M E Selsted and A Pardi. NMR studies of defensin antimicrobial peptides. 1. Resonance assignment and secondary structure determination of rabbit NP-2 and human HNP-1. Biochemistry (1992) 31:11348-11356.
22. T Ganz, M E Selsted, D Szklarek, S S Harwig, K Daher, D F Bainton, R I Lehrer. Defensins. Natural peptide antibiotics of human neutrophils. J. Clin. Invest. (1985) 76:1427-1435.
23. C G Wilde, J E Griffith, M N Marra, J L Snable, R W Scott. Purification and characterization of human neutrophil peptide 4, a novel member of the defensin family. J. Biol. Chem. (1989) 264:11200-11203.
24. E B Mallow, A Harris, N Salzman, J P Russell, R J DeBardinis, E Ruchelli and C L Bevins. Human entric defensins. J. Biol. Chem. (1996) 271:4038-4045.
25. K W Bensch, M Raida, H J Mägert, P Schulz-Knappe, W G Forssmann. hBD-1: a novel beta-defensin from human plasma. FEBS Lett. (1995) 368:331-335.
26. W J Larrick, M Hirata, R F Balint, J Lee, J Zhong, S C Wright. Human CAP18: a novel antimicrobial lipopolysaccharide-binding protein. Infect. Immun. (1995) 63:1291-1297.
27. J B Cowland, A H Johnsen, N Borregaard. hCAP-18, a cathelin/pro-bactenecin-like protein of human neutrophil specific granules, FEBS Lett. (1995) 368:173-176.
28. B Agerberth, H Gunne, J Odeberg, P Kogner, H G Boman. FALL-39, a putative human peptide antibiotic, is cysteine-free and expressed in bone marrow and testis. Proc. Natl. Acad. Sci. USA (1995) 92:195-199.
29. G H Gudmundsson, B Agerberth, J Odebeg, T Bergman, B Olsson and R. Salcedo. The human gene FALL39 and processing of the cathelin precursor to the antibacterial peptide LL-37 in granulocytes. Eur. J. Biochem. (1996) 238:325-332.
30. R Gennaro, B Skerlavaj and D Romeo. Purification, composition, and activity of two bactenecins, antibacterial peptides of bovine neutrophils, Infect. Immun. (1989) 57:3142-3146.
31. B Agerberth, J Y Lee, T Bergman, M Carlquist, H G Boman, V Mutt, H Jörnvall. Amino acid sequence of PR-39. Eur. J. Biochem. (1991) 202:849-854.
32. R A Houghten. General method for the rapid solid-phase synthesis of large numbers of peptides: specificity of antigen-antibody interaction at the level of individual amino acids. Proc. Natl. Acad. Sci. USA (1985) 82:5131-5135
33. G Zhang, H Wu, J Shi, T Ganz, C R Ross, F Blecha. Molecular cloning and tissue expression of porcine β-defensin-1. FEBS Lett. (1998) 424:37-40.
34. G Zhang, H Hiraiwa, H Yasue, H Wu, C R Ross, D Troyer, F Blecha. Cloning and Characterization of the Gene for a New Epithelial β-Defensin. J. Biol. Chem. (1999) 274:24031-24037.
35. J Shi, G Zhang, H Wu, C R Ross, F Blecha, T Ganz. Porcine Epithelial β-Defensin 1 Is Expressed in the Dorsal Tongue at Antimicrobial Concentrations. Infect. Immun. (1999) 67:3121-3127
36. M D Edge, A R Greene, G R Heathcliffe, P A Meacock, W Schuch, D B Scanlon, T C Atkinson, C R Newton & A F Markham. Total synthesis of a human leukocyte interferon gene. Nature (1981) 292:756-762.
37. K P Nambiar, J Stackhouse, D M Stauffer, W P Kennedy, J K Eldredge, S A Benner. Total synthesis and cloning of a gene coding for the ribonuclease S protein. Science (1984) 223:1299-1301.
38. E Jay, D MacKnight, C Lutze-Wallace, D Harrison, P Wishart, W Y Liu, V Asundi, L Pomeroy-Cloney, J Rommens, L Eglington, et al. Chemical synthesis of a biologically active gene for human immune interferon-gamma. Prospect for site-specific mutagenesis and structure-function studies. J. Biol. Chem. (1984) 259:6311-6317.
39. S L Beaucage, M H Caruthers. Deoxynucleoside phosphoramidites—A new class of key intermediates for deoxypolynucleotide synthesis. Tet. Lett. (1981) 22:1859-1862.
40. M D Matteucci, M H Caruthers. Synthesis of deoxyoligonucleotides on a polymer support. J. Am. Chem. Soc. (1981) 103:3185-3191.
41. R Dijkema, P H van der Meide, P H Powels, M Caspers, M Dubbeld, H Schellekens. Cloning and expression of the chromosomal immune interferon gene of the rat (1985) EMBO J. 4:761
42. C M Gorman, G T Merlino, M C Willingham, I Pastan, B H Howard. The Rous sarcoma virus long terminal repeat is a strong promoter when introduced into a variety of eukaryotic cells by DNA-mediated transfection (1982) Proc. Natl. Acad. Sci. USA 79:6777
43. M Boshart, F Weber, G Jahn, K Dorsch-Hilsler, B Fleckenstein, W Schaffner. A very strong enhancer is located upstream of an immediate early gene of human cytomegalovirus (1985) Cell 41:521.
44. T A Kunkel. Rapid and efficient site-specific mutagenesis without phenotypic selection (1985) Proc. Natl. Acad. Sci. USA 82:448.
45. J Geisselsoder, F Witney, P Yuckenberg. Efficient site-directed in vitro mutagenesis (1987) BioTechniques 5:786.
46. M J Zoller and M Smith. Oligonucleotide-directed mutagenesis of DNA fragments cloned into M13 vectors. (1983)Methods Enzymol. 100:468.
47. Dalbadie-McFarland, L W Cohen, A D Riggs, C Morin, K Itakura, J H Richards. Oligonucleotide-directed mutagenesis as a general and powerful method for studies of protein function. (1982) Proc. Natl. Acad. Sci USA 79:6409.
48. A M Krieg, A-K Yi, S Matson, T J Waldschmidt, G A Bishop, R Teasdale, G A Koretzky & D M Klinman. Nature (1995) 374:546.
49. H L Davis, R Weeranta, T J Waldschmidt, L Tygrett, J Schorr, A M Krieg. CpG DNA Is a Potent Enhancer of Specific Immunity in Mice Immunized with Recombinant Hepatitis B Surface Antigen. J. Immunol. (1998) 160 (2) 870-876.
50. S P Kasturi, K Sachaphibulkij and K Roy, Biomaterials, Volume 26, Issue 32, 2005, pages 6375-6385.
51. R M Garavito, S. Ferguson-Miller, Detergents as Tools in Membrane Biochemistry. J Biol Chem. 276(35), 32403-32406, 2001.
52. A Medici, G Fantin, P Pedrini, M Gleria, and F Minto, Macromolecules, 1992, Vol. 25, No. 10, 2569.
53. H R Allcock, D C Ngo, M Parvez, K B Visscher, Inorg. Chem., 1994, 33 (10), pp 2090-2102.
54. C D Schmulbach, Prog. Inorg. Chem. 4, 275 1962.
55. R Dishon. JAm Chem Soc. 71, 2251 (1949).
56. R Rätz, H Schroeder, H Ulrich, E Kober and C Grundmann, J Amer. Chem Soc. 84 551 (1962).
57. G A Carriedo, L Fernández-Catuxo, F J Garcia Alonso, P Gómez Elipe, P A González and G Sánchez, J Appl Polymer Sci (1996) 59, 1879-1885.
58. Valsecchi R, Mutta F (WO2016037998)
59. B R Holzer, C Hatz, D Schmidt-Sissolak, R Glück, B Althaus and M Egger, Immunogenicity and adverse effects of inactivated virosome versus alum-adsorbed hepatitis A vaccine: a randomized controlled trial, Vaccine, Volume 14, Issue 10, 1996, Pages 982-986.
60. Y Poovorawan, A Theamboonlers, S Chumdermpadetsuk, R Glück and S J Cryz Jr. Safety, immunogenicity, and kinetics of the immune response to a single dose of virosome-formulated hepatitis A vaccine in Thais. Vaccine, Volume 13, Issue 10, 1995, Pages 891-893.
61. R A Shaw, B W Fitzsimmons, B C Smith, Chem. Rev. 62, 247 (1962).
62. Liebig-Wöhler, Briefwechsel vol. 1, 63; Ann. Chem. (Liebig), vol. 11 (1834), 14.
63. T S Desin, P K S Lam, B Koch, C Mickael, E Berberov, A L S Wisner, HGG Townsend, A A Potter, and W Köster. Infection and Immunity (2009). 2866-2875.

What is claimed is:

1. A compound of formula I: 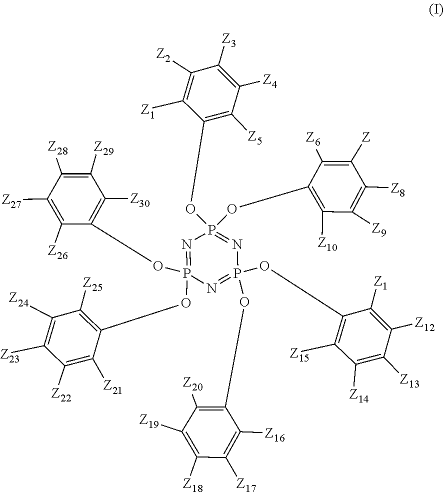
a tautomer, stereoisomer, polymorph, hydrate, solvate, or pharmaceutically acceptable salt thereof, wherein:
each of Z₁, Z₂, Z₃, Z₄, Z₅, Z₆, Z₇, Z₈, Z₉, Z₁₀, Z₁₁, Z₁₂, Z₁₃, Z₁₄, Z₁₅, Z₁₆, Z₁₇, Z₁₈, Z₁₉, Z₂₀, Z₂₁, Z₂₂, Z₂₃, Z₂₄, Z₂₅, Z₂₆, Z₂₇, Z₂₈, Z₂₉, and Z₃₀ are independently selected from H or formula (II): 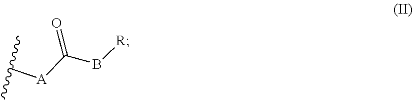
wherein at least one of Z₁₋₃₀ is represented by formula II, and each of Z₁₋₃₀ is identical or non-identical; and wherein:
A, if present, is selected from C₁-C₇ alkyl, C₂-C₇ alkenyl, C₂-C₇ alkynyl, O, S, and N,
wherein C₁-C₇ alkyl, C₂-C₇ alkenyl, and/or C₂-C₇ alkynyl are straight or branched and optionally substituted by one or more substituents selected from:
1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc),
B is selected from C₁-C₇ alkyl, C₂-C₇ alkenyl, C₂-C₇ alkynyl, H, O, S, and N,
wherein C₁-C₇ alkyl, C₂-C₇ alkenyl, and/or C₂-C₇ alkynyl are straight or branched and optionally substituted by one or more substituents selected from:
1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc),
R, if present, is selected from H, C₁-C₄₅ alkyl, C₂-C₄₅ alkenyl, and C₂-C₄₅ alkynyl,
wherein C₁-C₄₅ alkyl, C₂-C₄₅ alkenyl, and/or C₂-C₄₅ alkynyl are straight or branched and optionally substituted by one or more substituents selected from:
1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc).

2. The compound of claim 1, wherein:
A is C₂ alkyl; and
B is selected from O and N.

3. The compound of any one of claims 1-2, wherein R is selected from:
C₁-C₄₅ alkyl, C₁-C₄₅ alkenyl, and C₁-C₄₅ alkynyl,
wherein C₁-C₄₅ alkyl, C₂-C₄₅ alkenyl, and/or C₂-C₄₅ alkynyl are straight or branched and optionally substituted by one or more substituents selected from hydroxyl, 1° amino, 2° amino, 3° amino, 4° amino, carboxylic acid, amino fluorenylmethyloxycarbonyl (NH-Fmoc), tert-butyloxycarbonyl (Boc), and amino tert-butyloxycarbonyl (—NH—Boc).

4. The compound of any one of claims 1-3, wherein R is selected from:

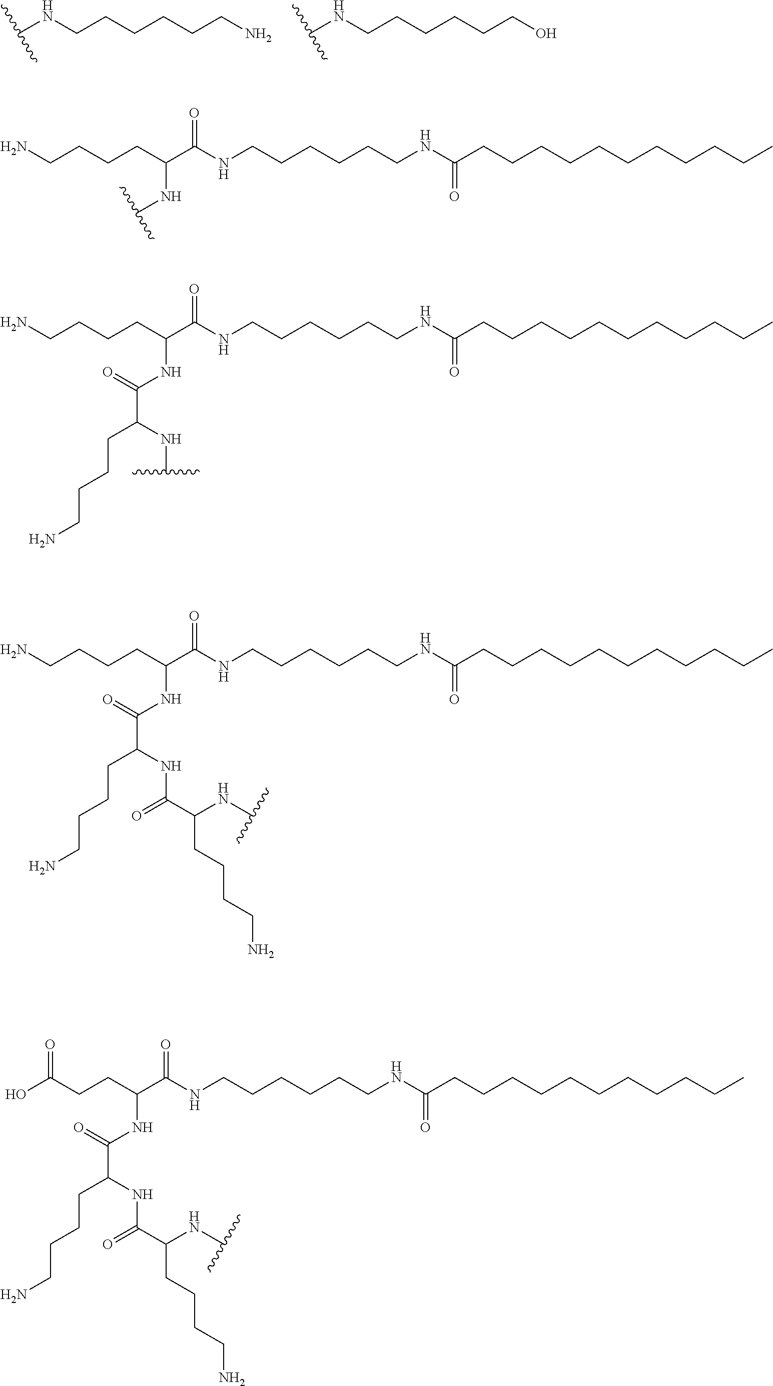 [SMILES: *NCCCCCCN ; *NCCCCCCO ; *NC(CCCCN)C(=O)NCCCCCCNC(=O)CCCCCCCCCCC ; *NC(CCCCN)C(=O)NC(CCCCN)C(=O)NCCCCCCNC(=O)CCCCCCCCCCC ; *NC(CCCCN)C(=O)NC(CCCCN)C(=O)NC(CCCCN)C(=O)NCCCCCCNC(=O)CCCCCCCCCCC ; *NC(CCCCN)C(=O)NC(CCCCN)C(=O)NC(CCC(=O)O)C(=O)NCCCCCCNC(=O)CCCCCCCCCCC]

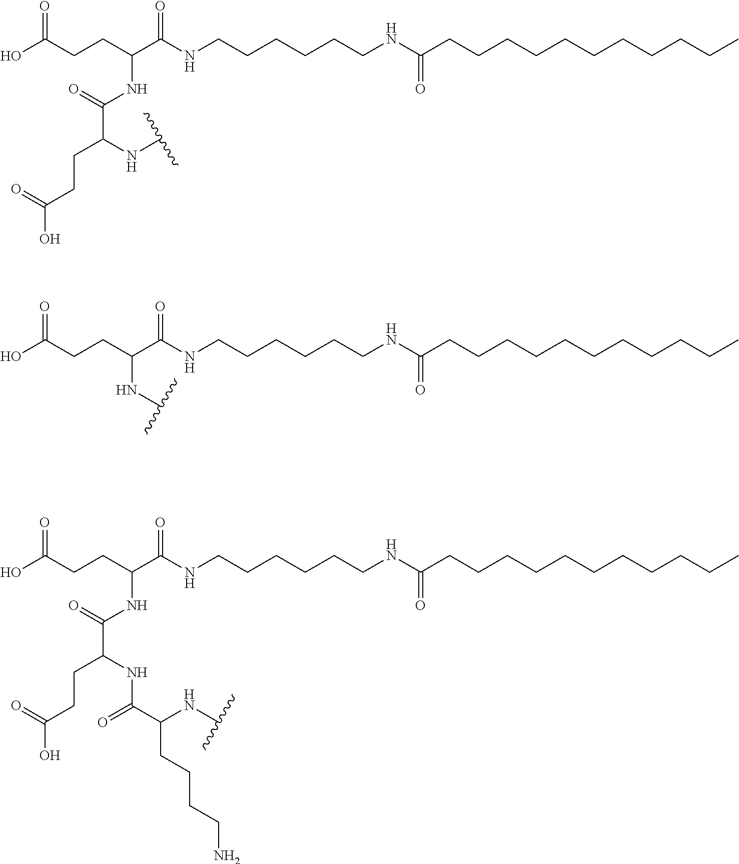 [SMILES: *NC(CCC(=O)O)C(=O)NC(CCC(=O)O)C(=O)NCCCCCCNC(=O)CCCCCCCCCCC ; *NC(CCC(=O)O)C(=O)NCCCCCCNC(=O)CCCCCCCCCCC ; *NC(CCCCN)C(=O)NC(CCC(=O)O)C(=O)NC(CCC(=O)O)C(=O)NCCCCCCNC(=O)CCCCCCCCCCC]

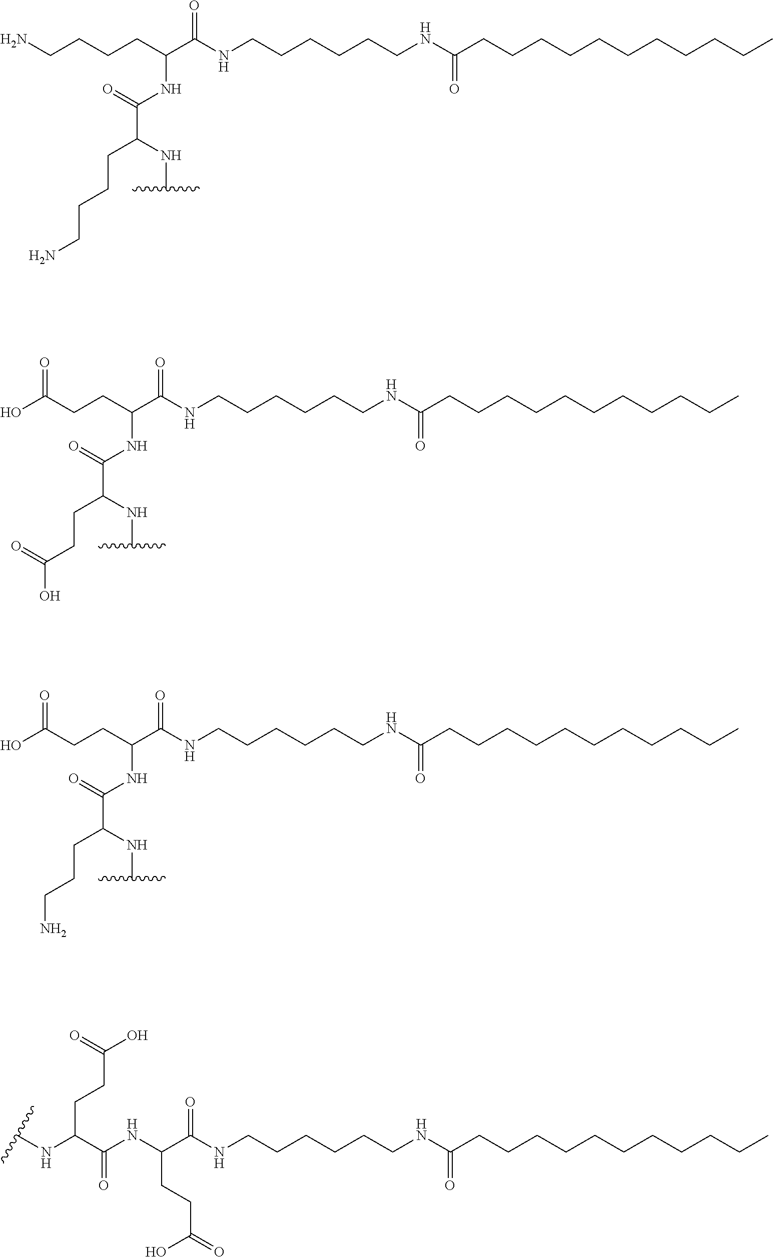 [SMILES: *NC(CCCCN)C(=O)NC(CCCCN)C(=O)NCCCCCCNC(=O)CCCCCCCCCCC ; *NC(CCC(=O)O)C(=O)NC(CCC(=O)O)C(=O)NCCCCCCNC(=O)CCCCCCCCCCC ; *NC(CCCN)C(=O)NC(CCC(=O)O)C(=O)NCCCCCCNC(=O)CCCCCCCCCCC ; *NC(CCC(=O)O)C(=O)NC(CCC(=O)O)C(=O)NCCCCCCNC(=O)CCCCCCCCCCC]

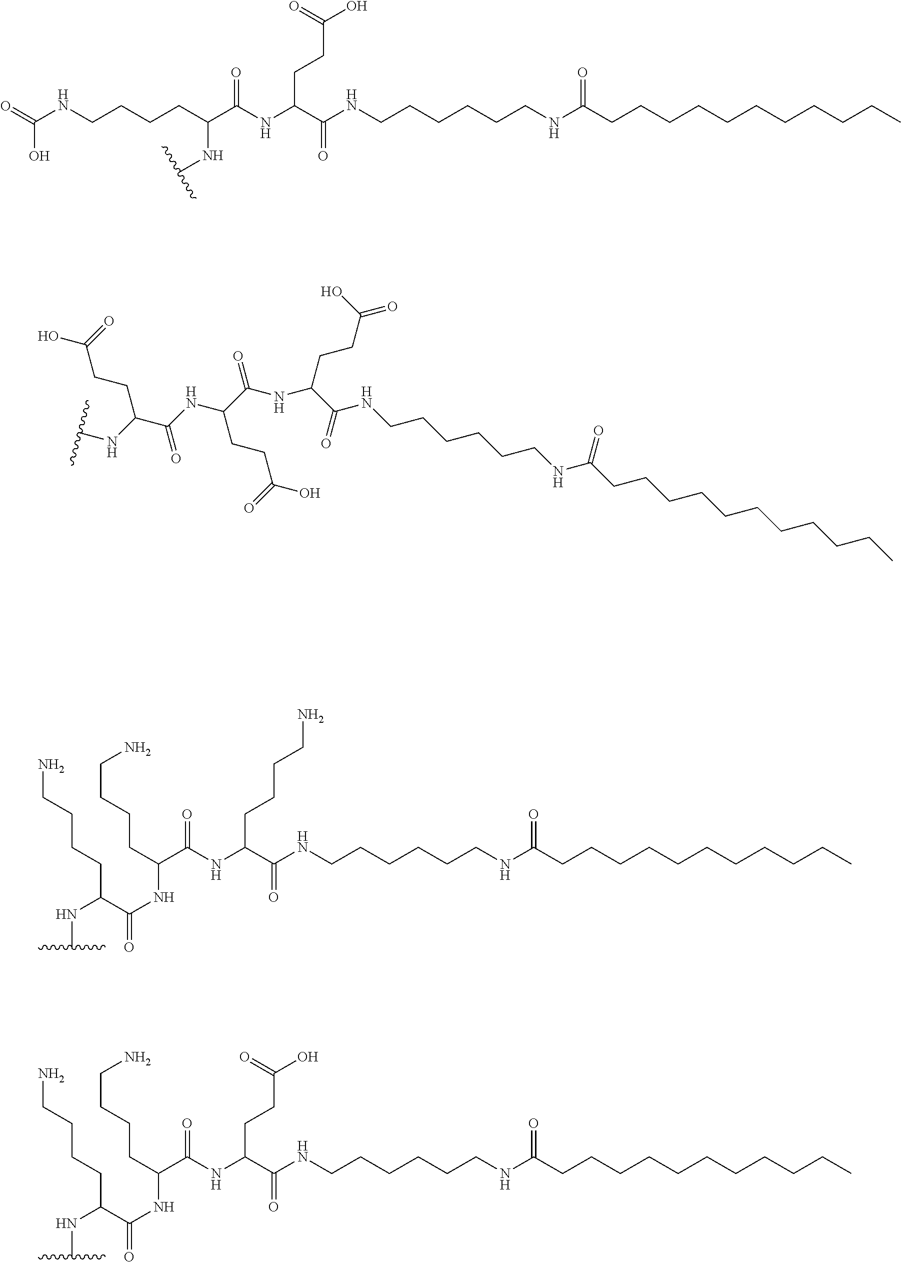 [SMILES: *NC(CCCCNC(=O)O)C(=O)NC(CCC(=O)O)C(=O)NCCCCCCNC(=O)CCCCCCCCCCC ; *NC(CCC(=O)O)C(=O)NC(CCC(=O)O)C(=O)NC(CCC(=O)O)C(=O)NCCCCCCNC(=O)CCCCCCCCCCC ; *NC(CCCCN)C(=O)NC(CCCCN)C(=O)NC(CCCCN)C(=O)NCCCCCCNC(=O)CCCCCCCCCCC ; *NC(CCCCN)C(=O)NC(CCCCN)C(=O)NC(CCC(=O)O)C(=O)NCCCCCCNC(=O)CCCCCCCCCCC]

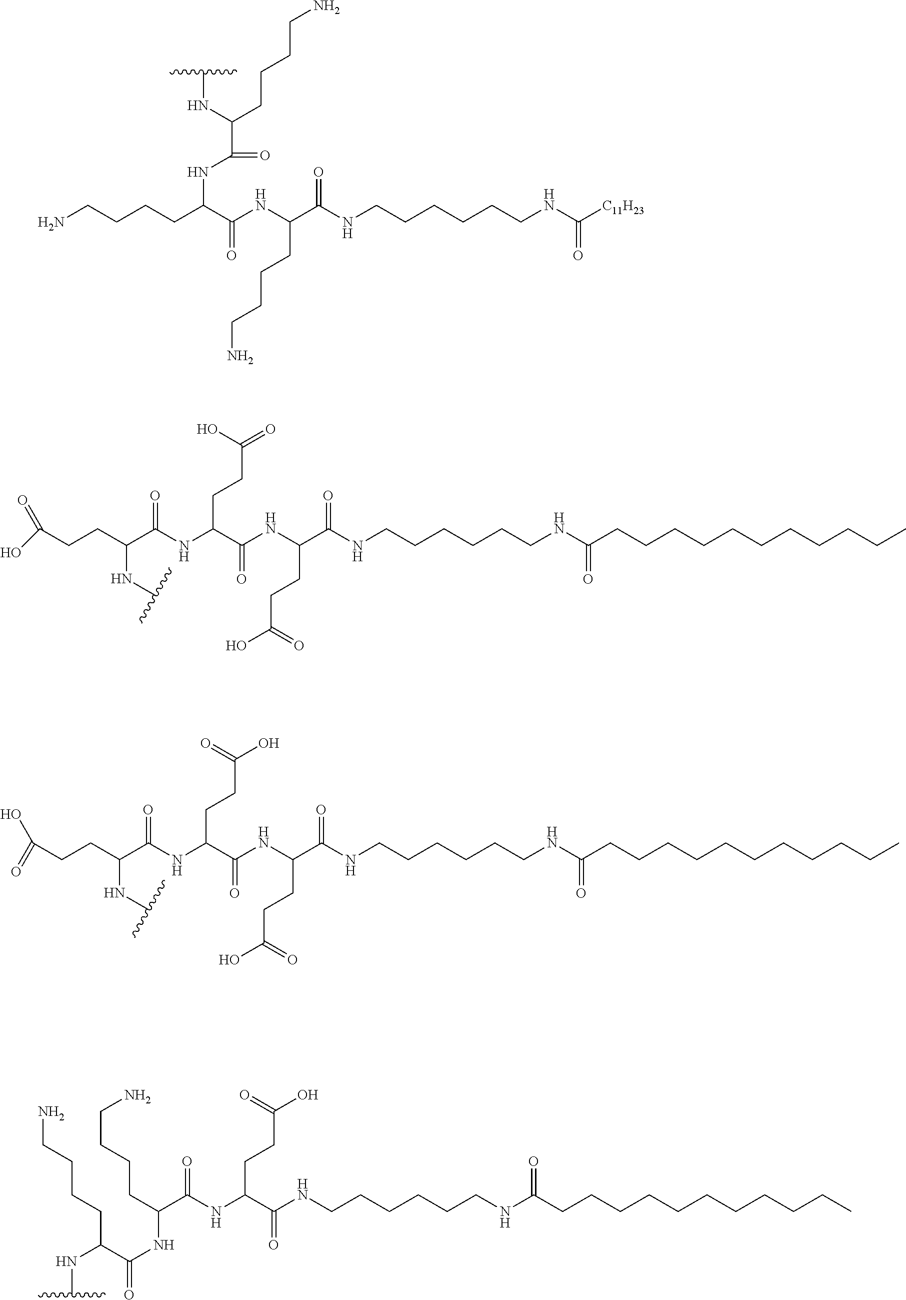

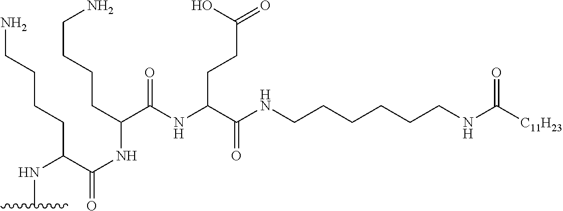

5. The compound of any one of claims 1-4, wherein:
A is C₂ alkyl;
B is N; and
R is:

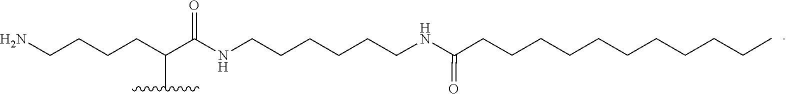 [SMILES: *C(CCCCN)C(=O)NCCCCCCNC(=O)CCCCCCCCCCC]

6. A compound having formula 37: 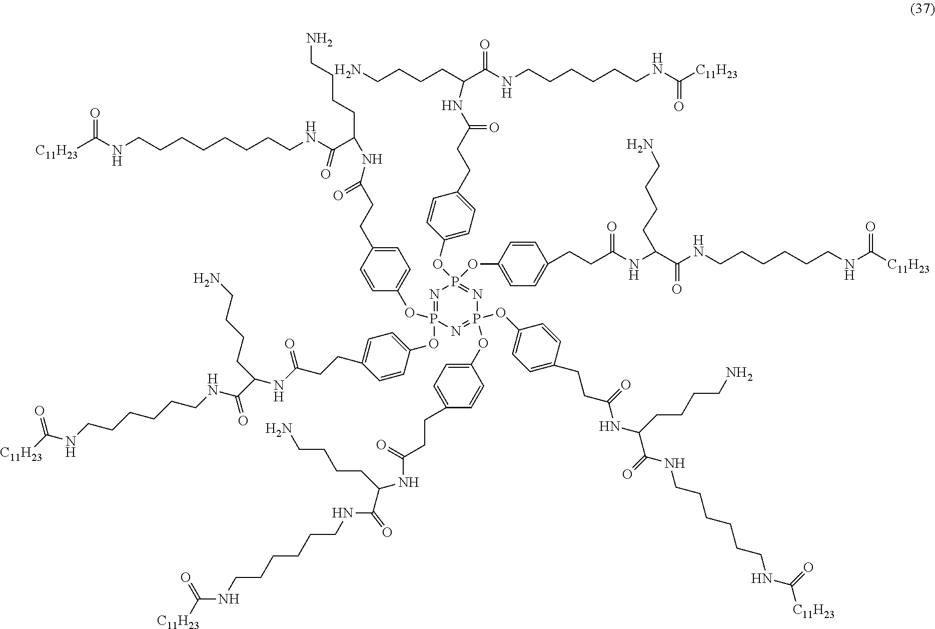
a tautomer, stereoisomer, polymorph, hydrate, solvate, or pharmaceutically acceptable salt thereof.

7. A compound having formula 6: 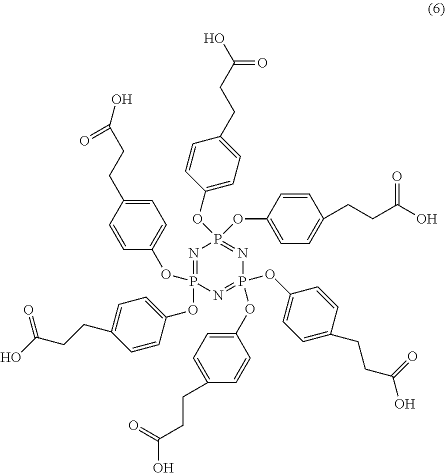 [SMILES: O=C(O)CCc1ccc(OP2(Oc3ccc(CCC(=O)O)cc3)=NP(Oc3ccc(CCC(=O)O)cc3)(Oc3ccc(CCC(=O)O)cc3)=NP(Oc3ccc(CCC(=O)O)cc3)(Oc3ccc(CCC(=O)O)cc3)=N2)cc1]
a tautomer, stereoisomer, polymorph, hydrate, solvate, or pharmaceutically acceptable salt thereof.

8. A compound of formula 11: 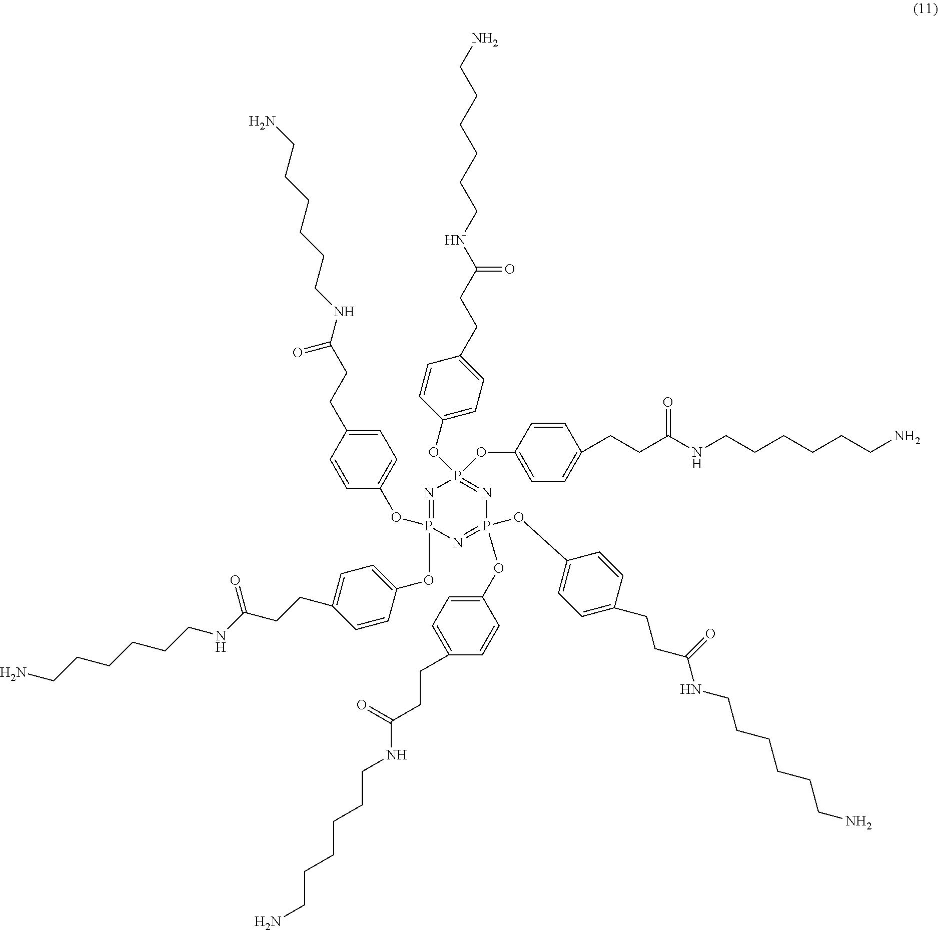 [SMILES: NCCCCCCNC(=O)CCc1ccc(OP2(Oc3ccc(CCC(=O)NCCCCCCN)cc3)=NP(Oc3ccc(CCC(=O)NCCCCCCN)cc3)(Oc3ccc(CCC(=O)NCCCCCCN)cc3)=NP(Oc3ccc(CCC(=O)NCCCCCCN)cc3)(Oc3ccc(CCC(=O)NCCCCCCN)cc3)=N2)cc1]
a tautomer, stereoisomer, polymorph, hydrate, solvate, or pharmaceutically acceptable salt thereof.

9. A compound of formula 9: 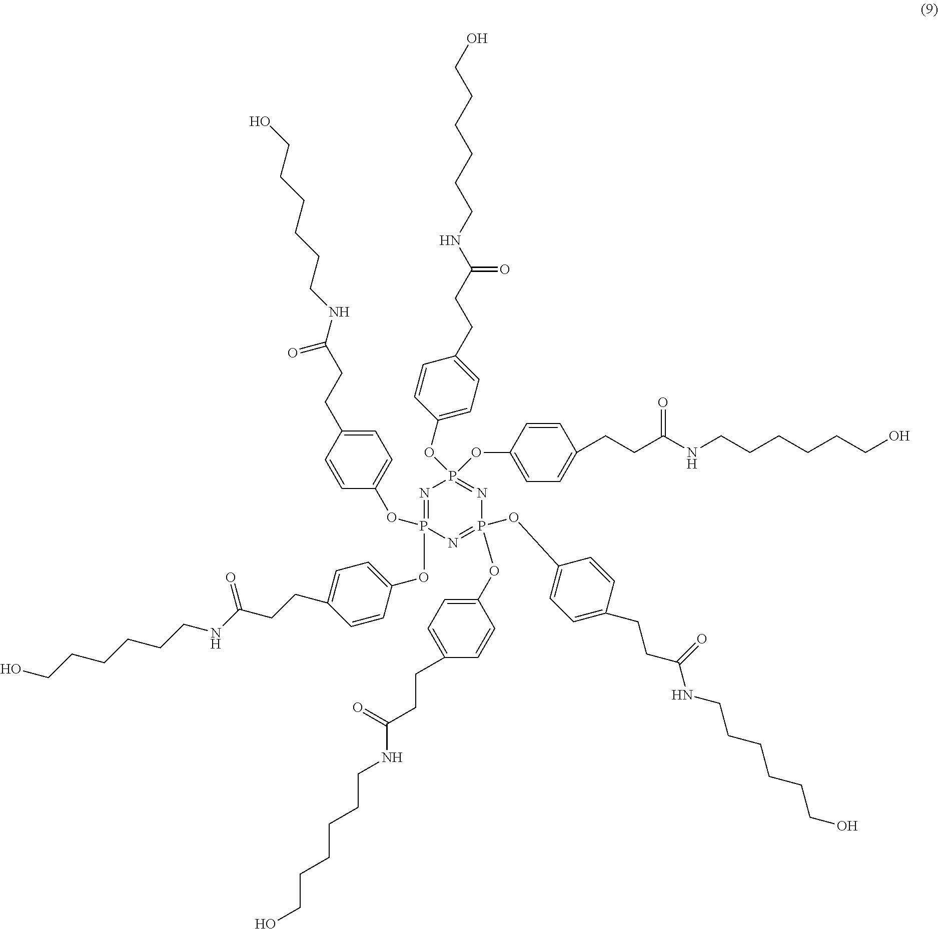 [SMILES: O=C(CCc1ccc(OP2(Oc3ccc(CCC(=O)NCCCCCCO)cc3)=NP(Oc3ccc(CCC(=O)NCCCCCCO)cc3)(Oc3ccc(CCC(=O)NCCCCCCO)cc3)=NP(Oc3ccc(CCC(=O)NCCCCCCO)cc3)(Oc3ccc(CCC(=O)NCCCCCCO)cc3)=N2)cc1)NCCCCCCO]
a tautomer, stereoisomer, polymorph, hydrate, solvate, or pharmaceutically acceptable salt thereof.

10. A compound of formula 39: 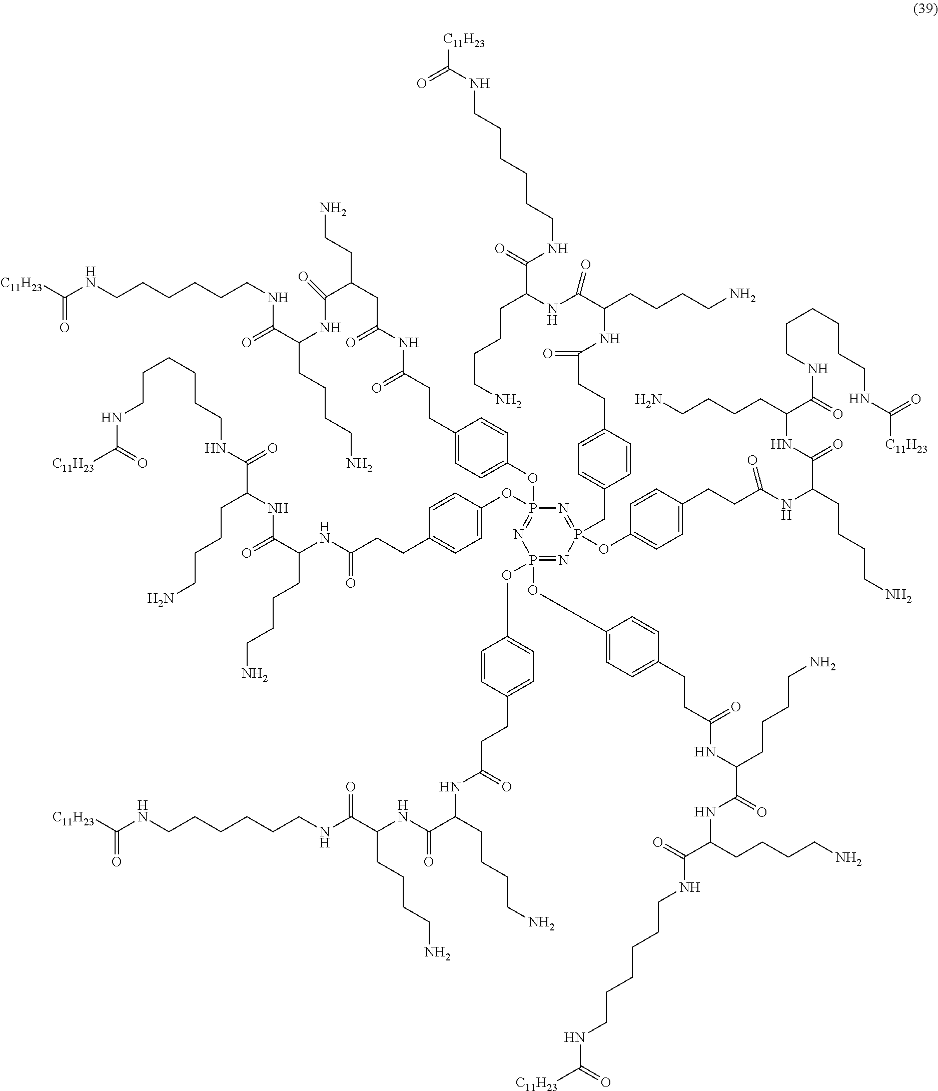
a tautomer, stereoisomer, a polymorph, hydrate, solvate, or pharmaceutically acceptable salt thereof.

11. Aa oligomeric structure comprising two or more compounds as defined in any one of claims 1-10.

12. The oligomeric structure of claim 11, wherein the two or more compounds are linked via an amide or ester bond.

13. A method for preparing a compound as defined in any one of claims 1-10, said method comprising:
providing a reactant of formula VI: 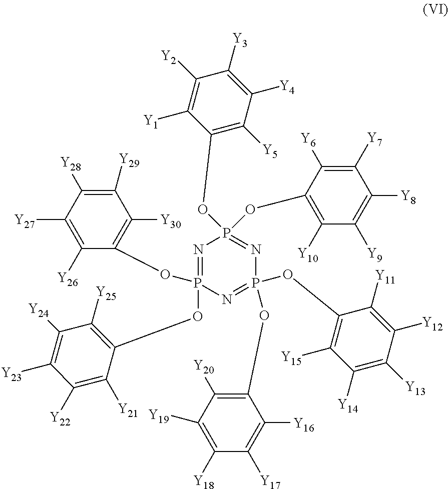
wherein each of Y₁₋₃₀ is independently selected from H and formula (III): 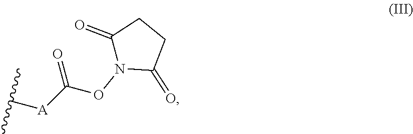
wherein at least one of Y₁₋₃₀ is represented by formula III and each of Y₁₋₃₀ is identical or non-identical;
performing a substitution reaction with a C₂-C₄₅ nucleophile; wherein
A, if present, is selected from C₁-C₇ alkyl, C₂-C₇ alkenyl, C₂-C₇ alkynyl, O, S, and N,
wherein C₁-C₇ alkyl, C₂-C₇ alkenyl, and/or C₂-C₇ alkynyl are straight or branched and optionally substituted by one or more substituents selected from:
1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc),
the C₂-C₄₅ nucleophile is linear or branched C₂-C₄₅ alkyl, C₂-C₄₅ alkenyl, and/or C₂-C₄₅ alkynyl and optionally substituted by one or more substituents selected from:
1° amino, 2° amino, 3° amino, 4° amino, acetal, acyl halide, acyl, aldehyde, alkoxyl, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc).

14. The method of claim 13, wherein the reactant comprises formula 7: 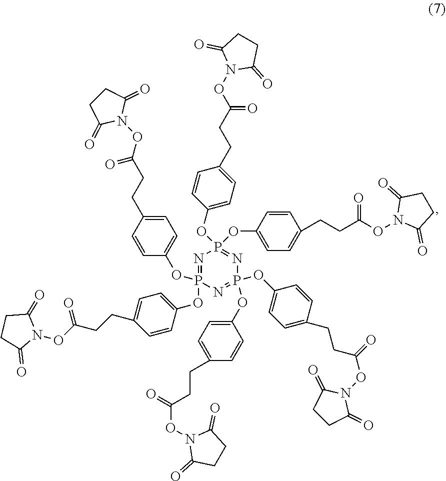 [SMILES: O=C(CCc1ccc(OP2(Oc3ccc(CCC(=O)ON4C(=O)CCC4=O)cc3)=NP(Oc3ccc(CCC(=O)ON4C(=O)CCC4=O)cc3)(Oc3ccc(CCC(=O)ON4C(=O)CCC4=O)cc3)=NP(Oc3ccc(CCC(=O)ON4C(=O)CCC4=O)cc3)(Oc3ccc(CCC(=O)ON4C(=O)CCC4=O)cc3)=N2)cc1)ON1C(=O)CCC1=O]
and the C₂-C₄₅ nucleophile is a C₂-C₄₅ amine,
wherein the C₂-C₄₅ amine is linear or branched C₂-C₄₅ alkyl, C₂-C₄₅ alkenyl, and/or C₂-C₄₅ alkynyl and optionally substituted by one or more substituents selected from:
1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc).

15. The method of any one of claims 13-14, wherein the C₂-C₄₅ nucleophile is one or more of:

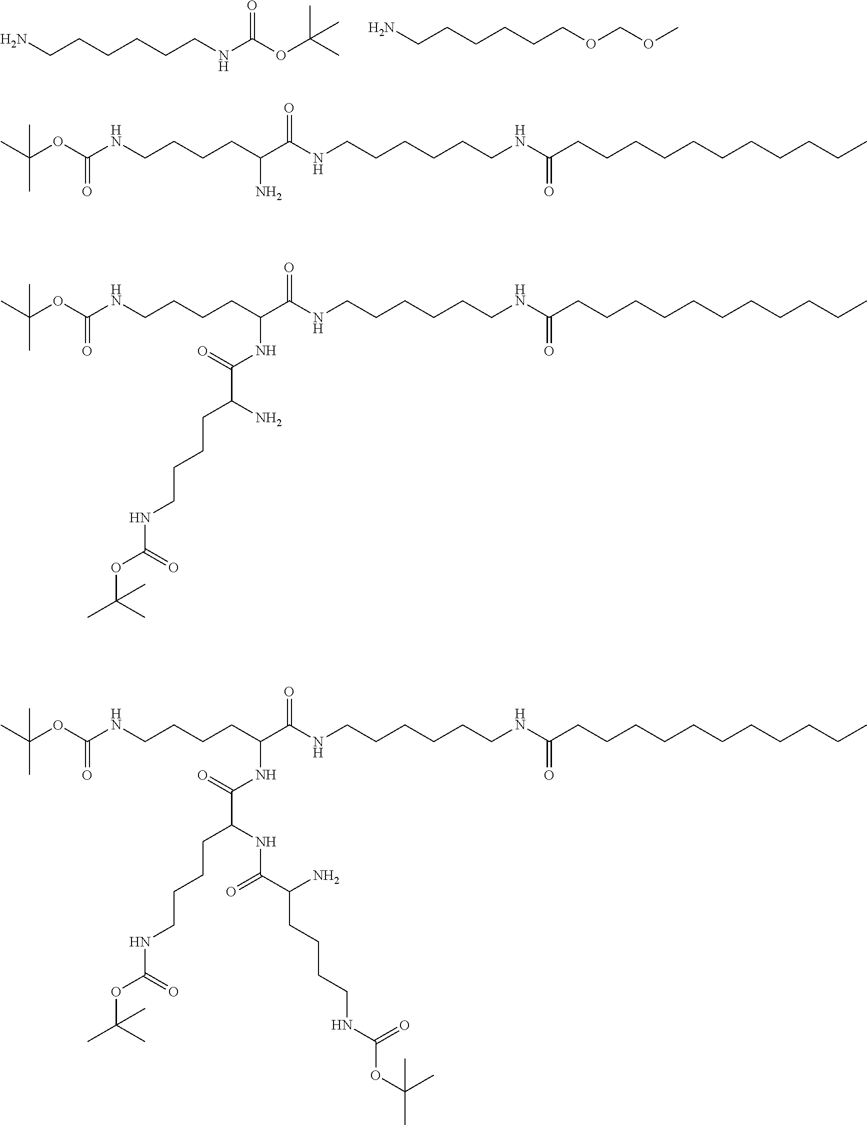

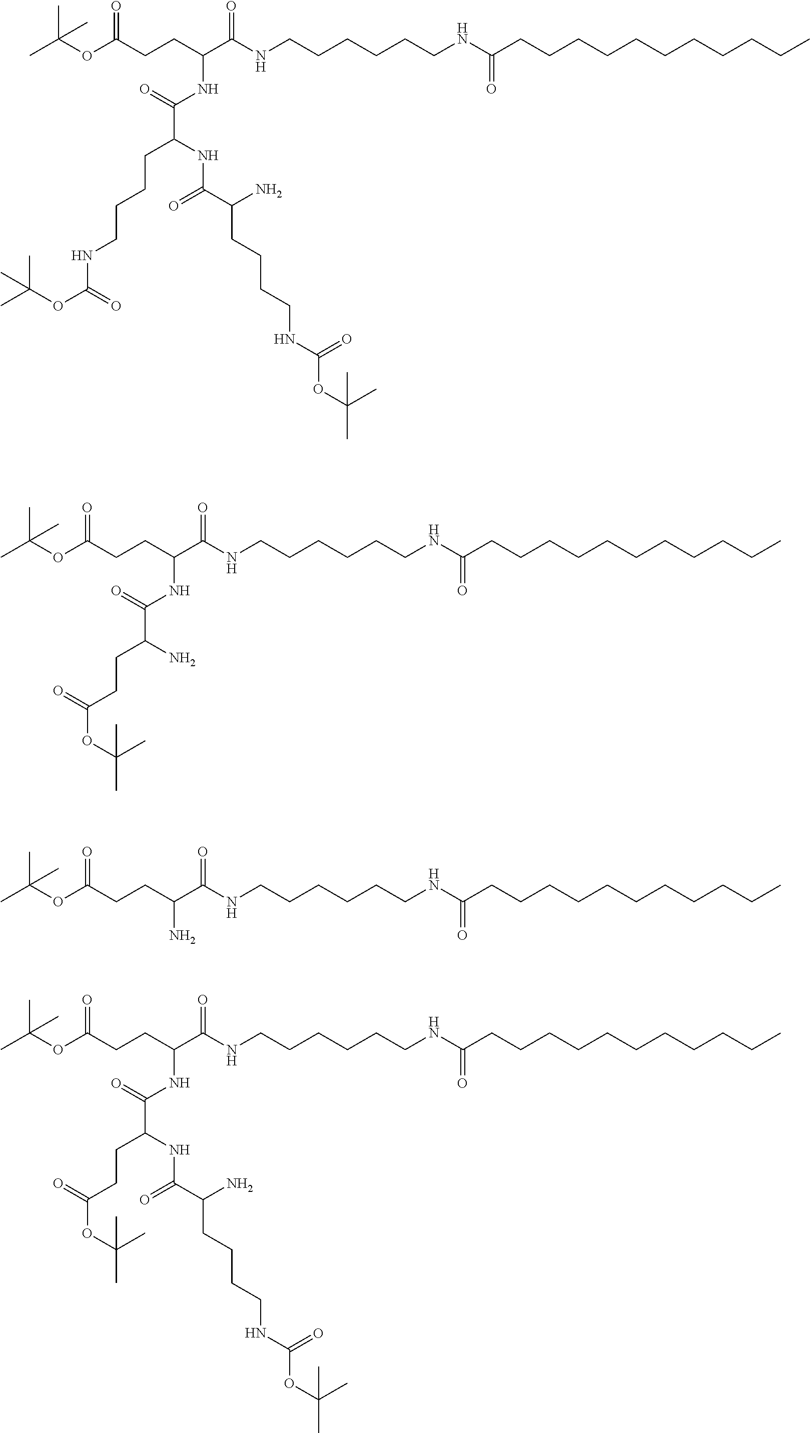

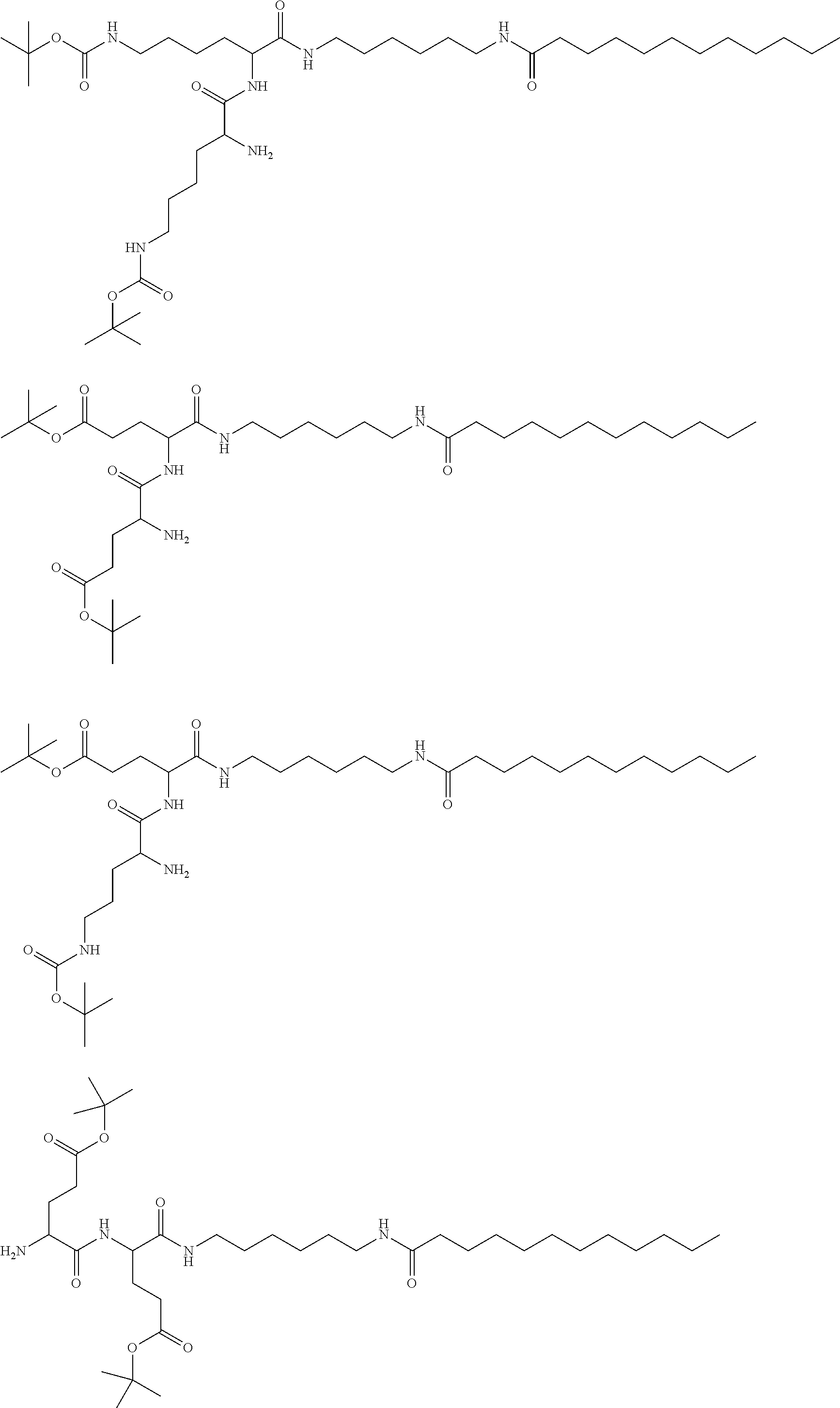

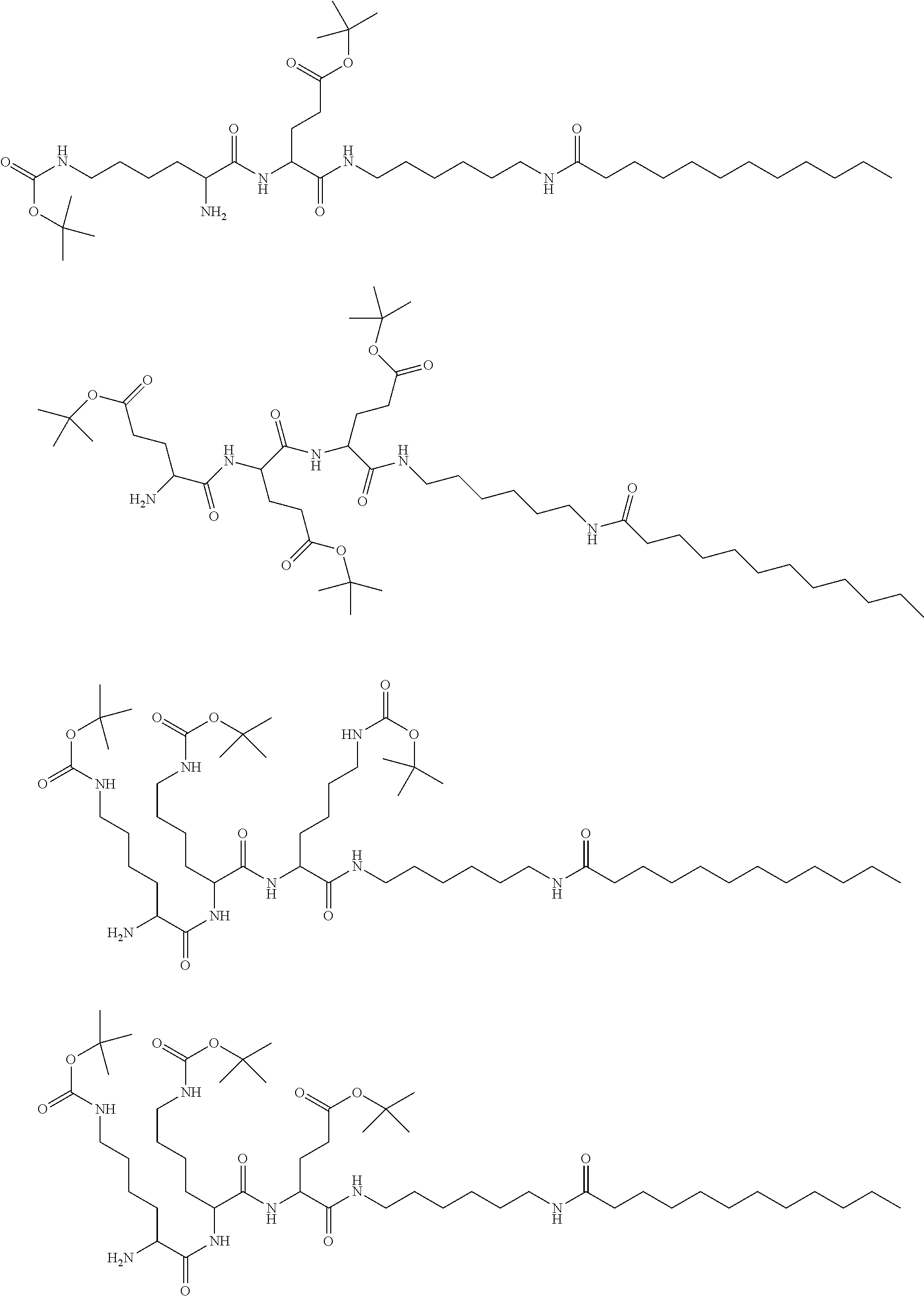

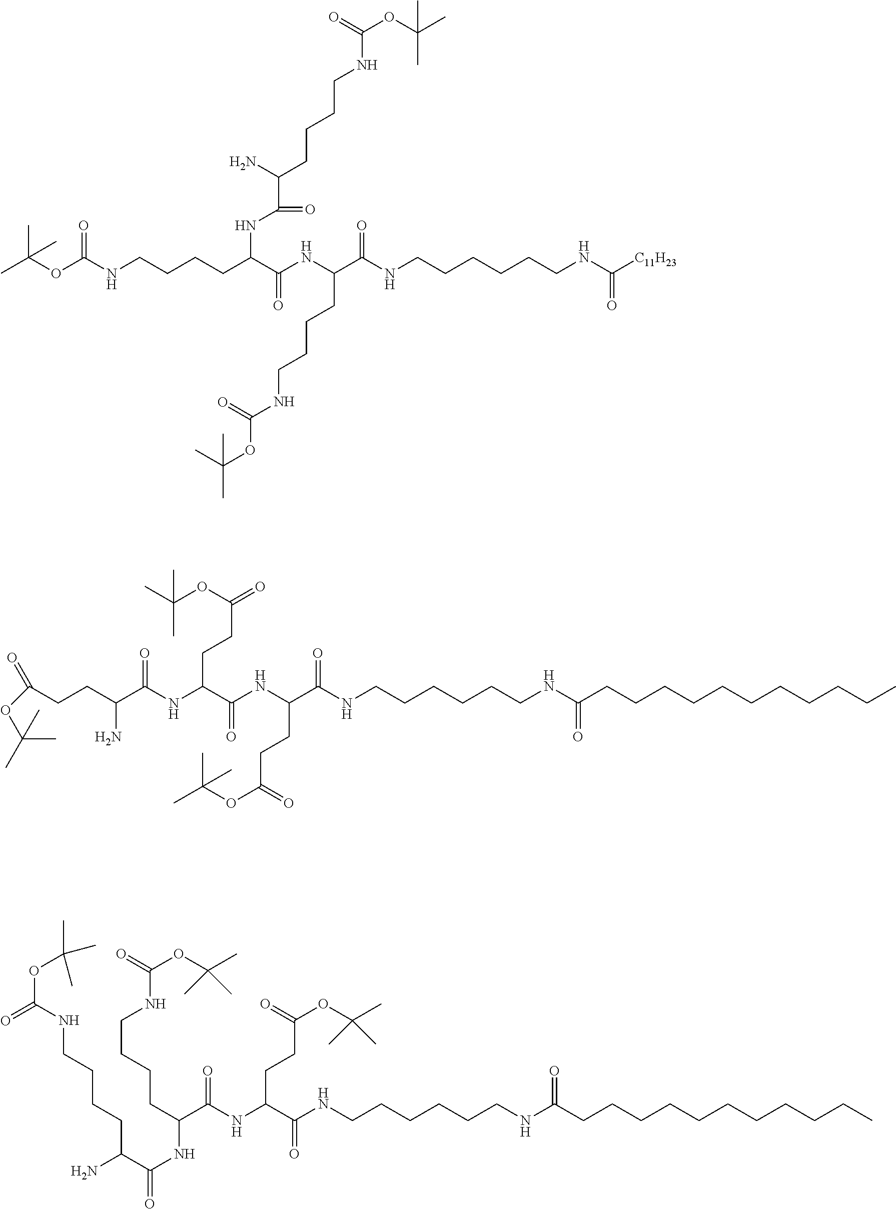

16. The method of any one of claims 13-15, wherein the method further comprises a deprotection step comprising an aqueous acid at a pH of 1 or less.

17. A method of producing a compound of any one of claims 1-10, the method comprising:
providing a first reactant of formula 3: 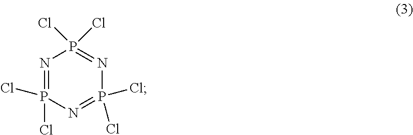 [SMILES: ClP1(Cl)=NP(Cl)(Cl)=NP(Cl)(Cl)=N1]
performing a first substitution reaction with formula IX: 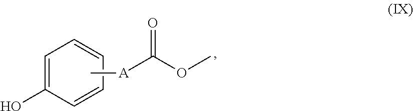
the first reactant, and a base to yield a first intermediate;
performing a hydrolysis reaction with a hydroxide salt and the first intermediate to yield a second intermediate;
performing a second substitution reaction with the second intermediate, N-hydroxysuccinimide and N,N′-Diisopropylcarbodiimide (DIPCDI) to yield a third intermediate; and
performing a third substitution reaction with a C₂-C₄₅ nucleophile and the third intermediate, wherein:
A is selected from C₁-C₇ alkyl, C₂-C₇ alkenyl, C₂-C₇ alkynyl,
wherein C₁-C₇ alkyl, C₂-C₇ alkenyl, and/or C₂-C₇ alkynyl are straight or branched and optionally substituted by one or more substituents selected from:
1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc),
the C₂-C₄₅ nucleophile is a linear or branched C₂-C₄₅ alkyl, C₂-C₄₅ alkenyl, and/or C₂-C₄₅ alkynyl and optionally substituted by one or more substituents selected from:
1° amino, 2° amino, 3° amino, 4° amino, acetal, acyl halide, acyl, aldehyde, alkoxy, amide, aryl, azide, carbamimidoyl, carboxylic acid, cyano, disulfide, epoxide, ester, ether, hydroxyl, imide, imine, ketone, nitrile, nitro, oxime, peroxide, sulfonic acid, sulphonamidyl, thioester, thioether, thiol, amino fluorenylmethyloxycarbonyl (NH-Fmoc), tert-butyloxycarbonyl (Boc), and amino tert-butyloxycarbonyl (—NH—Boc).

18. The method of claim 17, wherein the first substitution reaction further comprises nBu₄, N⁺Br⁻ (TBAB) and the base is K₂CO₃.

19. The method of any one of claims 17-18, wherein the hydroxide salt is sodium hydroxide.

20. The method of any one of claims 17-19, wherein the first intermediate comprises formula X:

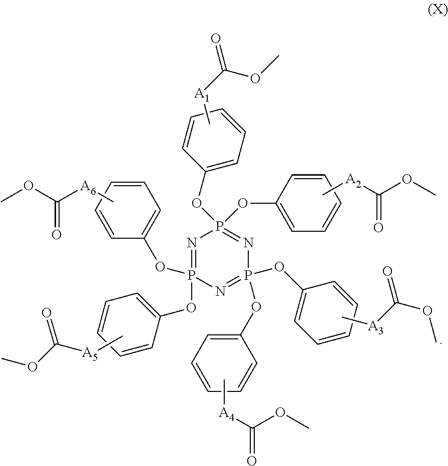

21. The method of any one of claims 17-20, wherein the second intermediate comprises formula XI:

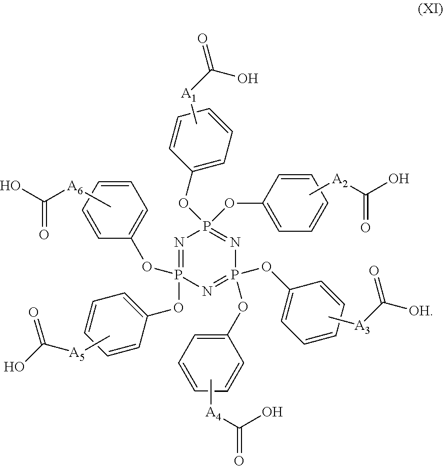

22. The method of any one of claims 17-21, wherein the third intermediate comprises formula VI:

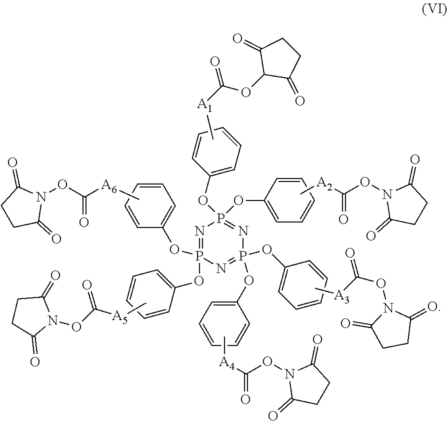

23. The method of any one of claims 17-22, wherein the C₂-C₄₅ nucleophile is one or more of:

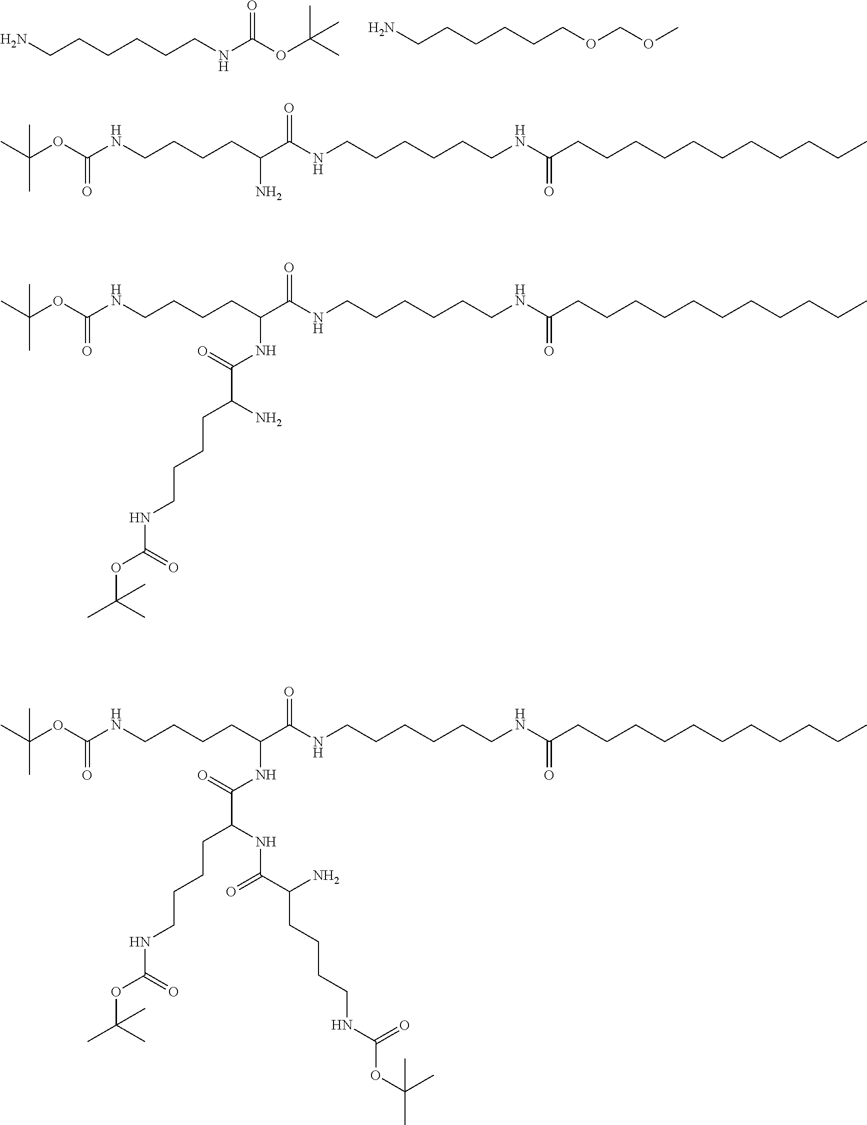

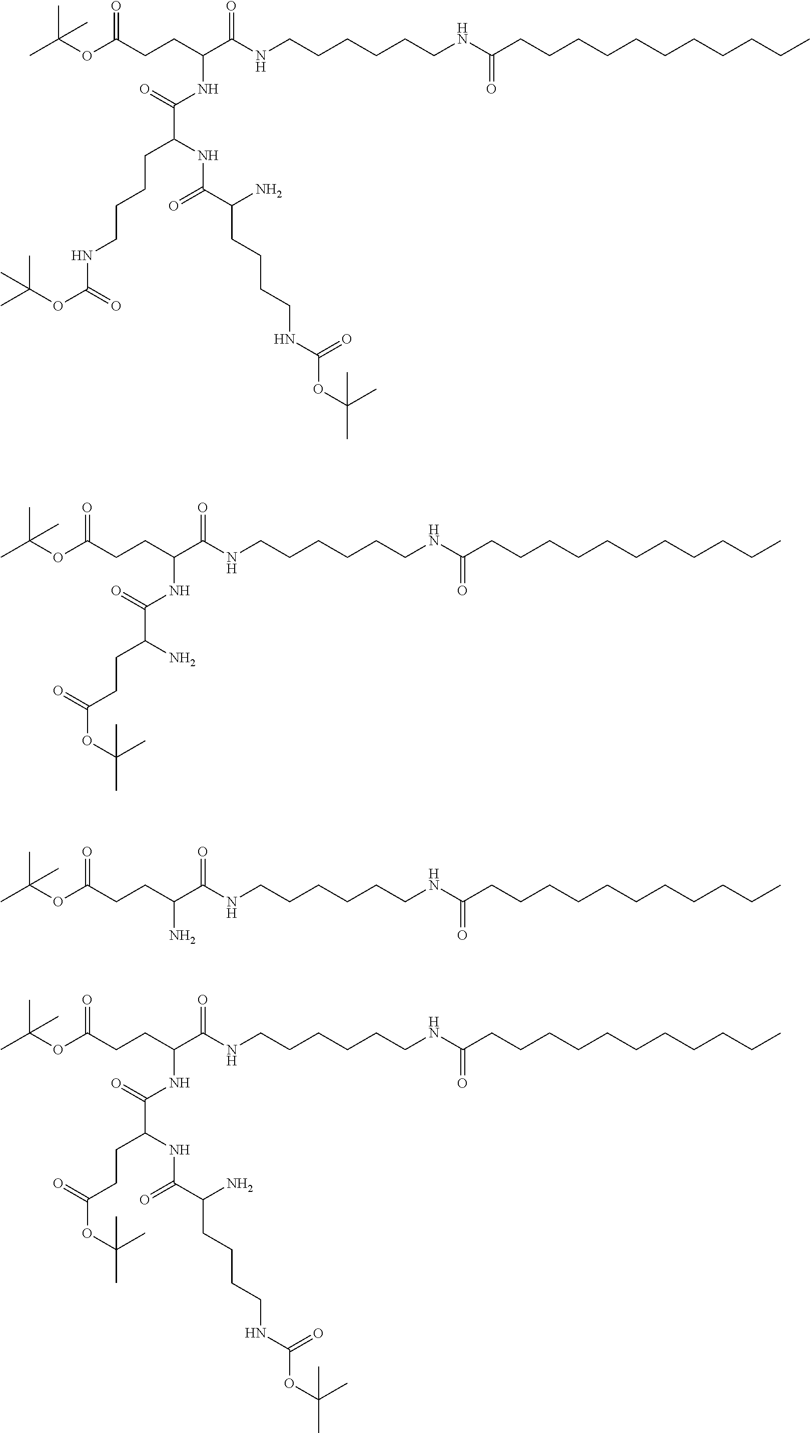

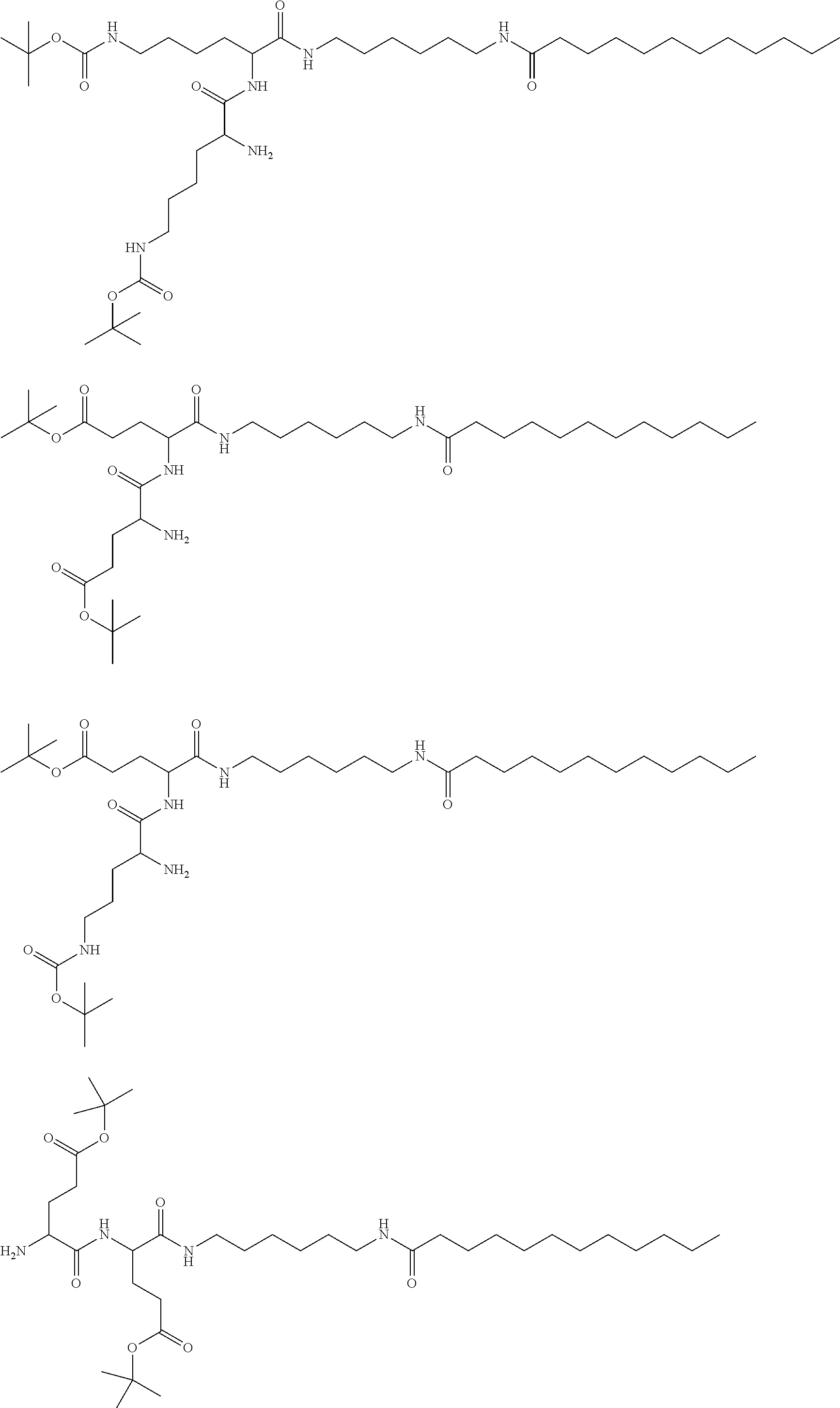

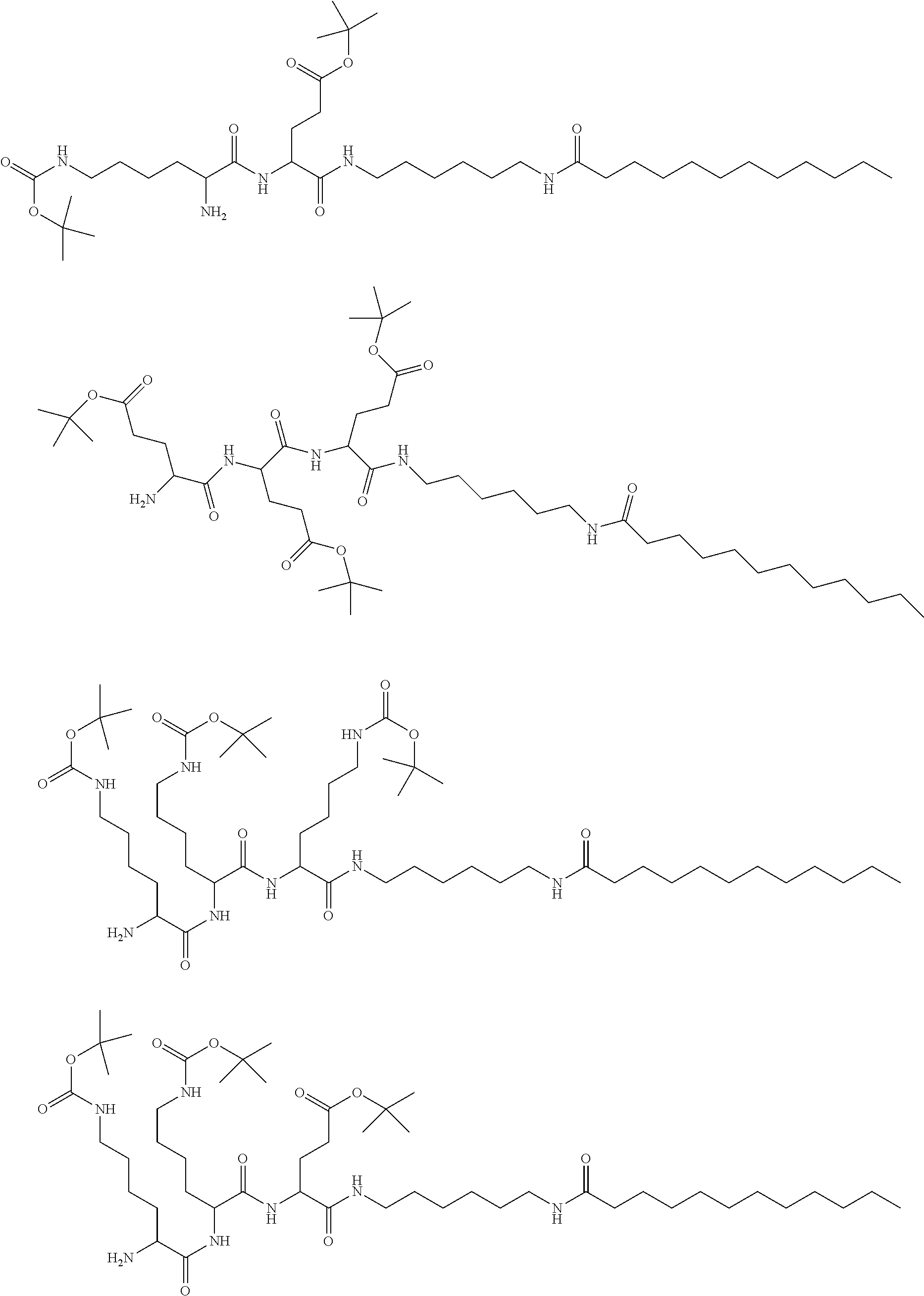

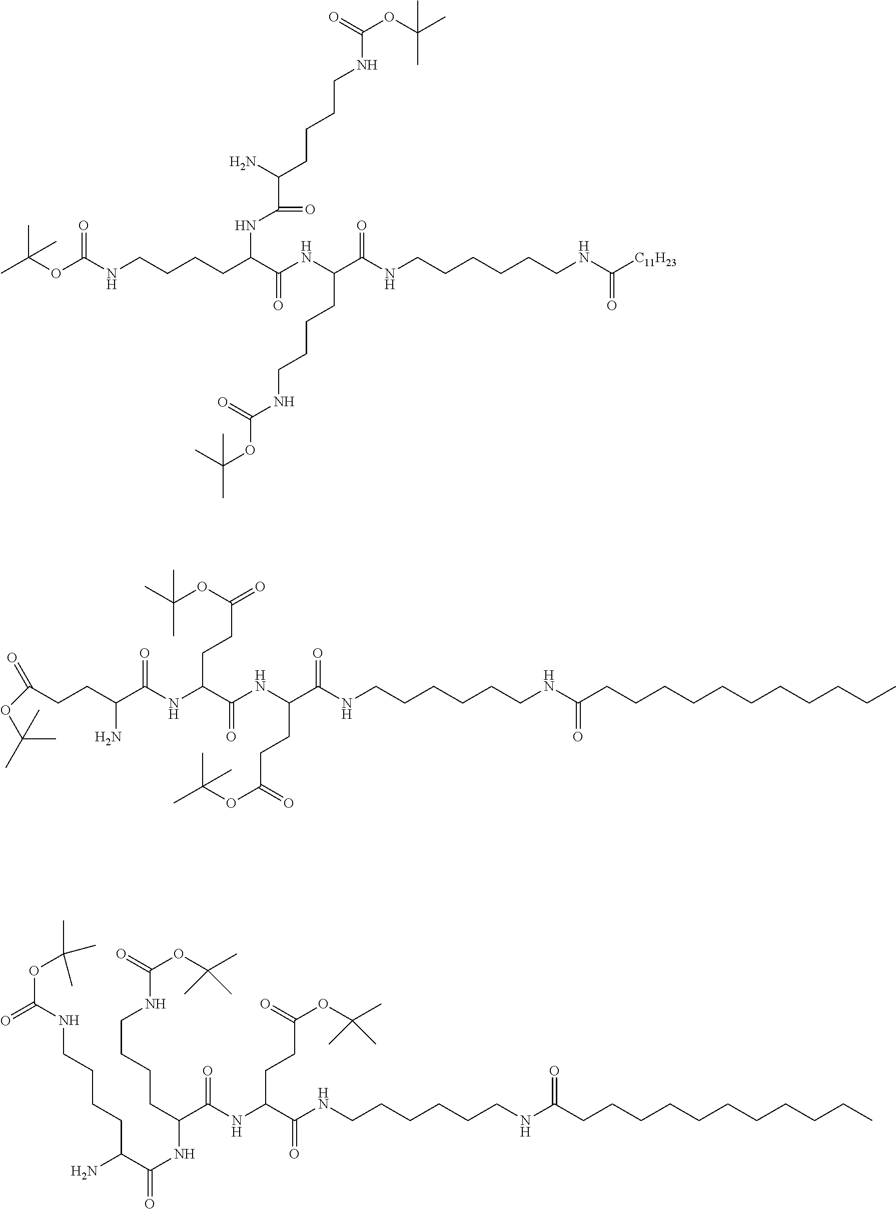

24. The method of any one of claims 17-23, wherein the method further comprises acidic work-up at a pH of 1 or less.

25. A method of producing a compound of formula X: 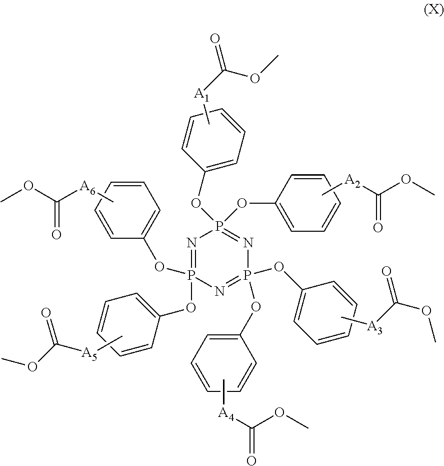
the method comprising:
providing a reactant of formula VIII: 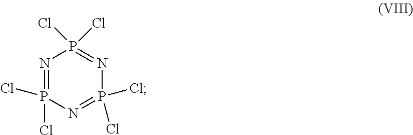 [SMILES: ClP1(Cl)=NP(Cl)(Cl)=NP(Cl)(Cl)=N1]
performing a substitution reaction with formula IX: 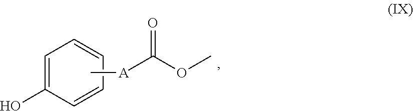
to the reactant, and a base.

26. The method of claim 25, wherein the substitution reaction further comprises nBu₄, N⁺Br⁻ (TBAB).

27. The method of any one of claims 25-26, wherein the base is K₂CO₃.

28. A method of producing a compound of formula XI: 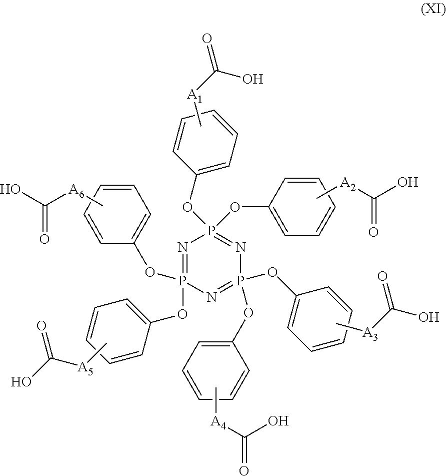
the method comprising:
providing a reactant of formula X: 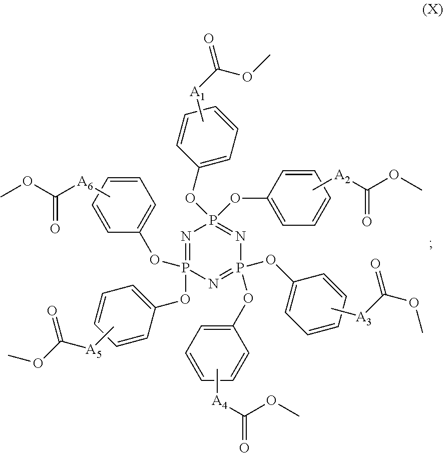
performing a hydrolysis reaction with a hydroxide salt and the reactant.

29. The method of claim 28, wherein the hydroxide salt is sodium hydroxide.

30. A method of producing a compound of formula VI: 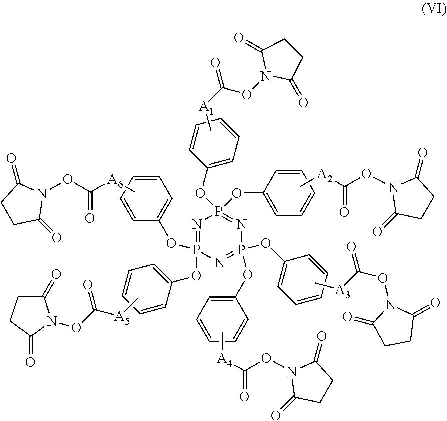
the method comprising:
providing a reactant of formula XI: 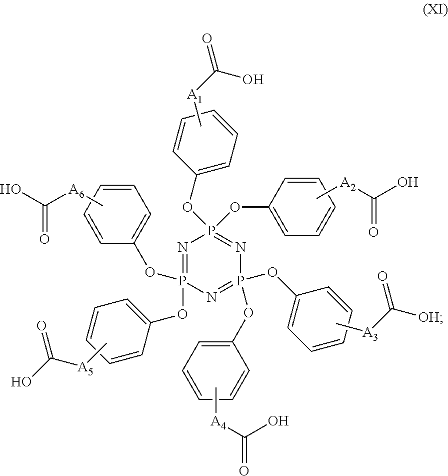
performing a substitution reaction with N-hydroxysuccinimide, N,N′-Diisopropylcarbodiimide (DIPCDI) and the reactant.

31. A method of producing a compound of formula 5: 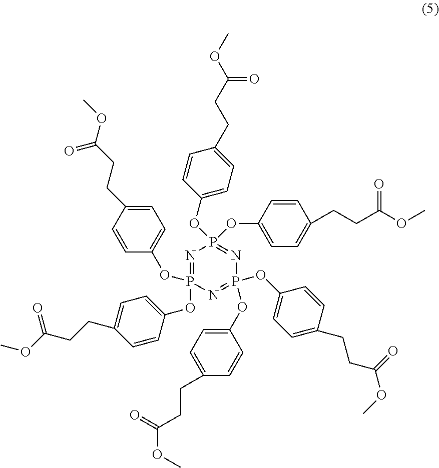
the method comprising:
providing a reactant of formula 3: 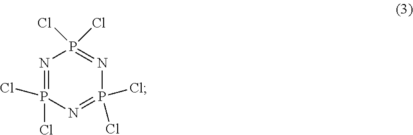 [SMILES: ClP1(Cl)=NP(Cl)(Cl)=NP(Cl)(Cl)=N1]
performing a substitution reaction with formula 4: 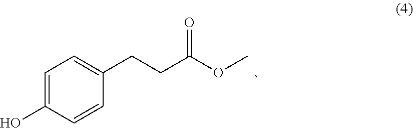
the reactant and a base.

32. The method of claim 31, wherein the substitution reaction further comprises nBu₄, N⁺Br⁻ (TBAB).

33. The method of any one of claims 31-32, wherein the base is K₂CO₃.

34. A method of producing a compound of formula 6: 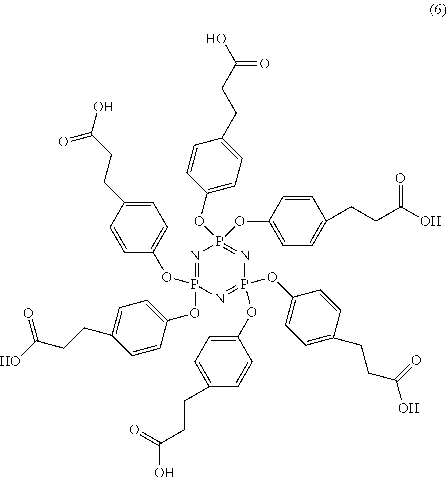 [SMILES: O=C(O)CCc1ccc(OP2(Oc3ccc(CCC(=O)O)cc3)=NP(Oc3ccc(CCC(=O)O)cc3)(Oc3ccc(CCC(=O)O)cc3)=NP(Oc3ccc(CCC(=O)O)cc3)(Oc3ccc(CCC(=O)O)cc3)=N2)cc1]
the method comprising:
providing a reactant of formula 5: 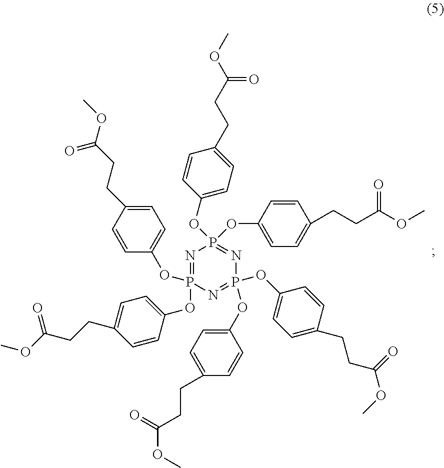
and
performing a hydrolysis reaction with sodium hydroxide and the reactant.

35. A method of producing a compound of formula 7: 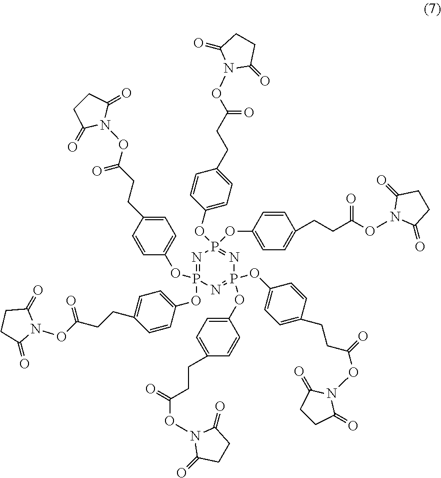 [SMILES: O=C(CCc1ccc(OP2(Oc3ccc(CCC(=O)ON4C(=O)CCC4=O)cc3)=NP(Oc3ccc(CCC(=O)ON4C(=O)CCC4=O)cc3)(Oc3ccc(CCC(=O)ON4C(=O)CCC4=O)cc3)=NP(Oc3ccc(CCC(=O)ON4C(=O)CCC4=O)cc3)(Oc3ccc(CCC(=O)ON4C(=O)CCC4=O)cc3)=N2)cc1)ON1C(=O)CCC1=O]
the method comprising:
providing a reactant of formula 6: 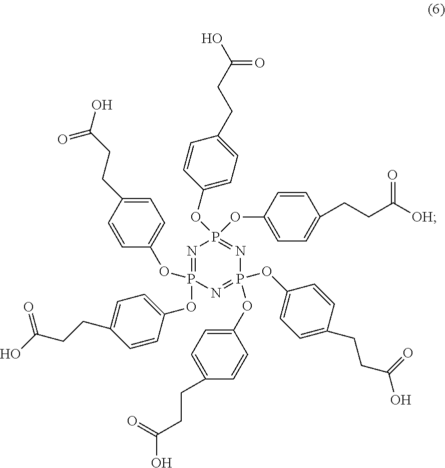 [SMILES: O=C(O)CCc1ccc(OP2(Oc3ccc(CCC(=O)O)cc3)=NP(Oc3ccc(CCC(=O)O)cc3)(Oc3ccc(CCC(=O)O)cc3)=NP(Oc3ccc(CCC(=O)O)cc3)(Oc3ccc(CCC(=O)O)cc3)=N2)cc1]
performing a substitution reaction with N-hydroxysuccinimide, N,N′-Diisopropylcarbodiimide (DIPCDI) and the reactant.

36. A method of producing a compound of formula 37: 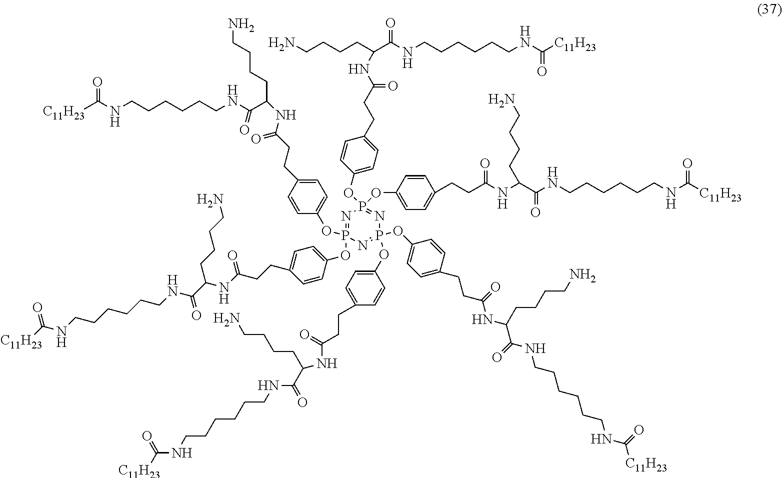
the method comprising:
providing a first reactant of formula 7: 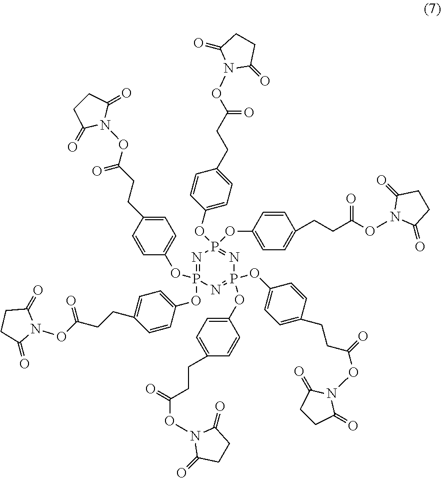 [SMILES: O=C(CCc1ccc(OP2(Oc3ccc(CCC(=O)ON4C(=O)CCC4=O)cc3)=NP(Oc3ccc(CCC(=O)ON4C(=O)CCC4=O)cc3)(Oc3ccc(CCC(=O)ON4C(=O)CCC4=O)cc3)=NP(Oc3ccc(CCC(=O)ON4C(=O)CCC4=O)cc3)(Oc3ccc(CCC(=O)ON4C(=O)CCC4=O)cc3)=N2)cc1)ON1C(=O)CCC1=O]
performing a substitution reaction with a second reactant of formula 19: 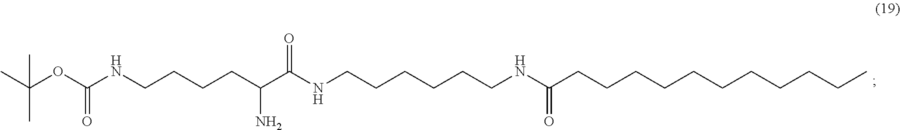 [SMILES: CCCCCCCCCCCC(=O)NCCCCCCNC(=O)C(N)CCCCNC(=O)OC(C)(C)C]
and
removing the t-butyl carbamate group with acid.

37. Use of a compound as defined in any one of claims 1-10 as an immunomodulator.

38. An adjuvant composition comprising:
a compound as defined in any one of claims 1-10, and a pharmaceutically acceptable excipient, carrier or diluent.

39. The adjuvant composition of claim 38, further comprising a host defense peptide.

40. The adjuvant composition of claim 39, wherein the host defense peptide is IDR-1002 (SEQ ID NO:19).

41. The adjuvant composition of any one of claims 38-40, further comprising an immunostimulatory sequence.

42. The adjuvant composition of claim 41, wherein the immunostimulatory sequence is poly(I:C).

43. The adjuvant composition of any one of claims 38-42, wherein the compound is the compound of formula 37 as defined in claim 6.

44. The adjuvant composition of any one of claims 38-42, wherein the compound is the compound of formula 39 as defined in claim 10.

45. The adjuvant composition of any one of claims 38-44, further comprising an antigen.

46. The adjuvant composition of claim 45, wherein the antigen is from a virus, bacteria, parasite, prion or fungus.

47. An adjuvant composition comprising:
a host defense peptide, an immunostimulatory sequence and a compound as defined in any one of claims 1-10.

48. An adjuvant composition comprising:
a host defense peptide and a compound as defined in any one of claims 1-10.

49. An adjuvant composition comprising:
an immunostimulatory sequence and a compound as defined in any one of claims 1-10.

50. The adjuvant composition of any one of claims 47-49, wherein the compound is the compound of formula 37 as defined in claim 6.

51. The adjuvant composition of any one of claims 47-49, wherein the compound is the compound of formula 39 as defined in claim 10.

52. The adjuvant composition of any one of claims 47-51, wherein the host defense peptide is IDR-1002 (SEQ ID NO:19).

53. The adjuvant composition of any one of claims 47-52, wherein the immunostimulatory sequence is poly(I:C).

54. The adjuvant composition of any one of claims 47-53, further comprising an antigen.

55. The adjuvant composition of claim 54, wherein the antigen is from a virus, bacteria, parasite, prion or fungus.

56. A composition comprising:
the adjuvant composition of any one of claims 47-55 and a pharmaceutically acceptable excipient, diluent, or carrier.

57. A method of enhancing an immune response to a selected antigen, said method comprising administering to a subject the composition of any one of claims 38-48 or claim 54.

58. The method of claim 57, wherein said antigen is formulated with said composition for administration to said subject.

59. The method of any one of claims 57-58, wherein administration comprises systemic and mucosal administration.

60. The method of claim 59, wherein systemic administration comprises intramuscular administration.

61. The method of claim 59, wherein systemic administration comprises oral administration.

62. The method of claim 59, wherein mucosal administration comprises intranasal, respiratory, buccal and genital.
